(19) United States
(12) Patent Application Publication
(10) Pub. No.: US 2022/0280561 A1
(43) Pub. Date: Sep. 8, 2022

(54) COPPER-ATSM FOR TREATING NEURODEGENERATIVE DISORDERS ASSOCITED WITH MITOCHONDRIAL DYSFUNCTION

(71) Applicant: RESEARCH INSTITUTE AT NATIONWIDE CHILDREN'S HOSPITAL, Columbus, OH (US)

(72) Inventors: Kathrin Christine Meyer, Columbus, OH (US); Cassandra Nicole Dennys-Rivers, Westerville, OH (US)

(21) Appl. No.: 17/637,986

(22) PCT Filed: Aug. 31, 2020
(86) PCT No.: PCT/US20/48779

§ 371 Date: Feb. 24, 2022

Related U.S. Application Data
(60) Provisional application No. 63/062,945, filed on Aug. 7, 2020.
(60) Provisional application No. 62/943,131, filed on Dec. 3, 2019.
(60) Provisional application No. 62/894,622, filed on Aug. 30, 2019.

(51) Int. Cl. A61K 33/34; A61K 31/555

(52) U.S. Cl. CPC A61K 33/34; A61K 31/555

(57) ABSTRACT

Provided herein are methods of treating a subject comprising a SCN2A mutation, a mutated SCN2A voltage-gated sodium channel protein, a SLC6A1 mutation, a SCN1A mutation or an IRF2BPL mutation. Methods of treating a subject with elevated levels of basal mitochondrial respiration, mitochondrial ATP-linked respiration, or a combination thereof, are also provided. Methods of treating a subject with a seizure disorder and method of treating a subject with a neurodegenerative or neurological disorder with mitochondrial dysfunction, optionally, with elevated levels of basal and/or ATP-linked respiration, are provided. Methods of improving survival of motor neurons or other neuronal cells types, reducing mitochondrial basal and/or ATP-linked respiration, reducing cellular oxidative stress, or a combination thereof, additionally are provided. In exemplary embodiments, the method comprises administering to the subject copper-ATSM (CuATSM) in an effective amount.

### CROSS REFERENCE TO RELATED APPLICATIONS AND INCORPORATION BY REFERENCE OF MATERIAL SUBMITTED ELECTRONICALLY

This application hereby claims priority to U.S. Provisional Patent Application No. 62/894,622, filed on Aug. 30, 2019; U.S. Provisional Patent Application No. 62/943,131, filed on Dec. 3, 2019; and U.S. Provisional Patent Application No. 63/062,945, filed on Aug. 7, 2020, which are incorporated by reference in their entirety.

Incorporated by reference in its entirety is a computer-readable nucleotide/amino acid sequence listing submitted concurrently herewith and identified as follows: 58,564 byte ASCII (Text) file named “54510_Seqlisting.txt”; created on Aug. 28, 2020.

### BACKGROUND

Amyotrophic lateral sclerosis (ALS) is a deadly adult onset neurodegenerative disease. Only 10% of ALS cases are linked to genetic mutations and are inherited (familial ALS) whereas the other 90% of patients develop the disease due to an unknown cause (sporadic ALS). Thus, the heterogeneous nature of ALS is not effectively represented in a single transgenic mouse model, and utilizing such model systems risks developing a targeted therapy for a small, mutation specific, patient subpopulation. Most clinical trials do not account for these patient subpopulations in trial design which makes interpretation of patient outcomes extremely difficult. This is one reason why many effective preclinical treatments for ALS fail in clinical trial.

To date, only Riluzole and Edaravone have received FDA approval for the treatment of ALS. However, Riluzole is only capable of modestly extending lifespan, whereas Edaravone only has significant impact on patient quality of life. CuATSM has been shown to have a profound impact on survival of the mutant SOD1 mouse model and its safe use in humans has also demonstrated. However, the impact CuATSM has on other types of ALS and other diseases, e.g., neurodegenerative diseases, has yet to be assessed.

### SUMMARY

Provided herein for the first time are data demonstrating that a specific patient ALS subpopulation responded favorably to copper-ATSM (CuATSM) therapy. All responders had a common pathway dysregulated, and, importantly, this pathway was corrected upon treatment with CuATSM. In the study described herein, patient skin biopsies were used to grow primary skin fibroblasts. These cells were then reprogrammed using a direct conversion method (Meyer et al., PNAS 829-832 (2014)) to produce induced neuronal progenitor cells (iNPCs). The iNPCs were differentiated into induced astrocytes (iAstrocytes) which were co-cultured with GFP⁺ motor neurons. This system was used to evaluate the therapeutic potential of CuATSM on both sporadic and familial ALS iAstrocytes. This co-culture system allowed for prediction of which patient subpopulations respond effectively to CuATSM, suggesting a promising future use in the field of personalized medicine for ALS. Thus, the disclosure provides for methods of identifying subjects who will respond to CuATSM therapy. As the pathway is common to other disorders of the nervous system, it is proposed, without being bound to any particular theory, that CuATSM therapy is used for the treatment of other disorders of the nervous system in which the pathway is dysregulated. One example is patients carrying mutations in the SCN2A gene. By studying patient skin cell-derived iAstrocytes with SCN2A mutations, it was found that they have the same mitochondrial dysfunction observed in ALS patients responding to CuATSM therapy.

Accordingly, the present disclosure provides methods of treating a subject comprising a SCN2A mutation, a mutated SCN2A voltage-gated sodium channel protein, a SLC6A1 mutation, a SCN1A mutation or a mutation in IRF2BPL. In exemplary embodiments, the method comprises administering to the subject CuATSM in an amount effective to treat the subject.

Also provided herein are methods of treating a subject with mitochondrial changes (e.g., changes in mitochondrial function, relative to a control, e.g., elevated levels of basal mitochondrial respiration, elevated mitochondrial ATP-linked respiration, or a combination thereof) in iAstrocytes and/or neurons and/or oligodendrocytes derived from iNPCs which are in turn derived from skin cells of the subject, or in neurons derived directly from (or differentiated directly from) fibroblasts. In exemplary embodiments, the method comprises administering to the subject CuATSM in an amount effective to treat the subject. In exemplary aspects, the CuATSM is administered in an amount effective to reduce the level of basal mitochondrial respiration and/or level of mitochondrial ATP-linked respiration. In exemplary aspects, the CuATSM is administered in an amount effective to restore the level of basal mitochondrial respiration and/or level of mitochondrial ATP-linked respiration to the level of healthy subjects.

The present disclosure also provides methods of treating a subject with a seizure disorder. In exemplary embodiments, the method comprises administering to the subject CuATSM in an amount effective to treat the subject.

Further provided are methods of treating a subject with a neurodegenerative disorder associated with mitochondrial dysfunction, such as a neurodegenerative disorder associated with elevated levels of basal and/or ATP-linked respiration, in a subject. In exemplary embodiments, the method comprises administering to the subject CuATSM in an amount effective to treat the neurodegenerative disorder. Optionally, the subject does not suffer from ALS, Parkinson's Disease or Alzheimer's Disease.

Methods of treating a subject in need of improved or increased neuron survival, reduced mitochondrial basal and/or ATP-linked respiration, reduced cellular oxidative stress (e.g., oxidative stress linked to mitochondrial dysfunction), or a combination thereof, are provided. Methods of improving or increasing neuron survival, reducing mitochondrial basal and/or ATP-linked respiration, activating protective cellular signaling pathways. In exemplary embodiments, each method comprises administering to the subject CuATSM in an amount effective to treat the subject. In exemplary embodiments, the subject has elevated or dysfunctional levels of peroxynitrite and administration of CuATSM reduces the levels of peroxynitrite in the subjects in need thereof. Optionally, the subject does not suffer from ALS, Parkinson's Disease or Alzheimer's Disease.

In various aspects of the presently disclosed methods, CuATSM is administered to the subject once daily. In various instances, CuATSM is administered to the subject orally. In some aspects, CuATSM is formulated in a capsule or a powder for oral suspension. In other aspects, CuATSM is administered intravenously or systemically. In various instances, CuATSM is administered to the subject via the cerebrospinal fluid (CSF). In various aspects, CuATSM is administered at a dosage of at least or about 1 mg/day. In exemplary aspects, the dosage is at least or about 3 mg/day, at least or about 6 mg/day, at least or about 12 mg/day, at least or about 24 mg/day, at least or about 36 mg/day, at least or about 48 mg/day, at least or about 72 mg/day, or at least or about 100 mg/day. In exemplary aspects, the dosage is an amount effective to achieve CuATSM levels in plasma of a human comparable or equivalent to an ALS-mouse treated with 30 mg/kg/day. In exemplary instances, CuATSM is administered in an amount effective to reduce or restore the levels of basal and/or ATP-linked respiration in the induced astrocytes or neurons made from patient skin cells of the subject equal to or less than a control level. CuATSM in various aspects is administered in an amount effective to restore or reduce the levels of basal mitochondrial respiration in one or more of various cell types of the subject equal to or less than a control level. Optionally, the control level is a level of mitochondrial basal and/or ATP-linked respiration of a healthy, undiseased subject such as the level of basal and/or ATP-linked respiration in a comparable cell in a heathy, undiseased subject. In exemplary aspects, the subject does not have ALS, Parkinson's Disease or Alzheimer's Disease.

In other embodiments, the disclosure provides for compositions for treating a subject comprising a SCN2A mutation, a mutated SCN2A voltage-gated sodium channel protein, a SLC6A1 mutation, a SCN1A mutation or a mutation in IRF2BPL. In exemplary embodiments, the composition comprises copper-ATSM (CuATSM) in an amount effective to treat the subject.

Also provided herein are compositions for treating a subject with elevated levels of basal mitochondrial respiration, mitochondrial basal and/or ATP-linked respiration, or a combination. In exemplary aspects, the composition comprises copper-ATSM (CuATSM) in an amount effective to treat the subject. Optionally, the subject does not suffer from ALS, Parkinson's Disease or Alzheimer's Disease.

The present disclosure also provides compositions for treating a subject with mitochondrial changes (e.g., changes in mitochondrial function, relative to a control, e.g., elevated levels of basal mitochondrial respiration, elevated mitochondrial basal and/or ATP-linked respiration, or a combination thereof) in iAstrocytes and/or neurons and/or oligodendrocytes derived from iNPCs which are in turn derived from skin cells of the subject, or in neurons derived directly from (or differentiated directly from) fibroblasts. In exemplary embodiments, the composition comprises CuATSM in an amount effective to treat the subject. In exemplary aspects, the composition comprises CuATSM is an amount effective to reduce the level of basal mitochondrial respiration and/or level of mitochondrial basal and/or ATP-linked respiration. In exemplary aspects, the composition comprises CuATSM in an amount effective to restore the level of basal mitochondrial respiration and/or level of mitochondrial basal and/or ATP-linked respiration to the level of healthy subjects.

The present disclosure also provides compositions for treating a subject with a seizure disorder. In exemplary embodiments, the composition comprises CuATSM in an amount effective to treat the subject.

Further provided are compositions for treating a subject with a neurodegenerative disorder associated with mitochondrial dysfunction, such as a neurodegenerative disorder associated with elevated levels of mitochondrial basal and/or ATP-linked respiration, in a subject. In exemplary embodiments, the composition comprises CuATSM in an amount effective to treat the neurodegenerative disorder.

Compositions for improving survival of motor neurons or other neuronal cell types, or reducing mitochondrial basal and/or ATP-linked respiration, reducing mitochondrial oxidative stress (e.g., oxidative stress linked to mitochondrial dysfunction), or a combination thereof in a subject are provided. In exemplary embodiments, each composition comprises CuATSM in an amount effective to improve survival of motor neurons, reduce mitochondrial basal and/or ATP-linked respiration, and/or reduce mitochondrial oxidative stress in the subject. In exemplary embodiments, the subject has elevated or dysfunctional levels of peroxynitrite and administration of CuATSM reduces the levels of peroxynitrite in a subject in need thereof.

In various aspects of the present disclosure, compositions comprising CuATSM are formulated for administration to the subject once daily. In various instances, the composition is formulated for administration to the subject orally. In some aspects, the composition is formulated in a capsule or a powder for oral suspension. In other aspects, the composition is formulated for administration intravenously or systemically. In various instances, the composition is formulated for administration to the subject via the cerebrospinal fluid (CSF). In various aspects, the composition comprising CuATSM is in an amount effective to reduce the levels of basal and/or ATP-linked respiration in the astrocytes of the subject equal to or less than a control level (at least to the control level or less than the control level). In various aspects, the composition comprising CuATSM is in an amount effective to reduce the levels of basal and/or ATP-linked mitochondrial respiration in cells of the subject equal to or less than a control level (at least to the control level or less than the control level). In related aspects, the control level is a level of mitochondrial basal and/or ATP-linked respiration of a healthy, undiseased subject. In various aspects, the composition comprising is at a dosage of at least or about 1 mg/day. In exemplary aspects, the dosage is at least or about 3 mg/day, at least or about 6 mg/day, at least or about 12 mg/day, at least or about 24 mg/day, at least or about 36 mg/day, at least or about 48 mg/day, at least or about 72 mg/day, or at least or about 100 mg/day. In exemplary aspects, the dosage is an amount effective to achieve CuATSM levels in plasma of a human comparable or equivalent to an ALS-mouse treated with 30 mg/kg/day.

In other embodiments, the disclosure provides for uses of a CuATSM for the preparation of a medicament for treating a subject comprising a SCN2A mutation, a mutated SCN2A voltage-gated sodium channel protein, a SLC6A1 mutation, a SCN1A mutation or a mutation in IRF2BPL. In exemplary embodiments, the medicament CuATSM in an amount effective to treat the subject.

In other embodiments, the disclosure provides for uses of a copper-ATSM (CuATSM) for the preparation of a medicament for treating a subject with elevated levels of basal mitochondrial respiration, mitochondrial basal and/or ATP-linked respiration, or a combination. In exemplary aspects, the composition comprises copper-ATSM (CuATSM) in an amount effective to treat the subject.

Also provided herein are uses of a CuATSM for the preparation of a medicament for treating a subject with mitochondrial changes (e.g., changes in mitochondrial function, relative to a control, e.g., elevated levels of basal mitochondrial respiration, elevated mitochondrial basal and/or ATP-linked respiration, or a combination thereof) in iAstrocytes and/or neurons and/or oligodendrocytes derived from iNPCs which are in turn derived from skin cells of the subject, or in neurons derived directly from (or differentiated directly from) fibroblasts. In exemplary embodiments, the medicament comprises CuATSM in an amount effective to treat the subject. In exemplary aspects, the medicament comprises CuATSM an amount effective to reduce the level of basal mitochondrial respiration and/or level of mitochondrial basal and/or ATP-linked respiration. For example, the medicament comprising CuATSM is in an amount effective to reduce the levels of basal and/or ATP-linked mitochondrial respiration in cells of the subject equal to or less than a control level (at least to the control level or less than the control level). In exemplary aspects, the medicament comprises CuATSM in an amount effective to restore the level of basal mitochondrial respiration and/or level of mitochondrial basal and/or ATP-linked respiration to the level of healthy subjects.

The present disclosure also provides uses of a CuATSM for the preparation of a medicament for treating a subject with a seizure disorder. In exemplary embodiments, the medicament comprises CuATSM in an amount effective to treat the subject.

Further provided are uses of a CuATSM for the preparation of a medicament for treating a subject with a neurodegenerative disorder associated with mitochondrial dysfunction, such as a neurodegenerative disorder associated with elevated levels of basal and/or ATP-linked respiration, in a subject. In exemplary embodiments, the medicament comprises CuATSM in an amount effective to treat the neurodegenerative disorder. Optionally, the subject does not suffer from ALS, Parkinson's Disease or Alzheimer's Disease.

Uses of a CuATSM for the preparation of a medicament for improving or increasing neuron survival, reducing mitochondrial basal and/or ATP-linked respiration, reducing mitochondrial oxidative stress (e.g., oxidative stress linked to mitochondrial dysfunction), or a combination thereof, are provided. In exemplary embodiments, each medicament comprises CuATSM in an amount effective to improve survival of motor neurons, reduce mitochondrial basal and/or ATP-linked respiration, and/or reduce cellular oxidative stress in the subject. In exemplary embodiments, the subject has elevated or dysfunctional levels of peroxynitrite and administration of a medicament comprising CuATSM reduces the levels of peroxynitrite in the subjects in need thereof. Optionally, the subject does not suffer from ALS, Parkinson's Disease or Alzheimer's Disease.

Any of the disclosed compositions and medicaments are formulated to administer CuATSM to the subject once daily. In various instances, any of the disclosed compositions and medicaments are formulated to be administered to the subject orally. In some aspects, CuATSM is in a capsule or a powder for oral suspension. In other aspects, the disclosed compositions and medicaments are formulated to be administered intravenously or systemically. In various instances, the disclosed compositions and medicaments are formulated to be administered to the subject via the cerebrospinal fluid (CSF). In various aspects, the disclosed compositions and medicaments comprise CuATSM at a dosage of at least or about 1 mg/day. In exemplary aspects, the dosage is at least or about 3 mg/day, at least or about 6 mg/day, at least or about 12 mg/day, at least or about 24 mg/day, at least or about 36 mg/day, at least or about 48 mg/day, at least or about 72 mg/day, or at least or about 100 mg/day. In exemplary aspects, the dosage is an amount effective to achieve CuATSM levels in plasma of a human comparable or equivalent to an ALS-mouse treated with 30 mg/kg/day. In exemplary instances, the disclosed compositions and medicaments comprise CuATSM in an amount effective to reduce or restore the levels of mitochondrial basal and/or ATP-linked respiration in the astrocytes of the subject to a control level. For example, the compositions or medicaments comprise CuATSM is in an amount effective to reduce the levels of basal and/or ATP-linked mitochondrial respiration in cells of the subject equal to or less than a control level (at least to the control level or less than the control level). In various aspects, the disclosed compositions and medicaments comprise CuATSM in an amount effective to restore or reduce the levels of basal mitochondrial respiration in one or more of various cell types of the subject to a control level. Optionally, the control level is a level of mitochondrial basal and/or ATP-linked respiration of a healthy, undiseased subject such as the level of basal and/or ATP-linked respiration in a comparable cell in a heathy, undiseased subject. In exemplary aspects, the subject does not have ALS, Parkinson's Disease or Alzheimer's Disease.

In various aspects of the presently disclosed methods, compositions or uses, the subject comprises a SCN2A mutation, a mutated SCN2A voltage-gated sodium channel protein a SLC6A1 mutation, a SCN1A mutation or a mutation in IRF2BPL. In other aspects of the presently disclosed methods, compositions or uses, the subject comprises skin cells that can be reprogrammed into iNPCs that differentiate into iAstrocytes and/or neurons and/or oligodendrocytes which exhibit elevated levels of basal mitochondrial respiration, mitochondrial basal and/or ATP-linked respiration, or a combination thereof.

In various aspects of the presently disclosed methods, compositions or uses, skin cells from the subject can be reprogrammed into induced neuronal progenitor cells (iNPCs) that differentiate into astrocytes, wherein the astrocytes exhibit an increased energy state.

In various aspects of the presently disclosed methods, compositions or uses, the increased energy state is reflected by the increased oxygen consumption and increased lactate production or increased extracellular acidification rate, or a combination thereof of the astrocytes.

In various aspects of the presently disclosed methods, compositions or uses, the subject has a neurodegenerative or neurological disorder associated with mitochondrial dysfunction, optionally, a neurodegenerative disorder associated with elevated levels of mitochondrial basal and/or ATP-linked respiration. In other aspects of the presently disclosed methods, compositions or uses, wherein the subject has a seizure disorder.

In various aspects of the presently disclosed methods, compositions or uses, the subject has a channelopathy, neuronal hyper excitability, lysosomal storage disease (e.g., Pompe and Batten Disease forms (CLN1-13)), Facioscapulohumeral Muscular Dystrophy (FSHD), Dravet Syndrome (SCN1A), NEDAMSS (IRF2BPL), epilepsy and other seizure disorders, seizure disorders caused by SPATA5 mutations, seizures disorders caused by SMARCAL1 mutations, neurological disorders caused by KIF1A mutations, Huntington's disease, SMA with respiratory distress and Charcot-Marie-Tooth Disease 2S (CMT2S), Rett syndrome, Huntington's Disease, Fronto-temporal Dementia, and Multiple Sclerosis, epileptic encephalopathy or a combination thereof. In other aspects of the presently disclosed methods, compositions or uses, the subject does not have ALS.

The present disclosure also provides methods of identifying a subject who is responsive to CuATSM therapy. In exemplary embodiments, the method comprises analyzing iAstrocytes and/or neurons and/or oligodendrocytes generated from iNPCs derived from skin cells obtained from the subject for a SCN2A mutation or a mutated SCN2A voltage-gated sodium channel protein, a SLC6A1 mutation, a SCN1A mutation or an IRF2BPL mutation, wherein the subject is identified as a subject who is responsive to CuATSM therapy when the iAstrocytes and/or neurons and/or oligodendrocytes comprise a SCN2A mutation, a mutated SCN2A voltage-gated sodium channel protein, a SLC6A1 mutation, a SCN1A mutation or a mutation in IRF2BPL. In various aspects, the method further comprises obtaining skin cells from the subject. In various instances, the method further comprises generating induced neuronal progenitor cells (iNPCs) from skin cells obtained from the subject.

In exemplary aspects, the method further comprises differentiating iNPCs into iAstrocytes and/or neurons and/or oligodendrocytes. In exemplary instances, the skin cells obtained from the subject are used to grow primary skin fibroblasts. Optionally, a direct conversion method is used to produce iNPCs. Such methods are described in Meyer et al., PNAS 829-832 (2014)). In exemplary embodiments, the method comprises analyzing the level of mitochondrial activity or energy state of astrocytes generated from induced neuronal progenitor cells derived from skin cells obtained from the subject, wherein the subject is identified as a subject who is responsive to CuATSM therapy when the astrocytes exhibit elevated mitochondrial activity compared to astrocytes from a healthy subject. In various aspects, the method further comprises a step of obtaining skin cells from the subject. In various instances, the method further comprises a step of generating induced neuronal progenitor cells (iNPCs) from skin cells obtained from the subject. In exemplary aspects, the method further comprises differentiating iNPCs into astrocytes or neurons. Optionally, the skin cells obtained from the skin biopsies of the subject are used to grow primary skin fibroblasts. In various aspects, the mitochondrial activity is analyzed by measuring basal mitochondrial respiration, mitochondrial basal and/or ATP-linked respiration, or a combination thereof, of the astrocytes. In exemplary instances, the energy state is analyzed by measuring oxygen consumption and lactate production or extracellular acidification rate, or a combination thereof of the astrocytes.

Further provided herein are methods of treating, compositions for treating, and use of CuATSM for the preparation of a medicament for treating a subject in need thereof. In exemplary embodiments, the method compositions for treating, or use comprises identifying a subject who is responsive to CuATSM therapy in accordance with the presently disclosed identifying methods and administering CuATSM therapy to the identified subject. In exemplary embodiments, the method or use comprises (a) obtaining a skin cells via a skin biospy from the subject (b) generating iAstrocytes and/or neurons and/or oligodendrocytes from iNPCs derived from skin cells obtained from the subject or generating neurons derived directly from fibroblasts obtained from the subject, (b) analyzing the iAstrocytes and/or neurons and/or oligodendrocytes for a SCN2A mutation, a mutated SCN2A voltage-gated sodium channel protein, a SLC6A1 mutation, a SCN1A mutation or a mutation in IRF2BPL, and (c) administering CuATSM therapy when the iAstrocytes and/or neurons and/or oligodendrocytes from iNPCs derived from skin cells obtained from the subject or neurons derived directly from fibroblasts from the subject has a SCN2A mutation, a mutated SCN2A voltage-gated sodium channel protein, a SLC6A1 mutation, a SCN1A mutation or a mutation in IRF2BPL.

Also provided herein are methods of determining effectiveness of CuATSM therapy. In exemplary embodiments, the method comprises analyzing the level of mitochondrial activity of astrocytes generated from induced neuronal progenitor cells derived from skin cells obtained from the subject after administration of CuATSM, wherein a decrease in basal and/or ATP-linked respiration in the astrocytes, or a decrease in oxidative stress in the astrocytes, or increase in surviving neurons cultured on top of pretreated astrocytes as compared to astrocytes from the subject before administration of CuATSM is indicative of effective CuATSM therapy. Moreover, effectiveness of CuATSM therapy can also be measured using neurons derived from patient skin cells by measuring survival, differentiation efficiency and length of neurites with and without CuATSM treatment.

### BRIEF DESCRIPTION OF THE DRAWINGS

FIGS. 1A-1C demonstrate that CuATSM treatment of ALS astrocytes rescues motor neuron survival. FIG. 1A is a schematic of drug screen co-culture assay. FIG. 1B is a representative image of motor neurons following 3 days in co-culture. Astrocytes were treated during differentiation with CuATSM. Astrocytes were then seeded in a 96 well plate in the absence of CuATSM to form a monolayer. 24 hours later motor neurons were seeded on top of astrocyte monolayer and viability was determined following 3 or 4 days in culture. FIG. 1C is a quantification of motor neuron survival following co-culture. Here, ALS3 and ALS7 are identified as CuATSM patient nonresponders (dashed bars). Data was normalized to average motor neuron survival of healthy controls. Data represents a minimum of 3 independent experiments. Statistical analysis was performed using unpaired t-test to corresponding untreated controls.

FIGS. 2A-2E demonstrate ALS CuATSM responders have elevated basal and/or ATP-Linked Respiration and CoxIV activity. FIG. 2A is a representative image of iAstrocyte mitochondria labeled with 350 nM mitotracker red. FIG. 2B). iAstrocytes were seeded on a 24 well Seahorse plate for extracellular flux analysis and a representative rate graph is shown. Basal oxygen consumption (C) was measured at three time points followed by ATP synthase inhibition using oligomycin. The difference between basal respiration and oligomycin addition was used to calculate ATP linked respiration (D). Here, ALS3 and ALS7 are identified as CuATSM patient nonresponders (dashed bars) and do not have elevation in basal and/or ATP-linked respiration. FIG. 2E) Oxygen consumption was measured in the presence of ADP and tetramethyl-p-phenylenediamine, TMPD, on permeabilized iAstrocytes to measure complex IV activity. Data was collected for 4 time points and normalized to cell number within corresponding well. All ALS responders had elevation in CoxIV activity which is consistent with increased levels of basal and/or ATP-linked respiration. Data for FIGS. 2C and 2D was normalized to a preselected healthy control (Ctl1) that was run on every seahorse plate. Data for FIGS. 2B-2D represents a minimum of 3 independent experiments, data for FIG. 2E represents a minimum of 2 independent experiments. Statistical analysis was performed using one way ANOVA comparing the mean of each column to Ctl1.

FIGS. 3A-3F demonstrate CuATSM reduces mitochondrial activity, increases superoxide production and in some cases reduces oxidative stress. Basal (FIG. 3A) and/or ATP-Linked Respiration (FIG. 3B) of ALS iAstrocytes treated with and without CuATSM was calculated as described in FIG. 2B using the Seahorse. FIG. 3C) CoxIV activity assay was also measured on treated and untreated iAstrocytes using the seahorse as previously described in FIG. 2C. FIG. 3D) Representative live cell imaging of superoxide production and oxidative stress on treated and untreated iAstrocytes. Superoxide and oxidative stress was measure using cellular ROS/RNS assay and imaged using the InCell. Oxidative stress (FIG. 3E) and superoxide production (FIG. 3F) were quantified using automated image quantification. CuATSM treatment significantly reduced basal and ATP linked respiration in all patient lines. CoxIV activity was also significantly reduced in the all but one patient line (ALS1). Interestingly, superoxide production was increased in all iAstrocyte lines treated with CuATSM whereas oxidative stress was either not changed or reduced. CuATSM patient nonresponders (ALS3 and ALS7) are identified by dashed bars. Experiments (FIGS. 3A, 3B and 3D-3F) were run at least in triplicate, experiment C was run in duplicate. Statistical analysis for all experiments comparing treated and untreated individual patient lines was performed using student T-test.

FIGS. 4A-4D demonstrate that CuATSM did not impact NO levels in most ALS patient cell lines. FIG. 4A) Representative image of nitric oxide (NO) levels analyzed using ROS/RNS assay as previously described in FIG. 3C. FIG. 4B) Quantitation of nitric oxide production. CuATSM patient nonresponders (ALS3 and ALS7) are identified by dashed bars. FIG. 4C) iAstrocytes treated with and without CuATSM were lysed and analyzed by western blot. Immuoblots were stained for iNOS and GAPDH. FIG. 4D) Relative quantitation of iNOS levels in treated and untreated astrocytes normalized to the average of healthy untreated controls. While CuATSM resulted in some change in nitric oxide levels, these changes were not correlated to changes in iNOS protein levels following CuATSM treatment. Thus iNOS and nitric oxide production are not mediating the therapeutic effect of CuATSM. All experiments were run at least in triplicate. Statistical analysis for FIG. 4C comparing treated and untreated individual patient lines was performed using student T-test.

FIGS. 5A-5B demonstrate that SCN2A astrocytes have elevated basal and ATP-linked respiration. iAstrocytes were seeded on a 96 well Seahorse plate for extracellular flux analysis. Basal oxygen consumption (FIG. 5A) was measured at three time points followed by ATP synthase inhibition using oligomycin. The difference between basal respiration and oligomycin addition was used to calculate ATP linked respiration (FIG. 5B). This data suggests SCN2A astrocytes are potential CuATSM responders. All experiments were run at least in triplicate. Statistical analysis was run comparing untreated individual patient lines to control line using one way-ANOVA.

FIGS. 6A-6B demonstrate that CuATSM restores basal oxygen consumption and ATP-linked respiration in SCN2A astrocytes to levels comparable to healthy controls. Astrocytes treated with and without CuATSM were seeded on a 96 well Seahorse plate for extracellular flux analysis. Basal oxygen consumption (FIG. 6A) was measured at three time points followed by ATP synthase inhibition using oligomycin. The difference between basal respiration and oligomycin addition was used to calculate ATP linked respiration (FIG. 6B). This data demonstrates that CuATSM is able to restore basal and/or ATP-linked respiration in SCN2A astrocytes to healthy levels and that SCN2A astrocytes are CuATSM responders. All experiments were run at least in duplicate. Statistical analysis comparing treated and untreated individual patient lines was performed using student T-test.

FIG. 7 demonstrates that ATP linked respiration is not increased in ALS patient nonresponders, ALS3 (sALS) and ALS7 (C9ORF72). Data was obtained according to methodology described in FIG. 2B. All experiments were run at least in triplicate. Statistical analysis comparing untreated individual patient lines to healthy controls was performed using one way-ANOVA.

FIGS. 8A-8D demonstrate that CuATSM improves neuronal survival during chemical differentiating of SCN2A fibroblasts to neurons. FIG. 8A) Schematic protocol of iNeuron reprogramming from patient fibroblasts. Fibroblast cells were seeded on 12-well plates and differentiated for 7 days into iNeurons. iNeurons were fixed and imaged/immunostained for Tuj1 neuronal marker. FIG. 8B) Representative images of fibroblasts (before) and iNeurons after reprogramming. FIG. 8C) Representative bright field images of iNeurons following seven days of differentiation (scale bar=50 μm). On day five of differentiation, cells were treated with 0.01% of CuATSM as well as on day seven. FIG. 8D) Quantification of neuron survival percentage after seven days of differentiation. Tuj1 positive cells from three to five different images per line were counted and data was normalized to the average count of healthy control. FIG. 8E) Schematic of drug screen co-culture assay. Astrocytes were treated during differentiation with CuATSM. Astrocytes were then seeded in a 96 well plate in the absence of CuATSM to form a monolayer. 24 hours later mouse neurons were seeded on top of astrocyte monolayer and viability was determined following 3 days in culture. FIG. 8F) Representative image of WT GFP-neurons following 3 days in co-culture with patient iAstrocyte cells. Scale bar=100 μm. FIG. 8G) Quantification of neuron survival following co-culture indicates that CuATSM improved mouse neuron survival. Data was normalized to the average neuron survival of healthy controls and represents a minimum of 2 independent experiments. Statistical analysis was performed using unpaired t-test to corresponding untreated controls. Error bars represent SEM.

FIGS. 9A to 9C relate to methods of a study described herein. FIG. 9A is an illustration of how skin fibroblasts are converted to neuronal progenitor cells (from Kim et al., Curr Opin Neurobiol. 2012 October; 22(5): 778-784). FIG. 9B is an illustration of how neuronal progenitor cells are differentiated into iAstrocytes then used for co-culture with GFP+ mouse neurons. FIG. 9C is an illustration of how patient iAstrocytes are also analyzed for ALS markers including mitochondrial dysfunction using the Agilent Seahorse analyzer.

FIG. 10 is a graph of OCR plotted as a function of ECAR.

FIGS. 11A-11G. FIG. 11A is immunostaining of ALS iAstrocytes for P62 and BIP. FIGS. 13B-13C are graphs quantifying immunostaining of iAstrocyte for P62 an BIP adjusted in imageJ to set a threshold to limit detection in control cells. All cells with immunostaining that exceeded this threshold was counted blindly (p62) or using ImageJ automation (BIP) and normalized to the number of cells in the well to determine number of cells with elevated or aggregated p62 (FIG. 11B) or elevated BIP (FIG. 11C). Western blots were also performed to quantify the total amount of BIP (FIG. 11D-E) and SOD1 (FIG. 11F-G) in a cell lysate. Responders (solid diamonds) vs nonresponders (open diamonds) were compared for both immunofluorescences and western blot. Data indicates that BIP, p62 and SOD1 levels do not distinguish responders from nonresponders. Data represents a minimum of 3 independent experiments. Statistical analysis was performed using one way ANOVA comparing average controls (dotted line).

FIGS. 12A-12C are graphs and images showing cellular glycolysis and mitochondrial coupling is not significantly different between most patient lines. iAstrocytes were seeded on a 24 or 96 well Seahorse plate for extracellular flux analysis. Oxygen consumption rate (OCR) and extracellular acidification rate (ECAR) were used to calculate glycolytic protein efflux rate (glycoPER), which quantifies media acidification by lactate production. Representative ECAR rate graph is shown in FIG. 12A. Total cellular glycolysis (FIG. 12B) is measured following mitochondrial shutdown (difference between basal and AA/Rot injection). Percent mitochondrial coupling (FIG. 12C) was calculated using values from basal and/or ATP-linked respiration using data from FIG. 2. The data indicates that total cellular glycolysis and percent coupled mitochondria do not distinguish patient responders from nonresponders. Dashed bars indicate patient lines classified as nonresponders in co-culture assay. Dotted line represents average control values. Data represents a minimum of 3 independent experiments. Statistical analysis was performed using one way ANOVA comparing average controls.

FIGS. 13A-13B are graphs demonstrating that CuATSM enhances cellular glycolysis and reduces mitochondrial activity of iAstrocytes through uncoupling. The impact of CuATSM on mitochondrial activity and glycolysis was assessed using the Seahorse. iAstrocytes were seeded on a 24 or 96 well Seahorse plate for extracellular flux analysis. Oxygen consumption rate (OCR) and extracellular acidification rate (ECAR) were used to calculate glycolytic protein efflux rate (glycoPER), which quantifies media acidification by lactate production. Total cellular glycolysis (FIG. 13A) is measured following inhibition of mitochondrial electron transport chain. Mitochondrial coupling (FIG. 13B) was calculated using values for basal and/or ATP-linked respiration obtained from FIG. 3. Dashed bars indicate patient lines classified as nonresponders in co-culture assay. All cells lines showed an increase in glycolysis following CuATSM treatment. In addition, slight mitochondrial uncoupling was observed which may explain the previously observed reduction in mitochondrial activity. Dotted line represents average control values. Data for FIGS. 13A-13B represents a minimum of 3 independent experiments. Statistical analysis was performed using a student t-test comparing treated lines against corresponding untreated controls.

FIGS. 14A-14C are graphs demonstrating that CuATSM reduces mitochondrial activity to a healthy energy state. FIG. 14A) Energy map of healthy vs ALS iAstrocyte generated by plotting oxygen consumption rate (OCR) and total extracellular acidification rate (ECAR). FIG. 14B) Energy map of ALS iAstrocyte CuATSM responders before and after treatment. Dashed line in FIGS. 14A and 14B represents threshold for healthy mitochondrial activity. Energy maps indicate that elevation in mitochondrial energy state distinguishes responders from nonresponders and that CuATSM treatment restores this energy state to healthy levels. FIG. 14C) Representative image of CuATSM effect on electron transport chain (ETC), mitochondria energy and cellular metabolism. ALS nonresponders are not impacted by reduction in mitochondrial energy states as their baseline mitochondrial activity is not elevated.

FIGS. 15A-15F shows the metabolism of ALS patient astrocytes is highly distinct. iAstrocytes were seeded on a 96 well Seahorse plate for extracellular flux analysis. Rate graphs for mitochondrial dependency on glycolysis (FIG. 15A) fatty acid oxidation (FIG. 15B) and glutaminolysis (FIG. 15C) is shown. Mitochondrial dependency is calculated by measuring basal OCR for three time points followed by injections of pathway specific inhibitors (UK5099, etomoxir, BPTES). The difference in OCR following inhibitor addition determines mitochondrial fuel dependency on specific pathway tested. In this case, mitochondrial dependency on glycolysis (FIG. 15D), fatty acid oxidation (FIG. 16E) and glutamine (FIG. 15F) is calculated. The combined mitochondrial metabolic dependency profile is patient line specific and does not distinguish responders from nonresponders. However, elevation in mitochondrial dependency on glucose may differentiate nonresponders in this case. Nonresponders are identified in 15D-15F by striped bars. Dotted line represents average control values. Data represents a minimum of 3 independent experiments. Statistical analysis was performed using one way ANOVA comparing to the average controls (FIGS. 15D, 15E and 15F).

FIGS. 16A-16C demonstrate that CuATSM does not impact mitochondrial metabolic dependency. The effect of CuATSM on mitochondrial dependency on glutamine (FIG. 16A), fatty acid (FIG. 16B) and glucose (FIG. 16C) oxidation is determined using the Seahorse. Basal oxygen consumption (OCR) was measured for three time points followed by a pathway specific inhibitor (UK5099, etomoxir, BPTES). The difference in OCR following inhibitor addition determines mitochondrial fuel dependency on specific pathway tested. While most patient iAstrocytes have a change in mitochondrial dependency following CuATSM treatment, the changes do not distinguish responders from nonresponders. Dotted line represents average control values. Data represents a minimum of 3 independent experiments. Statistical analysis was performed using one way ANOVA comparing the average of controls.

FIG. 17 is a scheme showing the conversion of SCN2A hPSC cells into brain organoids.

FIGS. 18A-18E demonstrate that SCN2A patient brain organoids have an elevated expression of SCN2A (indicated by red dots on the expression map) that was reduced by CuATSM treatment. Cell types expressing SCN2A gene in untreated (FIGS. 18A-18B) and treated (FIGS. 18C-18D) organoids are shown using uniform manifold approximation and projection (UMAP). Nav1.2 staining (green) of the same patient iNeurons (FIG. 18E) confirm that SCN2A is upregulated in the patient (SCN2A-1) and that CuATSM downregulates this expression in the same patient.

FIGS. 19A-19B show that Metallothionine (MT1 and MT2) is upregulated following CuATSM treatment. FIG. 19A) Single Cell RNA seq analysis of dissociated organoids comparing S4 DMSO vs. S4 CuATSM treated brain organoids within the astrocytes cluster shows upregulation of MT1 and MT2. FIG. 19B shows SCN2A iAstrocytes treated with CuATSM during their differentiation and immunostained for Metallothionine (red) and nucleus (blue). Experiments were performed in duplicate.

FIGS. 20A-20C show that CuATSM treatment of IRF2BPL astrocytes rescues neuron survival in co-culture. FIG. 20A) Schematic of drug screen co-culture assay. FIG. 20B) Representative image of neurons following 3 days in co-culture. FIG. 20C) Quantification of neuron survival following co-culture show reduced survival with IRF2BPL astrocytes. CuATSM pretreatment of all patient iAstrocytes significantly increase neuron survival in co-culture. Data was normalized to average neuron survival of healthy controls. Data represents a minimum of 2 independent experiments. Statistical analysis was performed using unpaired t-test to corresponding untreated controls. Treatment with CuATSM indicates potential improvement in motor neuron survival for NEDAMSS patients (p<0.0001).

FIGS. 21A-21C demonstrate that CuATSM reduces the elevated basal and/or ATP-linked respiration in IRF2BPL astrocytes. FIG. 21A) Schematic image of the seahorse assay. The base oxygen consumption rate was measured (FIG. 21B). ATP linked respiration was measured following mitochondrial shutdown using oligomycin and was calculated by subtracting the oligo OCR from basal OCR (FIG. 21C). Both basal and/or ATP-linked respiration was elevated in three of the four patient lines tested. iAstrocytes pretreated with CuATSM (dashed bars on the graph) had significant reduction in basal and/or ATP-linked respiration (FIG. 21C). Data represents a minimum of 2 independent experiments.

FIGS. 22A-22B demonstrate that CuATSM restores mitochondrial activity of SLC6A1 astrocytes. FIG. 22A) Mitochondrial basal oxygen consumption was measured at three time points followed by ATP synthase inhibition using oligomycin. The difference between basal respiration (FIG. 22A) and oligomycin addition was used to calculate ATP linked respiration (FIG. 22B). SLC6A1 patient iAstrocytes have elevated basal and ATP linked respiration. CuATSM pretreatment (+) reduces basal and/or ATP-linked respiration to levels comparable to controls. Dotted line represents maximum average control values. Data represents a minimum of 1 independent experiment

### DETAILED DESCRIPTION

CuATSM Treatment

The present disclosure provides methods comprising administering to a subject copper-ATSM (CuATSM). Cu-ATSM is an orally bioavailable, blood-brain barrier (BBB) permeable complex. It has been used in cellular imaging experiments to selectively label hypoxic tissue via its susceptibility to reduction by oxygen-depleted mitochondria. Recently, Cu-ATSM has been reported to improve locomotor function and survival in a transgenic ALS mouse model by delivering copper specifically to cells in the spinal cords of mice producing misfolded SOD1 proteins, Copper chaperone for SOD (CCS) is presumed to utilize the copper from Cu-ATSM to prevent misfolding of the SOD1 protein. See, e.g., Vavere et al., Dalton Trans. 43, 4893-4902 (2007); Roberts et al., Journal of Neuroscience 34(23), 8021-8031 (2014); and Williams et al., Neurobiology of Disease 89, (2016). As used herein, the term “CuATSM” is synonymous with Cu^{II}(atsm) and refers to diacetyl-bis(4-methylthiosemicarbazonato) copperII or (SP-4-2)-[[2,2′-(1,2-dimethyl-1,2-ethanediylidene)bis[N-methylhydrazinecarbothioamidato-κN²,κS]](2-)]-copper, which has the structure of Formula I:

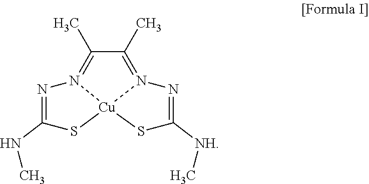

In exemplary aspects, the CuATSM is part of a pharmaceutical composition comprising CuATSM and a pharmaceutically acceptable carrier, diluent, or excipient. In exemplary aspects, the pharmaceutical compositions comprise a pharmaceutically acceptable carrier. As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopedia for use in animals, including humans.

The pharmaceutical composition in various aspects comprises any pharmaceutically acceptable ingredients, including, for example, acidifying agents, additives, adsorbents, aerosol propellants, air displacement agents, alkalizing agents, anticaking agents, anticoagulants, antimicrobial preservatives, antioxidants, antiseptics, bases, binders, buffering agents, chelating agents, coating agents, coloring agents, desiccants, detergents, diluents, disinfectants, disintegrants, dispersing agents, dissolution enhancing agents, dyes, emollients, emulsifying agents, emulsion stabilizers, fillers, film forming agents, flavor enhancers, flavoring agents, flow enhancers, gelling agents, granulating agents, humectants, lubricants, mucoadhesives, ointment bases, ointments, oleaginous vehicles, organic bases, pastille bases, pigments, plasticizers, polishing agents, preservatives, sequestering agents, skin penetrants, solubilizing agents, solvents, stabilizing agents, suppository bases, surface active agents, surfactants, suspending agents, sweetening agents, therapeutic agents, thickening agents, tonicity agents, toxicity agents, viscosity-increasing agents, water-absorbing agents, water-miscible cosolvents, water softeners, or wetting agents. See, e.g., the Handbook of Pharmaceutical Excipients, Third Edition, A. H. Kibbe (Pharmaceutical Press, London, U K, 2000), which is incorporated by reference in its entirety. Remington's Pharmaceutical Sciences, Sixteenth Edition, E. W. Martin (Mack Publishing Co., Easton, Pa., 1980), which is incorporated by reference in its entirety.

In exemplary aspects, the pharmaceutical composition comprises formulation materials that are nontoxic to recipients at the dosages and concentrations employed. In specific embodiments, pharmaceutical compositions comprising CuATSM and one or more pharmaceutically acceptable salts; polyols; surfactants; osmotic balancing agents; tonicity agents; anti-oxidants; antibiotics; antimycotics; bulking agents; lyoprotectants; anti-foaming agents; chelating agents; preservatives; colorants; analgesics; or additional pharmaceutical agents. In exemplary aspects, the pharmaceutical composition comprises one or more polyols and/or one or more surfactants, optionally, in addition to one or more excipients, including but not limited to, pharmaceutically acceptable salts; osmotic balancing agents (tonicity agents); anti-oxidants; antibiotics; antimycotics; bulking agents; lyoprotectants; anti-foaming agents; chelating agents; preservatives; colorants; and analgesics.

In certain embodiments, the pharmaceutical composition comprises formulation materials for modifying, maintaining or preserving, for example, the pH, osmolarity, viscosity, clarity, color, isotonicity, odor, sterility, stability, rate of dissolution or release, adsorption or penetration of the composition. In such embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; syrup and other carbohydrates (such as glucose, mannose or dextrins); sugar-free syrup; proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as bcnzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbatc, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants. See, REMINGTON'S PHARMACEUTICAL SCIENCES, 18″ Edition, (A. R. Genrmo, ed.), 1990, Mack Publishing Company.

The pharmaceutical compositions in various instances are formulated to achieve a physiologically compatible pH. In exemplary embodiments, the pH of the pharmaceutical composition is for example between about 4 or about 5 and about 8.0 or about 4.5 and about 7.5 or about 5.0 to about 7.5. In exemplary embodiments, the pH of the pharmaceutical composition is between 5.5 and 7.5.

The pharmaceutical composition may be administered to a subject via parenteral, nasal, oral, pulmonary, topical, vaginal, rectal or cerebrospinal fluid (CSF) administration. For example parenteral administration includes intrathecal, intracerebroventricular, intraparenchymal, intravenous, and a combination thereof. The following discussion on routes of administration is merely provided to illustrate exemplary embodiments and should not be construed as limiting the scope in any way.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The term, “parenteral” means not through the alimentary canal but by some other route such as subcutaneous, intramuscular, intraspinal, or intravenous. CuATSM in various instances is administered with a physiologically acceptable diluent in a pharmaceutical carrier, such as a sterile liquid or mixture of liquids, including water, saline, aqueous dextrose and related sugar solutions, syrup including sugar-free syrup, an alcohol, such as ethanol or hexadecyl alcohol, a glycol, such as propylene glycol or polyethylene glycol, dimethylsulfoxide, glycerol, ketals such as 2,2-dimethyl-I53-dioxolane-4-methanol, ethers, poly(ethyleneglycol) 400, oils, fatty acids, fatty acid esters or glycerides, or acetylated fatty acid glycerides with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils, which can be used in parenteral formulations include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters.

Suitable soaps for use in parenteral formulations include fatty alkali metal, ammonium, and triethanolamine salts, and suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylenepolypropylene copolymers, (d) amphoteric detergents such as, for example, alkyl-β-aminopropionates, and 2-alkyl-imidazoline quaternary ammonium salts, and (e) mixtures thereof.

The parenteral formulations in some embodiments contain from about 0.5% to about 25% by weight CuATSM in solution. Preservatives and buffers can be used. In order to minimize or eliminate irritation at the site of injection, such compositions can contain one or more nonionic surfactants having a hydrophile-lipophile balance (HLB) of from about 12 to about 17. The quantity of surfactant in such formulations will typically range from about 5% to about 15% by weight. Suitable surfactants include polyethylene glycol sorbitan fatty acid esters, such as sorbitan monooleate and the high molecular weight adducts of ethylene oxide with a hydrophobic base, formed by the condensation of propylene oxide with propylene glycol. The parenteral formulations in some aspects are presented in unit-dose or multi-dose sealed containers, such as ampoules and vials, and can be stored in a freeze-dried (lyophilized) condition requiring only the addition of the sterile liquid excipient, for example, water, for injections, immediately prior to use. Extemporaneous injection solutions and suspensions in some aspects are prepared from sterile powders, granules, and tablets of the kind previously described.

Injectable formulations are in accordance with the present disclosure. The requirements for effective pharmaceutical carriers for injectable compositions are well-known to those of ordinary skill in the art (see, e.g., Pharmaceutics and Pharmacy Practice, J. B. Lippincott Company, Philadelphia, Pa., Banker and Chalmers, eds., pages 238-250 (1982), and ASHP Handbook on Injectable Drugs, Toissel, 4th ed., pages 622-630 (1986)).

Formulations suitable for oral administration in some aspects comprise (a) liquid solutions, such as an effective amount of CuATSM dissolved in diluents, such as water, saline, syrups or orange juice; (b) capsules, sachets, tablets, lozenges, and troches, each containing a predetermined amount of CuATSM, as solids or granules; (c) powders; (d) suspensions in an appropriate liquid; and (e) suitable emulsions. Liquid formulations in some aspects include diluents, such as water and alcohols, for example, ethanol, benzyl alcohol, and the polyethylene alcohols, either with or without the addition of a pharmaceutically acceptable surfactant. Capsule forms can be of the ordinary hard- or soft-shelled gelatin type containing, for example, surfactants, lubricants, and inert fillers, such as lactose, sucrose, calcium phosphate, and corn starch. Tablet forms can include one or more of lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and other pharmacologically compatible excipients. Lozenge forms can comprise CuATSM in a flavor, usually sucrose and acacia or tragacanth, as well as pastilles comprising CuATSM in an inert base, such as gelatin and glycerin, or sucrose and acacia, emulsions, gels, and the like containing, in addition to, such excipients as are known in the art.

In various instances, CuATSM is administered to the subject orally. Optionally, CuATSM is formulated in a capsule or a powder in suspension. In various instances CuATSM is administered to the subject via the cerebrospinal fluid (CSF).

Dosages

CuATSM is believed to be useful in the methods of treating a subject comprising a SCN2A mutation, a mutated SCN2A voltage-gated sodium channel protein, a SLC6A1 mutation, a SCN1A mutation or a mutation in IRF2BPL, methods of treating a subject with elevated levels of basal mitochondrial respiration, mitochondrial basal and/or ATP-linked respiration, or a combination thereof, in iAstrocytes and/or neurons and/or oligodendrocytes of the subject, as well as other methods, as further described herein, including methods of treating a subject with a seizure disorder, methods of treating a subject with a neurodegenerative disorder associated with mitochondrial dysfunction, optionally, associated with elevated levels of mitochondrial basal and/or ATP-linked respiration, in a subject, and methods of treating a subject in need of increased or improved survival of motor neurons and other neuronal cell types, reduced mitochondrial basal and/or ATP-linked respiration, reduced cellular oxidative stress, reduced levels of peroxynitrite, or a combination thereof. For purposes of the present disclosure, the amount or dose of CuATSM administered should be sufficient to effect, e.g., a therapeutic or prophylactic response, in the subject or animal over a reasonable time frame. For example, the dose of CuATSM should be sufficient to treat a subject comprising a SCN2A mutation, a mutated SCN2A voltage-gated sodium channel protein, a SLC6A1 mutation, a SCN1A mutation or an IRF2BPL mutation in a period of from about 1 to 4 minutes, 1 to 4 hours or 1 to 4 weeks or longer, e.g., 5 to 20 or more weeks, from the time of administration. In certain embodiments, the time period could be even longer. The dose will be determined by the condition of the animal (e.g., human), as well as the body weight of the animal (e.g., human) to be treated.

Many assays for determining an administered dose are known in the art. For purposes herein, an assay, which comprises comparing the extent to which basal and/or ATP-linked respiration is restored to normal levels upon administration of a given dose of CuATSM to a mammal among a set of mammals, each set of which is given a different dose, could be used to determine a starting dose to be administered to a mammal. Methods of assaying basal and/or ATP-linked respiration are known in the art and described herein in EXAMPLES.

The dose of CuATSM also will be determined by the existence, nature and extent of any adverse side effects that might accompany the administration of CuATSM. Typically, the attending physician will decide the dosage with which to treat each individual patient, taking into consideration a variety of factors, such as age, body weight, general health, diet, sex, route of administration, and the severity of the condition being treated. By way of example and not intending to limit the present disclosure, the dosage is an amount effective to achieve CuATSM levels in plasma of a human comparable or equivalent to an ALS-mouse treated with 30 mg/kg/day. In various aspects, CuATSM is administered at a dosage of at least or about 1 mg/day. In exemplary aspects, the dosage is at least or about 3 mg/day, at least or about 6 mg/day, at least or about 12 mg/day, at least or about 24 mg/day, at least or about 36 mg/day, at least or about 48 mg/day, at least or about 72 mg/day, or at least or about 100 mg/day. For example, CuATSM is administered once a day after fasting. In exemplary instances, CuATSM is administered in an amount effective to restore the levels of mitochondrial basal and/or ATP-linked respiration in the astrocytes of the subject to close to a control level. CuATSM in various aspects is administered in an amount effective to restore the levels of basal mitochondrial respiration in astrocytes of the subject to near to a control level. Optionally, the control level is a level of mitochondrial basal and/or ATP-linked respiration of a healthy, undiseased subject, such as the basal and/or ATP-linked respiration of a comparable cell in a healthy, undiseased subject.

Controlled Release Formulations

In some embodiments, CuATSM is modified into a depot form, such that the manner in which CuATSM is released into the body to which it is administered is controlled with respect to time and location within the body (see, for example, U.S. Pat. No. 4,450,150). Depot forms of CuATSM can be, for example, an implantable composition comprising CuATSM and a porous or non-porous material, such as a polymer, wherein CuATSM is encapsulated by or diffused throughout the material and/or degradation of the non-porous material. The depot is then implanted into the desired location within the body of the subject and CuATSM is released from the implant at a predetermined rate.

The pharmaceutical composition comprising CuATSM in certain aspects is modified to have any type of in vivo release profile. In some aspects, the pharmaceutical composition is an immediate release, controlled release, sustained release, extended release, delayed release, or bi-phasic release formulation. Methods of formulating compounds for controlled release are known in the art. See, for example, Qian et al., J Pharm 374: 46-52 (2009) and International Patent Application Publication Nos. WO 2008/130158, WO2004/033036; WO2000/032218; and WO 1999/040942.

The CuATSM or pharmaceutical composition comprising the same may further comprise, for example, micelles or liposomes, or some other encapsulated form, or may be administered in an extended release form to provide a prolonged storage and/or delivery effect. The disclosed pharmaceutical formulations may be administered according to any regimen including, for example, daily (once per day, 2 times per day, 3 times per day, 4 times per day, 5 times per day, 6 times per day), six times a week, five times a week, four times a week, three times a week, twice a week, every two days, every three days, every four days, every five days, every six days, weekly, bi-weekly, every three weeks, monthly, or bi-monthly. In various aspects, CuATSM is administered to the subject once daily.

Combinations

The CuATSM may be administered alone or in combination with other therapeutic agents or therapy which aim to treat or prevent any of the subjects, diseases or medical conditions described herein. For example, CuATSM described herein may be co-administered with (simultaneously or sequentially) a medication for epilepsy or a seizure disorder (e.g., fosphenytoin, levetiracetam, lorazepam, midazolam, phenobarbital, phenytoin, propofol, and valproate). In some aspects, the CuATSM is administered in combination with (e.g., before, during or after) surgery, vagus nerve stimulation, and/or brain responsive neurostimulation. CuATSM could also be co-administered with gene therapy aiming to correct the mutation.

Subjects

In exemplary embodiments of the present disclosure, the subject is a mammal, including, but not limited to, mammals of the order Rodentia, such as mice and hamsters, and mammals of the order Logomorpha, such as rabbits, mammals from the order Carnivora, including Felines (cats) and Canines (dogs), mammals from the order Artiodactyla, including Bovines (cows) and Swines (pigs) or of the order Perssodactyla, including Equines (horses). In some aspects, the mammals are of the order Primates, Ceboids, or Simoids (monkeys) or of the order Anthropoids (humans and apes). In some aspects, the mammal is a human.

In exemplary aspects, the subject comprises a SCN2A mutation or a mutated SCN2A gene product, e.g., an SCN2A mRNA or SCN2A protein. The SCN2A gene is known in the art as the sodium voltage-gated channel alpha subunit 2 gene, and also as HBA; NAC2; BFIC3; BFIS3; BFNIS; HBSCI; EIEE11; HBSCII; Nav1.2; SCN2A1; SCN2A2; Na(v)1.2. The SCN2A gene sequence can be found at NCBI accession number NC_000002.125. The SCN2A gene encodes the alpha subunit of this voltage-gated sodium channel transmembrane glycoprotein. SCN2A is located in the human genome at ch. 2q24.3 and has 27 confirmed exons (suggested exons not confirmed=31). The sequences of the alpha subunit of isoforms 1 and 2 are listed in the NCBI database as follows.

| | Protein | SEQ ID | mRNA | SEQ ID |
|---|---|---|---|---|
| | Accession No. | NO: | Accession No. | NO. |
| Isoform 1 | NP_001035232.1 | 1 | NM_001040142.2 | 2 |
| Isoform 2 | NP_001035233.1 | 3 | NM_001040143.2 | 4 |

The SCN2A mutation may be any one of those SCN2A mutations described in the art. See, e.g., Shi et al., Brain Dev. 34(7): 541-545 (2012), Sanders et al., Trends in Neurosciences 41(7): 442-456. The SCN2A mutation may also be a new mutation that is currently not described. In exemplary aspects, the SCN2A mutation is a deletion, insertion, substitution mutation in the SCN2A gene. In various aspects, the SCN2A mutation is a missense mutation or a microduplication. In exemplary aspects, the SCN2A mutation is a nonsense mutation, synonymous mutation, silent mutation, neutral mutation, duplication mutation, splice mutation, or point mutation. Exemplary gene mutations are described in Mandieh and Rabban, Iran J Pedatr 23(4): 375-388 (2013). In some aspects, the gene mutation occurs in Exon 1, Exon 2, Exon 3, Exon 4, Exon 5, Exon 6, Exon 7, Exon 8, Exon 9, Exon 10, Exon 11, Exon 12, Exon 13, Exon 14, Exon 15, Exon 16, Exon 17, Exon 18, Exon 19, Exon 20, Exon 21, Exon 22, Exon 23, Exon 24, Exon 25, Exon 26, Exon 27 or a combination thereof (suggested exons not confirmed=31). In some aspects, the mutation could be in an intron, altering the splicing of the SCN2A mRNA leading to inclusion or exclusion of any of the exons described above or to the activation of a cryptic splice site that leads to the insertion of intronal sequences into the mRNA. In exemplary aspects, the mutation is any one of those listed in Table A. In exemplary instances, the mutated SCN2A gene product comprises a deletion, insertion, or substitution mutation in the SCN2A gene product. For instance, the mutated gene product may be a mutated SCN2A mRNA comprising a nucleic acid deletion, nucleic acid insertion, or nucleic acid substitution mutation relative to the wildtype SCN2A mRNA sequence. The SCN2A mRNA contains 27 exons (26 are coding) that encode a 2005 amino acid protein (called Nav1.2) (suggested exons not confirmed=31). In various aspects, the mutated gene product may be a mutated SCN2A protein comprising an amino acid deletion, amino acid insertion, or amino acid substitution relative to the wildtype. In various aspects, the mutation occurs in Domain I, Domain II, Domain III, or Domain IV of the protein encoded by the SCN2A gene. In various aspects, the mutation occurs in the extracellular domain, transmembrane domain, or intracellular domain of the protein. In various aspects SCN2A amino acid sequence, the mutation is a nonsense, canonical splice sites, frameshift insertion/deletions or large deletion in the first 1591 amino acids or the first 4773 nucleotides. In various aspects, the mutation is a nonsense, canonical splice sites, frameshift insertion/deletions or large deletion within the C-terminal portion of the amino acid sequence (e.g., a portion of the amino acid sequence starting at the amino acid at position 1592 to the C-terminal amino acid). In some aspects, the mutation is a protein truncation or a gene duplication. In exemplary aspects, the subject comprises a SCN2A-mediated disorder, such as any one of those described in Sanders et al., Trends in Neurosciences 41(7): 442-456, e.g., infantile epileptic encephalopathy (IEE), characterized by infantile-onset seizures, before 12 months of age, followed by neurodevelopmental delay; benign (familial) infantile seizures (BIS), characterized by infantile-onset seizures, before 12 months of age, that resolve by 2 years of age without overt long-term neuropsychiatric sequelae; and autism spectrum disorder/intellectual disability (ASD/ID), characterized by global developmental delay, particularly of social and language milestones. In various aspects, the SCN2A-mediated disorder is epileptic encephalopathy with choreoathetoid movements, benign infantile seizures with late-onset episodic ataxia, childhood-onset epileptic encephalopathy, and schizophrenia. In some aspects, SCN2A mutations could also lead to additional neurological phenotypes such as depression, avoidance of stimuli, reduced visual capacity.

TABLE A

| Chr2 | | | |
|---|---|---|---|
| Nucleotide (s) | Ref | Alt | Reference |
| 166164448 | G | A | 10 |
| 166152578 | A | G | 9 |
| 166170231 | G | A | 9 |
| 166170231 | G | A | 9 |
| 166183403 | — | A | 9 |
| 166201312 | G | A | 9 |
| 166245137 | A | T | 9 |
| 166152367 | G | A | 9 |
| 166201311 | C | T | 9 |
| 166201379 | C | A | 9 |
| 166210819 | G | T | 9 |
| 166231378 | T | C | 9 |
| 166234111 | C | T | 9 |
| 166198975 | G | A | 3 |
| 166198975 | G | A | 3 |
| 166245184 | C | A | 7 |
| 166231247 | T | C | 6 |
| 166234116 | A | G | 4 |
| 166179821- | CT | — | 1 |
| 166179822 | | | |
| 166172100 | — | A | 1 |
| 166201311 | C | T | 1 |
| 166231415 | G | A | 2 |
| 166243265 | C | T | 5 |
| 166187838 | A | G | 8 |

In exemplary aspects, the subject has an SCN2A-mediated disorder such as any of those described in Sanders et al., 2018, supra and Wolff et al., Brain 140(5):1316-1336 (2017). Mouse models for SCN2A mutations have been described, for example, see Kearney et al., Neuroscience. 2001; 102(2):307-17 (incorporated by reference in its entirety).

In various exemplary aspects, the subject comprises a SLC6A1 mutation or a mutated SLC6A1 gene product, e.g., a SLC6A1 mRNA or SLC6A1 protein. The SLC6A1 gene encodes a gamma-aminobutyric acid (GABA) transporter (GAT1) and alteration in GAT1 leads to aberrant tonic GABA inhibition, which results in absence seizures in GAT-1 knockout mice (Cope et al., Nat Med 2009; 15:1392-1398). The SLC6A1 gene sequence can be found at NCBI Gene ID: 6529 (NC_000003.12). SLC6A1 mutation has been associated with early onset absence epilepsy. Exemplary gene mutations include, but are not limited to, A288V, R44Q, L151Rfs*35, W193X, G457Hfs*10 or G234S. In related embodiments the subject may have epileptic encephalopathy. Mouse models for SLC6A1 mutations have been described, for example, see Madsen et al., J Pharmacol Exp Ther. 2011 July; 338(1): 214-219 and Xu et al., Biochem Biophys Res Commun. 2007 Sep. 21; 361(2):499-504 (incorporated by reference in their entirety). Any of these models may be used to investigate the methods or treatment disclosed herein.

The SLC6A1 mutation may be any one of those SLC6A1 mutations described in the art. See, e.g., Johannesen et al., Epilepsia. 2018 February; 59(2):389-402. The SLC6A1 mutation may also be a new mutation that is currently not described. In exemplary aspects, the SLC6A1 mutation is a deletion, insertion, substitution mutation in the SLC6A1 gene. In various aspects, the SLC6A1 mutation is a missense mutation or a microduplication. In exemplary aspects, the SLC6A1 mutation is a nonsense mutation, synonymous mutation, silent mutation, neutral mutation, duplication mutation, splice mutation, or point mutation. In some aspects, the gene mutation occurs in Exon 1, Exon 2, Exon 3, Exon 4, Exon 5, Exon 6, Exon 7, Exon 8, Exon 9, Exon 10, Exon 11, Exon 12, Exon 13, Exon 14, or Exon 15. In some aspects, the mutation could be in an intron, altering the splicing of the SLC6A1 mRNA leading to inclusion or exclusion of any of the exons described above or to the activation of a cryptic splice site that leads to the insertion of intronal sequences into the mRNA. In exemplary aspects, the mutation is any one of those listed in Table B.

TABLE B
SLC6A1 mutation

c.104dupA p.Lys36GluFsTer171
c.223G > A p.Gly75Arg
c.419A > G p.Tyr140Cys
c.434C > T p.Ser145Phe
c.578G > A p.Thp193Ter
c.695G > T, p.Gly232Val
c.809T > C p.Phe270Ser
C.863C > T p.Ala288Val
c.881_883del p.Phe294del
C.987C > A p.Cys329Ter
c.1024G > A p.Val342Met
C.1070C > T p.Ala357Val
c.1084g > a p.Gly362Arg
C.1155C > G p.Phe385Leu
c.1342A > T p.Lys448Ter
c.1369_1370 delGG Gly457HisFsTer10 de novo
C.1377C > G p.Ser459Arg
C.1531G > A p.Val511Met
C.1600C > T p.Gln534Ter
c.850-2A > G
c.6,1528-1G > C
3p25.3 del. including SLC6A11 and SLC6A1 (exon 1)

In various exemplary aspects, the subject comprises a SCN1A mutation or a mutated SCN1A gene product, e.g., a SCN1A mRNA or SCN1A protein. The SCN1A mutation may be any one of those SCN1A mutations described in the art (For example, see SCN1A mutations in Parihar et al., Journal of Human Genetics volume 58, pages 573-580 (2013), which is incorporated by reference in its entirety). The SCN1A gene encodes the alpha subunit of voltage-gated sodium channel Naᵥ1.1. This sodium channel is found on the surface of nerve cells, and is essential for the generation and transmission of electrical signals in the brain. The SCN1A gene is also known as GEFSP2, HBSCI, NAC1, Nav1.1, SCN1, sodium channel protein, brain I alpha subunit, sodium channel, voltage gated, type I alpha subunit, sodium channel, voltage-gated, type I, alpha, sodium channel, voltage-gated, type I, alpha polypeptide, or sodium channel, voltage-gated, type I, alpha subunit. The SCN1A gene sequence can be found at NCBI Gene ID: 6323 (NC_000002.12). SCN1A mutation has been associated with genetic (generalized) epilepsy with febrile seizures Plus (GEFS+) and Dravet syndrome (DS, severe myoclonic epilepsy of infancy) (Escayg and Goldin, Epilepsia. 2010 September; 51(9): 1650-1658). The SCN1A mutation may be any one of those SCN1A mutations described in the art. See, e.g., Parihar et al., Journal of Human Genetics 58, pages 573-580 (2013), which is incorporated by reference in its entirety. The SCN1A mutation may also be a new mutation that is currently not described. Mouse models for SCN1A mutations have been described, for example, see Kang et al., Epilepsia Open. 2019; 4(1): 164-169 and Miller et al., Genes Brain Behav. 2014 February; 13(2):163-72 (incorporated by reference in their entirety). Any of these models may be used to investigate the methods or treatments of disclosed herein.

In various exemplary aspects, the subject comprises a interferon regulatory factor 2 binding protein like (IRF2BPL) mutation or a mutated IRF2BPL gene product, e.g., an IRF2BPL mRNA or IRF2BPL protein. The IRF2BPL gene encodes a member of the IRF2BP family of transcriptional regulators (Marcogliese et al., Am J Hum Genet. 2018 Aug. 2; 103(2):245-260). The IRF2BPL gene is also known as C14orf4, EAP1, or NEDAMSS. The IRF2BPL gene sequence can be found at NCBI Gene ID: 64207 (NC_000014.9). The disease that has been associated with IRF2BPL mutations includes Neurodevelopmental Disorder With Regression, Abnormal Movements, Loss Of Speech, And Seizures (NEDAMSS). The IRF2BPL mutation may be any one of those IRF2BPL mutations described in the art. See, e.g., Marcogliese et al., Am J Hum Genet. 2018 Aug. 2; 103(2):245-260; Tran Mau-Them et al., Genetics in Medicine 21, pages 1008-1014(2019); Shelkowitz et al., Parkinsonism Relat Disord. 2019 62:239-241; Shelkowitz et al., Am J Med Genet A. 2019 November; 179(11):2263-2271. which are all incorporated by reference in their entirety. The IRF2BPL mutation may also be a new mutation that is currently not described. In exemplary aspects, the mutation is any one of those listed in Table C (IRF2BPL mutations described in Marcogliese et al., Am J Hum Genet. 2018 Aug. 2; 103(2):245-260 and Tran Mau-Them et al., Genetics in Medicine 21, pages 1008-1014(2019)).

TABLE C
IRF2BPL mutation

p.Glu172*
p.Gln127*
p.Arg188*
p.Pro372Arg
p.Lys418Asn
chr14: g.77493617G > C - NM_024496.3: c.519C > G

In exemplary aspects, the subject comprises skin cells which may be used to grow primary fibroblasts which may be reprogrammed (e.g., by way of a direct conversion method) to iNPCs, which in turn can differentiate into iAstrocytes and/or neurons and/or oligodendrocytes, and the iAstrocytes and/or neurons and/or oligodendrocytes so obtained in exemplary aspects exhibit elevated levels of basal mitochondrial respiration, mitochondrial basal and/or ATP-linked respiration, or a combination thereof. Methods of measuring levels of basal mitochondrial respiration and mitochondrial basal and/or ATP-linked respiration in cells are known in the art. See, e.g., the EXAMPLES herein.

In exemplary aspects, the subject has or exhibit mitochondrial changes (e.g., changes in mitochondrial function, relative to a control, e.g., elevated levels of basal mitochondrial respiration, elevated mitochondrial basal and/or ATP-linked respiration, or a combination thereof) or mitochondrial dysfunction as evidenced by iAstrocytes and/or neurons and/or oligodendrocytes derived from iNPCs which are in turn derived from skin cells of the subject, or in neurons derived directly from fibroblasts and/or neurons of the subject. In exemplary aspects, the mitochondrial changes are correctable or restorable to levels representative of normal healthy patients through CuATSM therapy. In various aspects, the subject is in need of improved or increased neuron survival, reduced basal and/or ATP-linked respiration, reduced oxidative stress (e.g., oxidative stress linked to mitochondrial dysfunction), or a combination thereof. In exemplary embodiments, the subject has elevated or dysfunctional levels of peroxynitrite and administration of CuATSM reduces the levels of peroxynitrite in the subjects in need thereof.

By “mitochondrial dysfunction” is meant a deviation from healthy individuals. Specifically, but not exclusively, CuATSM might be beneficial if the mitochondria of a patient show increased basal and/or ATP-linked respiration. In other cases, the mitochondria might show abnormal phenotype such as disturbance of the mitochondrial network or abnormal localization which results in mitochondrial dysfunction. This could also include changes in the cellular metabolism that can influence the mitochondrial activity including the electron transport chain.

The term “ATP-linked respiration or mitochondrial ATP-linked respiration” refers to the process in the mitochondria used to produce energy in the form of ATP. This occurs by sending electrons through an electron transport chain in the inner mitochondrial membrane, which produces a proton gradient across the membrane. The protons are then used by the ATP synthase to produce energy (ATP). This reaction consumes oxygen (ergo, respiration).

By “basal respiration” or “basal mitochondrial respiration” is meant the amount of oxygen consumed by the mitochondria within a cell without any chemically induced manipulation. It is the resting oxygen consumption rate of mitochondria within a given cell type.

By “survival of neurons” or “survival of motor neurons” is meant the ability of a neuron, e.g., motor neuron, to live despite potentially adverse conditions. Suitable methods of measuring neuron survival, e.g. motor neuron survival, are known in the art. In exemplary aspects, motor neuron survival is calculated 3-4 days following co-culture with human iAstrocytes and/or neurons and/or oligodendrocytes from patients or healthy individuals. In various instances, surviving motor neurons (defined as axon projections over 50 microns) are counted in each condition. The number of motor neurons remaining alive in each condition in various aspects is then normalized to the number of surviving motor neurons in non-diseased control lines. Survival is reported as a percent.

By “oxidative stress” is meant cumulative damage within an individual cell and/or body caused by free radicals that were not neutralized by cellular antioxidant processes. Oxidative stress can cause lipid peroxidation, DNA damage and oxidatively modified proteins. As a consequence, it can induce DNA mutations, damage cellular membranes and alter signaling pathways within the cell, ultimately leading to cellular death or dysfunction. In addition, oxidative damage in the central nervous system may impact cellular proliferation and remodeling, neural plasticity and neurogenesis with consequence on synaptic transmission (Salim, J Pharmacol Exp Ther 360(1): 201-205 (2017)). The impact of oxidative stress on neurons and neuronal support cells (such as astrocytes) leads to neurological phenotypes including seizures, behavioral abnormalities and neuronal death.

Suitable methods of measuring levels of peroxynitrite are known in the art. In exemplary aspects, the level of peroxynitrite is measured by measuring a bi-product, e.g., nitrotyrosine (see, e.g., Rios et al., Nitric Oxide, 3^{rd} ed., Elsevier, pages 271-288 (2017)). Also peroxynitrite reacts with tyrosine residues to form nitrotyrosine. Thus, in exemplary aspects, measurement of nitrated proteins is an indicator of the presence of peroxynitrite. In exemplary aspects, probes that detect peroxynitrite in live cells in vitro are used (Wu et al., Anal Chem 89(20) 10924-10931 (2017)).

In exemplary aspects, the subject has a seizure disorder. As used herein, the term “seizure disorder” is meant a medical condition characterized by episodes of uncontrolled electrical activity in the brain, thus producing symptoms that include two or more seizures. In various aspects, the seizure disorder is epilepsy (aka epileptic seizure disorder), simple partial seizure, benign rolandic epilepsy, catamenial epilepsy, atonic seizure, absence seizure, clonic seizure, tonic seizure, febrile seizure. In various aspects, the subject suffers from focal seizures, temporal lobe seizures, frontal lobe seizures, occipital lobe seizures, parietal lobe seizures, generalized seizures, absence seizures, myoclonic seizures, generalized convulsive seizures, generalized tonic-clonic seizures, symptomatic generalized epilepsy, progressive myoclonic epilepsy, reflex epilepsy. In various instances, the subject suffers from Ohtahara Syndrome, Benign Familial Neonatal seizures, infantile spasms, Dravet Syndrome (SCN1A), Rett Syndrome, Angelman Syndrome, Tuberous Sclerosis, Sturge-Weber Syndrome, Febrile Seizures, Landau-Kleffner Syndrome, Lennox-Gastaut Syndrome, Rasmussen Syndrome, Gelastic Epilepsy, Benign Rolandic Epilepsy, Benign Occipital Epilepsy, Childhood Absence Epilepsy, Juvenile Myoclonic epilepsy, neurodevelopmental disorder with regression, abnormal movements, loss of speech, and seizures (NEDAMSS) or epileptic encephalopathy.

In various aspects, the subject has a channelopathy, neuronal hyper excitability, lysosomal storage disease (e.g., Pompe and Batten Disease forms (CLN1-13)), Facioscapulohumeral Muscular Dystrophy (FSHD), seizure disorders caused by SPATA5 mutations, seizures disorders caused by SMARCAL1 mutations, neurological disorders caused by KIF1A mutations, SCN2A, NEDAMSS (IRF2BPL), SLC6A1, SCN1A, epilepsy and other seizure disorders, Huntington's disease, SMA with respiratory distress and Charcot-Marie-Tooth Disease 2S (CMT2S), Rett syndrome, Huntington's Disease, Fronto-temporal Dementia, and Multiple Sclerosis, or a combination thereof. In various instances, the subject has a neurodegenerative disorder associated with mitochondrial dysfunction, such as a neurodegenerative disorder associated with elevated levels of basal and/or ATP-linked respiration. In exemplary aspects, the subject does not have ALS. Optionally, the subject does not suffer from Parkinson's Disease or Alzheimer's Disease. In exemplary aspects, the subject has a disease in which oxidative stress plays a role. In various aspects, the subject has FSHD.

Neurodegenerative Disease

In exemplary aspects, the neurodegenerative disease is a disorder of the nervous system that involves mitochondrial dysfunction (e.g., elevated levels of basal mitochondrial respiration, elevated mitochondrial basal and/or ATP-linked respiration, or a combination thereof).

In exemplary aspects, the neurodegenerative disease is a neurodegenerative disorder associated with mitochondrial dysfunction, such as a neurodegenerative disorder with elevated levels of basal and/or ATP-linked respiration,

In various instances, the neurodegenerative disease is a disorder of the nervous system wherein cells of the nervous system comprise SCN2A mutations, mutated gene products of the SCN2A gene, the SCN1A gene, the IRF2BPL gene or SLC6A1 gene.

The neurodegenerative disease in various aspects is Alzheimer's disease, Parkinson's disease, Multiple Sclerosis, Amyotrophic Lateral Sclerosis (ALS), other demyelination related disorders, senile dementia, subcortical dementia, arteriosclerotic dementia, AIDS-associated dementia, or other dementias, a central nervous system cancer, traumatic brain injury, spinal cord injury, stroke or cerebral ischemia, cerebral vasculitis, epilepsy, Huntington's disease, Tourette's syndrome, Guillain Barre syndrome, Wilson disease, Pick's disease, neuroinflammatory disorders, encephalitis, encephalomyelitis or meningitis of viral, fungal or bacterial origin, or other central nervous system infections, prion diseases, cerebellar ataxias, cerebellar degeneration, spinocerebellar degeneration syndromes, Friedreichs ataxia, ataxia telangiectasia, spinal dysmyotrophy, progressive supranuclear palsy, dystonia, muscle spasticity, tremor, retinitis pigmentosa, striatonigral degeneration, mitochondrial encephalo-myopathies, neuronal ceroid lipofuscinosis, hepatic encephalopathies, renal encephalopathies, metabolic encephalopathies, toxin-induced encephalopathies, and radiation-induced brain damage.

In exemplary aspects, the neurodegenerative disease is not any of Alzheimer's disease, Parkinson's disease, and Amylotrophic Lateral Sclerosis (ALS).

Treatment

As used herein, the term “treat,” as well as words related thereto, do not necessarily imply 100% or complete treatment. Rather, there are varying degrees of treatment of which one of ordinary skill in the art recognizes as having a potential benefit or therapeutic effect. In this respect, the methods of treatment of the present disclosure can provide any amount or any level of treatment. Furthermore, the treatment provided by the method of the present disclosure can include treatment of one or more conditions or symptoms or signs of the cancer being treated. Also, the treatment provided by the methods of the present disclosure can encompass slowing the progression of the disease, disorder or medical condition aimed for treatment. For example, the methods can treat a neurodegenerative disease by virtue of enhancing cognitive and/or motor ability, reducing tremors, reducing muscle stiffness, improve balance, decrease amnesia, enhance speech ability, and the like. In exemplary aspects, the methods treat by way of delaying the onset or recurrence of the disease, disorder, or medical condition, or a sign or symptom thereof, by at least 1 day, 2 days, 4 days, 6 days, 8 days, 10 days, 15 days, 30 days, two months, 3 months, 4 months, 6 months, 1 year, 2 years, 3 years, 4 years, or more.

As used herein, the term “reduced” or “decreased” or synonyms thereof may not refer to a 100% or complete reduction or decrease. Rather, there are varying degrees of reduction or decrease of which one of ordinary skill in the art recognizes as having a potential benefit or therapeutic effect. In this respect, the CuATSM may reduce basal mitochondrial respiration or mitochondrial basal and/or ATP-linked respiration or reduce oxidative stress or reduce levels of peroxynitrite to any amount or level. In exemplary embodiments, the reduction provided by the methods of the present disclosure is at least or about a 10% reduction (e.g., at least or about a 20% reduction, at least or about a 30% reduction, at least or about a 40% reduction, at least or about a 50% reduction, at least or about a 60% reduction, at least or about a 70% reduction, at least or about a 80% reduction, at least or about a 90% reduction, at least or about a 95% reduction, at least or about a 98% reduction) relative to a control. In exemplary embodiments, the decrease provided by the methods of the present disclosure is at least or about a 10% decrease (e.g., at least or about a 20% decrease, at least or about a 30% decrease, at least or about a 40% decrease, at least or about a 50% decrease, at least or about a 60% decrease, at least or about a 70% decrease, at least or about a 80% decrease, at least or about a 90% decrease, at least or about a 95% decrease, at least or about a 98% decrease) relative to a control.

As used herein, the term “elevated” or “increased” or synonyms thereof may not refer to a 100% or complete elevation or increase. Rather, there are varying degrees of elevation or increase of which one of ordinary skill in the art recognizes as having a potential benefit or therapeutic effect. In this respect, the CuATSM may increase the overall survival of neurons (e.g., motor neurons) in a subject to any amount or level. In exemplary embodiments, the increase provided by the methods of the present disclosure is at least or about a 10% increase (e.g., at least or about a 20% increase, at least or about a 30% increase, at least or about a 40% increase, at least or about a 50% increase, at least or about a 60% increase, at least or about a 70% increase, at least or about a 80% increase, at least or about a 90% increase, at least or about a 95% increase, at least or about a 98% increase) relative to a control. In exemplary embodiments, the elevation provided by the methods of the present disclosure is at least or about a 10% elevation (e.g., at least or about a 20% elevation, at least or about a 30% elevation, at least or about a 40% elevation, at least or about a 50% elevation, at least or about a 60% elevation, at least or about a 70% elevation, at least or about a 80% elevation, at least or about a 90% elevation, at least or about a 95% elevation, at least or about a 98% elevation) relative to a control.

As used herein, the term “improve” or “enhance” or synonyms thereof may not refer to a 100% or complete improvement or enhancement. Rather, there are varying degrees of improvement or enhancement of which one of ordinary skill in the art recognizes as having a potential benefit or therapeutic effect. In this respect, the CuATSM may improve or enhance the survival of motor neurons to any amount or level. In exemplary embodiments, the improvement or enhancement provided by the methods of the present disclosure is at least or about a 10% improvement or enhancement (e.g., at least or about a 20% improvement or enhancement, at least or about a 30% improvement or enhancement, at least or about a 40% improvement or enhancement, at least or about a 50% improvement or enhancement, at least or about a 60% improvement or enhancement, at least or about a 70% improvement or enhancement, at least or about a 80% improvement or enhancement, at least or about a 90% improvement or enhancement, at least or about a 95% improvement or enhancement, at least or about a 98% improvement or enhancement) relative to a control.

Diagnostic Methods

The present disclosure also provides methods of identifying a subject who is responsive to CuATSM therapy. In exemplary embodiments, the method comprises analyzing iAstrocytes and/or neuron and/or oligodendrocytes generated from iNPCs derived from skin cells obtained from the subject or derived directly from fibroblasts obtained from the subject for a SCN2A mutation, a mutated SCN2A voltage-gated sodium channel protein, subjects with SCN1A mutations, IRF2BPL mutations or SLC6A1 mutation, wherein the subject is identified as a subject who is responsive to CuATSM therapy when the iAstrocytes and/or neurons and/or oligodendrocytes comprise a SCN2A mutation or a mutated SCN2A voltage-gated sodium channel protein, a SLC6A1 mutation, a SCN1A mutation or a mutation in IRF2BPL. In various aspects, the method further comprises obtaining skin cells from the subject. In various instances, the method further comprises generating induced neuronal progenitor cells (iNPCs) from skin cells obtained from the subject or generating neurons directly from fibroblasts obtained from the subject. In exemplary aspects, the method further comprises differentiating iNPCs into iAstrocytes and/or neurons and/or oligodendrocytes. In exemplary instances, the skin cells obtained from the subject are used to grow primary skin fibroblasts. Optionally, a direct conversion method is used to produce iNPCs. Such methods are described in Meyer et al., PNAS 829-832 (2014)).

In exemplary embodiments, the method of identifying a subject who is responsive to CuATSM therapy comprises analyzing the level of mitochondrial activity or energy state of astrocytes generated from induced neuronal progenitor cells derived from skin cells obtained from the subject, wherein the subject is identified as a subject who is responsive to CuATSM therapy when the astrocytes exhibit elevated mitochondrial activity compared to astrocytes from a healthy subject. In various aspects, the method further comprises a step of obtaining skin cells from the subject. In various instances, the method further comprises a step of generating induced neuronal progenitor cells (iNPCs) from skin cells obtained from the subject. In exemplary aspects, the method further comprises differentiating iNPCs into astrocytes or neurons. Optionally, the skin cells obtained from the subject are used to grow primary skin fibroblasts. In various aspects, the mitochondrial activity is analyzed by measuring basal mitochondrial respiration, mitochondrial basal and/or ATP-linked respiration, or a combination thereof, of the astrocytes. In exemplary instances, the energy state is analyzed by measuring oxygen consumption and lactate production or extracellular acidification rate, or a combination thereof of the astrocytes.

As used herein, the term “energy state” means a status of mitochondrial energy metabolism as described in Zhang and Zhang, Methods Mol Biol 1928: 353-363 (2019) and Zhang et al., Nat Protoc 7(6): doi:10.1038/nprot.2012.048. In exemplary aspects, the energy state of astrocytes is determined by measuring the oxygen consumption (OCR) and lactate production (extracellular acidification rate, ECAR) and then plotting the OCR as a function of ECAR to produce an energy map. Suitable methods of measuring OCR and ECAR are known in the art and include, for instance, the protocol described in Plitzko, B. and Loesgen, S. (2018). Bio-protocol 8(10): e2850. DOI: 10.21769/BioProtoc.2850; and Plitzko, B., Kaweesa, E. N. and Loesgen, S. (2017). J Biol Chem 292(51): 21102-21116; and Zhang and Zhang, Methods Mol Biol 1928: 353-363 (2019). In various instances, OCR is a measure of mitochondrial respiration and ECAR is a result of glycolysis.

Further provided herein are methods of treating a subject in need thereof. In exemplary embodiments, the method comprises identifying a subject who is responsive to CuATSM therapy in accordance with the presently disclosed identifying methods and administering CuATSM therapy to the identified subject. In exemplary embodiments, the method comprises (a) obtaining a skin cell sample from the subject (b) generating iAstrocytes and/or neurons and/or oligodendrocytes from iNPCs derived from skin cells obtained from the subject or generating neurons from fibroblast cells obtained from the subject, (b) analyzing the iAstrocytes and/or neurons and/or oligodendrocytes for a SCN2A mutation, a mutated SCN2A voltage-gated sodium channel protein, a SLC6A1 mutation, a SCN1A mutation or a mutation in IRF2BPL, and (c) administering CuATSM therapy when the iAstrocytes and/or neurons and/or oligodendrocytes from iNPCs derived from skin cells obtained from the subject and/or the neurons derived from fibroblasts obtained from the subject has a SCN2A mutation, a mutated SCN2A voltage-gated sodium channel protein, a SLC6A1 mutation, a SCN1A mutation or a mutation in IRF2BPL.

Methods of analyzing cells for a SCN2A mutation are known in the art. In exemplary embodiments, the analysis comprises conventional karyotyping, fluorescence in situ hybridization (FISH), comparative genomic hybridization (CGH), polymerase chain reaction (PCR), Multiplex PCR, Nested PCR, Real-time PCR, Restriction fragment length polymorphism (RFLP); Amplification refractory mutation system (ARMS); RT: Reverse transcriptase; Multiplex ligation-dependent probe amplification (MLPA); Denaturing Gradient Gel Electrophoresis (DGGE); Single Strand Conformational Polymorphism (SSCP); heteroduplex analysis; Chemical cleavage of mismatch (CCM); Protein truncation test (PTT); Oligonucleotide ligation assay (OLA), DNA microarray, DNA sequencing, Next Generation Sequencing (NGS) and the like. See, e.g., Mandieh and Rabbani, 2013, supra. Methods of analyzing cells for a mutated SCN2A voltage-gated sodium channel protein are known in the art. The methods in some aspects include an immunoassay using an antibody specific for the mutation. The immunoassay in various aspects is immunoprecipitation, Western blotting, ELISA, radioimmunoassay, and the like.

The sample in various aspects comprises a skin biopsy, e.g., a skin punch. In various aspects, the skin biopsy is used to grow skin cells such as primary skin fibroblasts.

Also provided herein are methods of determining effectiveness of CuATSM therapy. In exemplary embodiments, the method comprises analyzing the level of mitochondrial activity of astrocytes generated from induced neuronal progenitor cells derived from skin cells obtained from the subject after administration of CuATSM, wherein a decrease in basal and/or ATP-linked respiration in the astrocytes, or a decrease in oxidative stress in the astrocytes, or increase in surviving neurons cultured on top of pretreated astrocytes as compared to astrocytes from the subject before administration of CuATSM is indicative of effective CuATSM therapy. In various instances, the method further comprises a step of generating induced neuronal progenitor cells (iNPCs) from skin cells obtained from the subject. In exemplary aspects, the method further comprises differentiating iNPCs into astrocytes or neurons. Optionally, the skin cells obtained from the subject are used to grow primary skin fibroblasts. In various aspects, the mitochondrial activity is analyzed by measuring basal mitochondrial respiration, mitochondrial basal and/or ATP-linked respiration, or a combination thereof, of the astrocytes. In various aspects, effectiveness of CuATSM therapy can also be measured using neurons derived from patient skin cells by measuring survival, differentiation efficiency and length of neurites with and without CuATSM treatment.

The following examples are given merely to illustrate the present invention and not in any way to limit its scope.

### EXAMPLES

### Example 1

This example describes the materials and methods used in the experiments described in Examples 2-6.

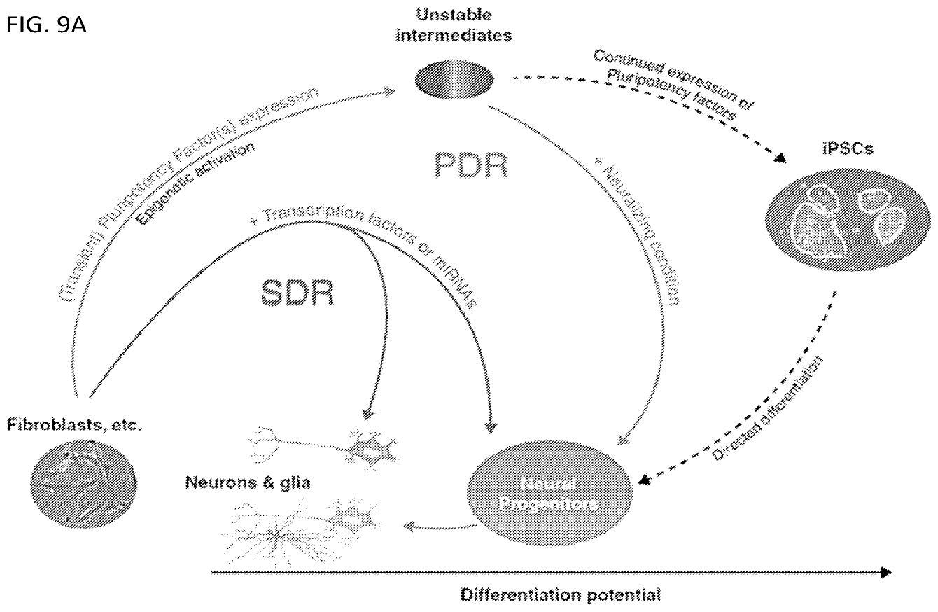

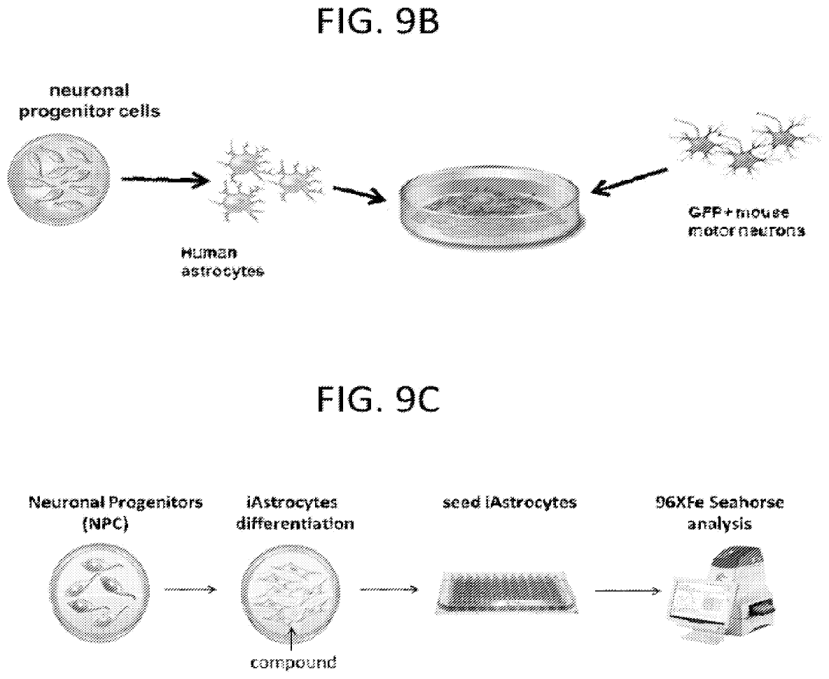

Direct conversion: Patient fibroblasts were converted to induced neuronal progenitor cells (iNPCs) as previously described (Meyer 2014, supra and as demonstrated in FIG. 9A, green path). Neuronal progenitors cells were cultured on fibronectin coated dishes in NPC media (DMEM/F12 media containing 1% N2 supplement (Life Technologies), 1% B27 and 20 ng/ml fibroblast growth factor-2) until confluent. Astrocytes were differentiated by seeding a small quantity of NPCs on another fibronectin coated dish in astrocyte inducing media (DMEM media containing 0.2% N2 and 10% FBS). Cells were treated with CuATSM daily, beginning day 2 of differentiation. Five days post differentiation, induced astrocytes were seeded either into a 96 well (10,000 cells/well), 384 well (2,500 cells/well), a 24 well seahorse plate (20,000 cells/well) or a 96 well seahorse plate (25,000 cells/well treated, 12,500 cells/well untreated).

Co-culture: Stem cells from HB9:GFP+ mouse embryos were cultured as described previously (Meyer 2014). Embryonic bodies (EB) were cultured in EB differentiation media (knockout DMEM/F12, 10% knockout serum replacement, 1% N2, 0.5% L-glutamine, 0.5% glucose, and 0.0016% 2-mercaptoethanol) with smoothen agonist and retinoic acid freshly added starting day 2 of differentiation. EBs were dissociated with papain as previously described (Meyer 2014) and sorted. GFP+ motor neurons were seeded on top of patient iAstrocytes in a 96 well plate (10,000 cells per well) or 384 well (1,000 cells per well) as demonstrated in FIG. 9B. Co-cultures were imaged with InCell 6000 (GE Healthcare) for up to four days. Motor neurons with neurite outgrowth of greater than 50 um were counted as alive. Data was normalized to healthy controls.

Mitochondrial imaging: Induced astrocytes were seeded on a black clear bottom plate 24 hours later cells were treated with 350 Nm mitotracker red and incubated at 37 in 5% CO2 for 30 minutes. Cells were imaged using a Nikon microscope. Alternatively, iAstrocytes were seeded on glass cover slips or plastic chamber slides and immunostained for complex IV (CoxIV). Immunostains were imaged using an Nikon microscope.

Mitochondrial function: Induced astrocytes were seeded on 24 well seahorse plates in quadruplicate or a 96 well seahorse plate in quintuplicate as summarized in FIG. 9C. Twenty-four hours later media was replaced with seahorse base media containing 10 Mm glucose and 2 Mm glutamine. Oligomycin (1 Um), FCCP (5 Um) and antimycin A (10 Um) were injected separately to evaluate oxygen consumption following inhibition of the ATPase synthase, mitochondrial uncoupling and total shutdown of the electron transport chain. Alternatively, iAstrocyte media was replaced with a mitochondrial buffer (220 Mm mannitol, 70 Mm sucrose, 10 Mm KH2PO4, 5 Mm Hepes, 1 Mm EGTA and 0.2% Fatty acid free BSA) to measure complex IV dependent oxygen consumption. CoxIV activity was induced using 0.5 Mm TMPD, 2 Mm Ascorbate, 1 Mm ADP, 10 Um antimycin A, 10 Mm azide and 1.1 nM seahorse XF membrane permeabilizer. Oxygen consumption was measured in both assays using the Seahorse XF and Seahorse XFe.

Western blotting: iAstrocyte pellets were lysed in RIPA or IP buffer and with sonication or freeze thaw respectively. 50 ug of protein was loaded into precast gels and separated by electrophoresis. Protein was then transferred to a PVDF membrane, blocked with odyssey blocking buffer (Licor) and incubated with primary antibody overnight. The following day membranes were washed and incubated with Licor secondary antibodies for one hour at room temperature. Membranes were imaged using Odyssey CLx.

Superoxide, nitric oxide and oxidative stress in live cells was measured using a cellular ROS/RNS detection assay (Abcam). Live cell staining was imaged using the InCell and total fluorescent intensity and cell counting was quantified using a computer code developed for this purpose.

Neuronal differentiation was performed as previously described (Hu et. al. 2016). Briefly neurons were differentiated in a chemically defined media for 5-7 days. At 5-7 days neurons were imaged and the number of cells expressing neuronal markers such as Tuj1 and Map2 and contained neurite outgrowths greater than two times the soma were counted.

### Example 2

This example demonstrates that CuATSM increases motor neuron survival on both sporadic and familial ALS astrocytes.

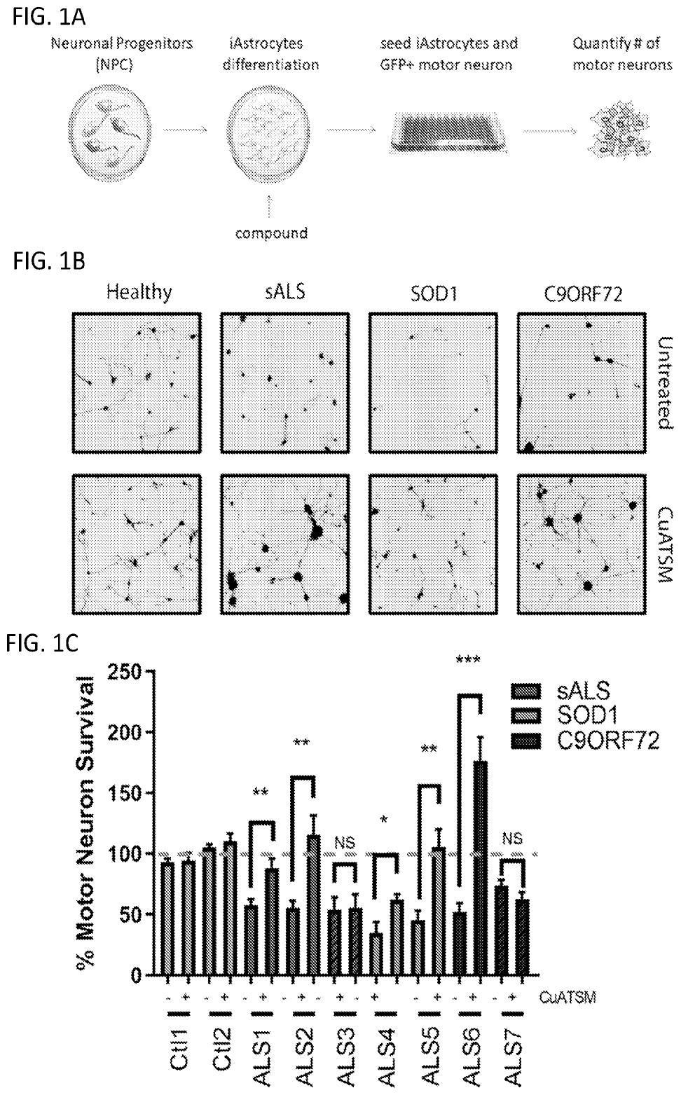

A previously established direct conversion co-culture system was utilized to assess the therapeutic potential of CuATSM in the treatment of ALS (Meyer et al., PNAS, 111 (2) 829-832(2014)). Induced astrocytes (iAs) were treated daily during differentiation with 1 Um CuATSM. Following differentiation astrocytes were co-cultured with GFP+ motor neurons to assess motor neuron viability (FIG. 1A and FIG. 9B). Three days following co-cultures motor neurons were imaged (FIG. 1B) and motor neuron survival was calculated (FIG. 1C). Of the patient lines tested (3 sALS, 2 mutant SOD1, and 2 C9ORF72 patients), all lines except for one sALS (ALS3) and one C9ORF72 (ALS7) patient had increased motor neuron survival following continuous treatment with CuATSM (FIG. 1). Using the results of this assay, patients were classified as CuATSM responders, and nonresponders and sought to identify a phenotype common amongst responders that was absent in nonresponders.

### Example 3

This example demonstrates that ALS astrocytes that respond to CuATSM treatment have specific mitochondrial activity.

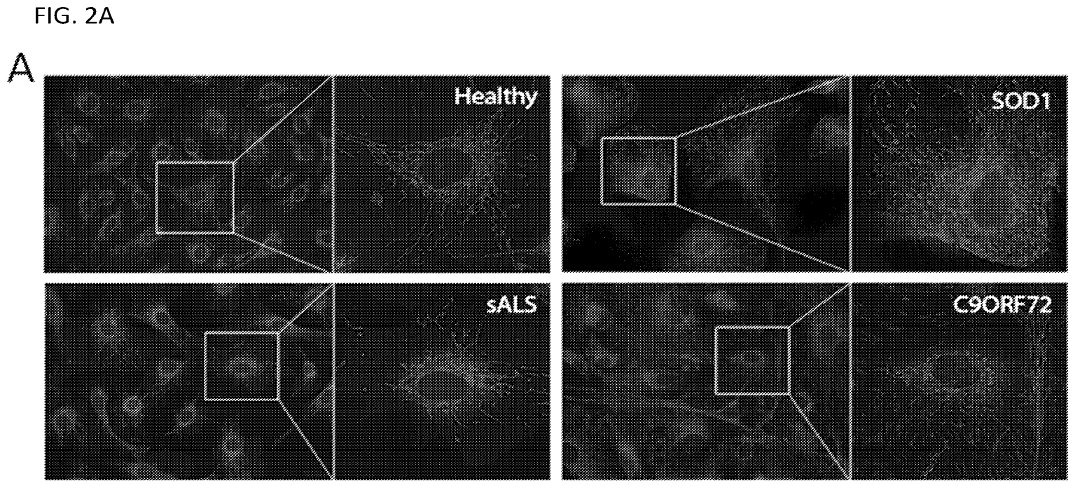

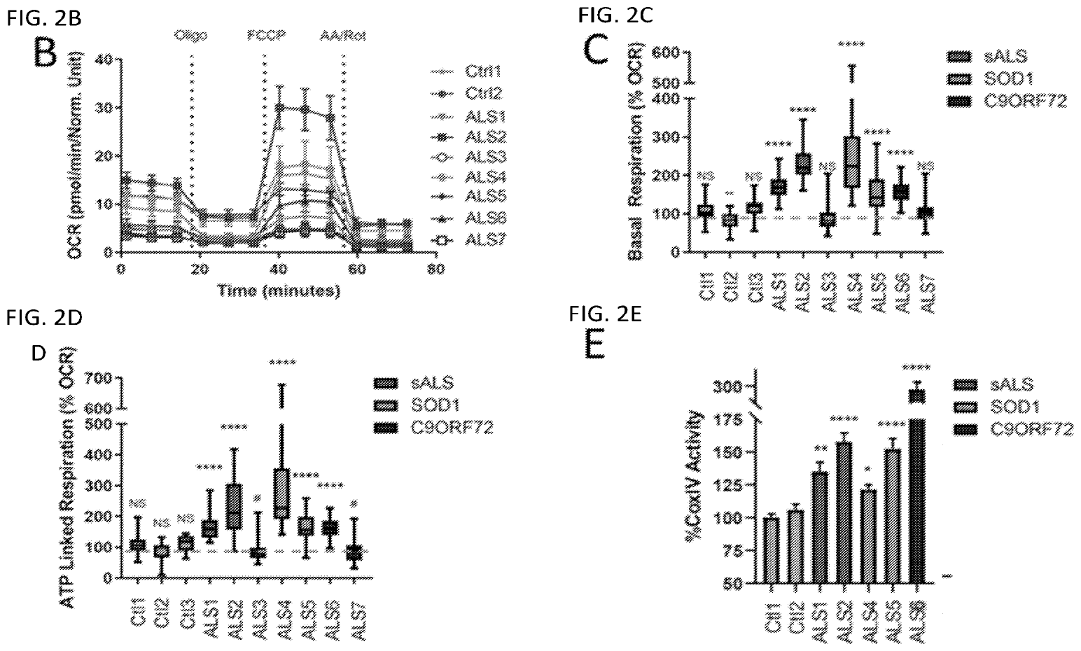

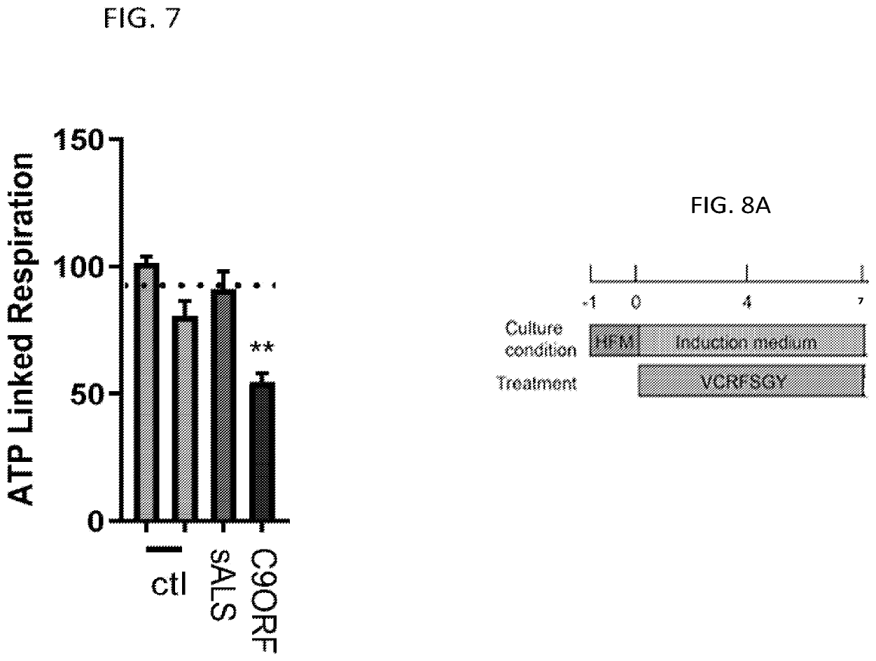

Post mortem patient tissue as well as human in vitro models have established that most human samples do not have an ALS phenotype that directly correlate to the transgenic mouse model. Most patient samples have some, but not all, phenotypic ALS characteristics. One of the more common markers observed is mitochondrial dysfunction (Smith et al., Neurosci Lett. 2019 Sep. 25; 710:132933). In fact, live cell imaging of mitochondria in ALS iAstrocytes indicate highly variable mitochondrial morphology between patient lines. As expected, the control lines have large, intact tubular mitochondrial networks (FIG. 2A). Whereas the ALS lines have reduced mitochondrial network size and variable mitochondrial rounding between patients (FIG. 2A). Based on these observations it was sought to evaluate the functionality of the mitochondria within the patient lines by measuring oxygen consumption following a mitochondrial stress test (representative schematic in FIG. 9C). FIG. 2B shows a representative rate graph from these experiments. The findings of this mitochondrial stress test indicated that all patient responders had increased levels of basal (FIG. 2C) and ATP linked respiration (FIG. 2D), whereas the nonresponders did not (FIG. 2C-D and FIG. 7). This suggests increased activity of the electron transport chain. To confirm these findings, complex IV activity was measured in the patient responders. A significant increased complex IV activity was found in comparison to the healthy controls (FIG. 2E). All ALS responders had elevation in CoxIV activity which is consistent with increased levels of ATP-linked respiration. Combined, these findings suggest that increased activity basal and ATP-linked respiration, in addition to elevation in CoxIV activity, may distinguishes patient CuATSM responders from nonresponders.

### Example 4

This example demonstrates CuATSM restores mitochondrial activity to healthy control levels.

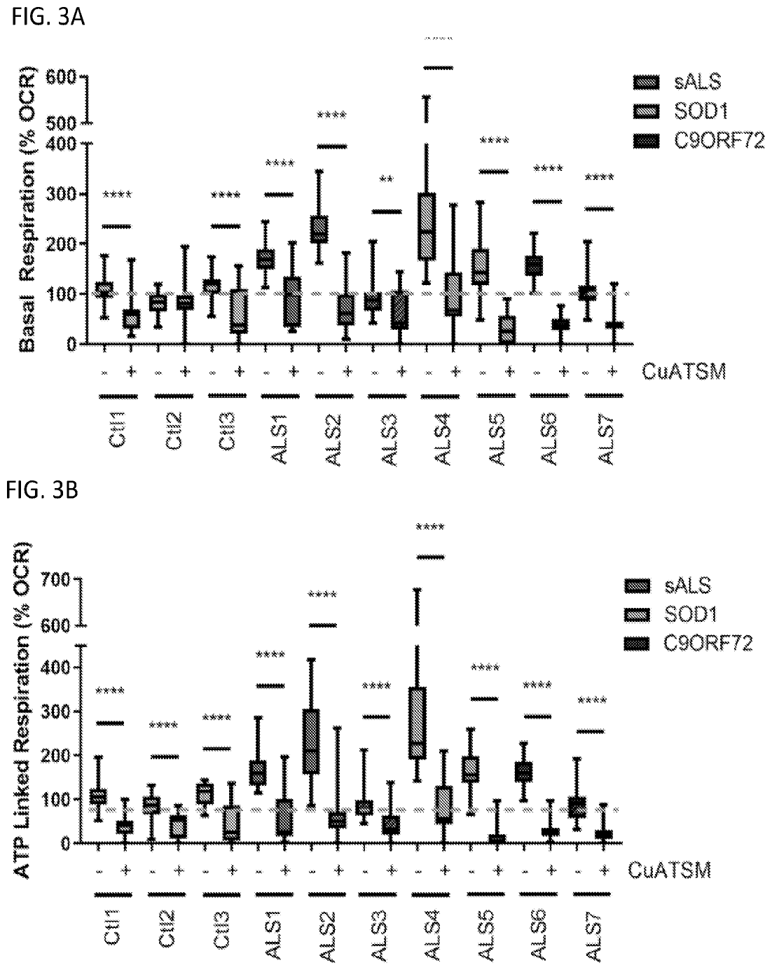

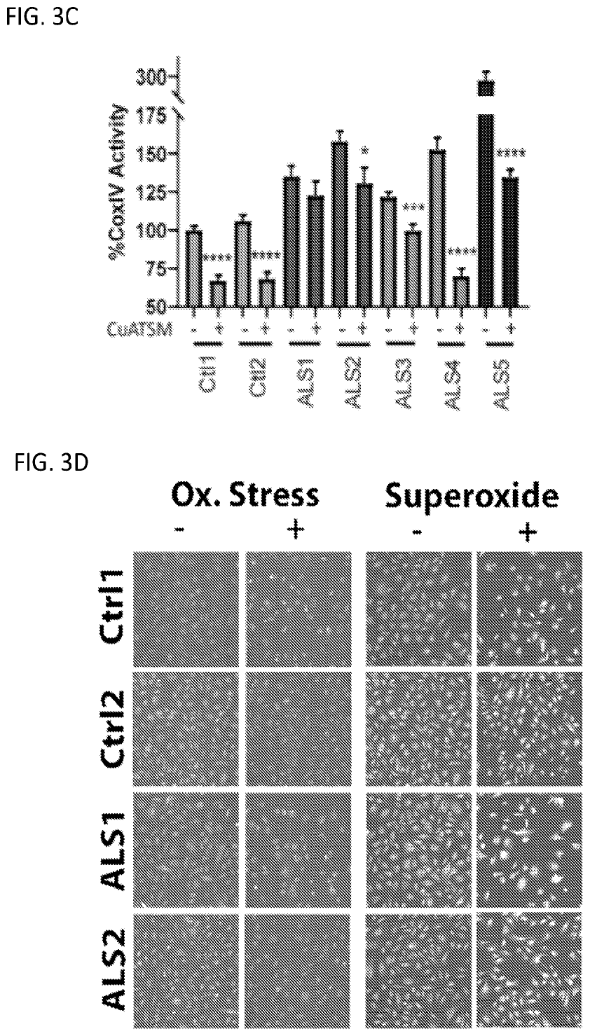

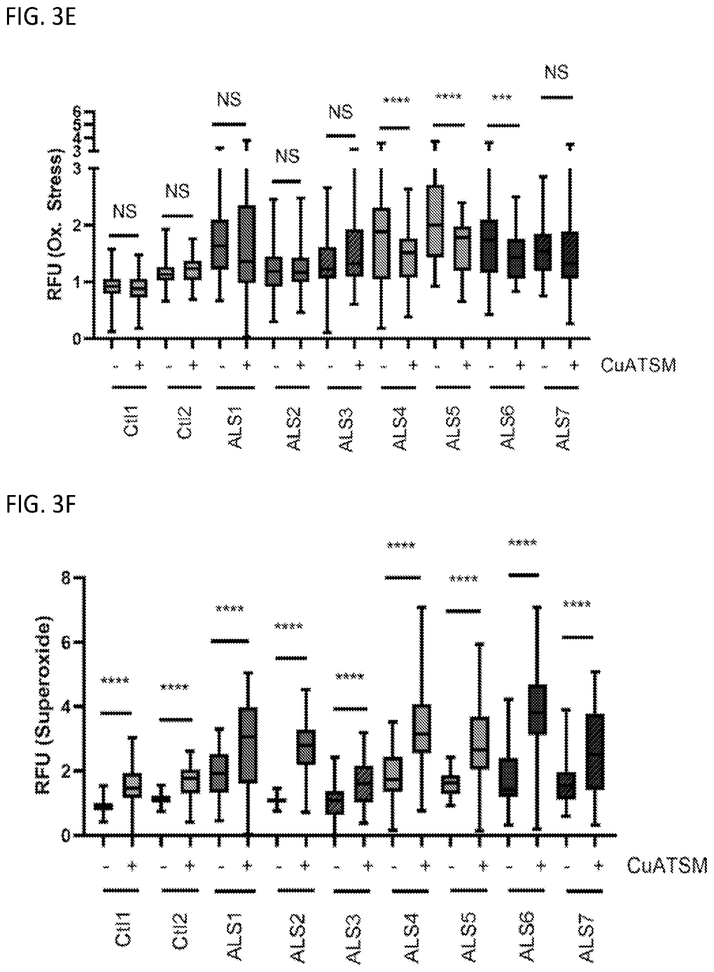

Given that CuATSM responders have elevation in basal and/or ATP-linked respiration as well as complex IV activity, it was sought to determine if CuATSM treatment can restore mitochondrial activity to normal range. To do so, the basal and/or ATP-linked respiration of treated and untreated astrocytes were compared. It was found that CuATSM treatment significantly reduced basal and/or ATP-linked respiration of ALS patient lines to respiration levels at or below the healthy untreated controls (FIGS. 3A and 3B). Further investigation into the effects of CuATSM on complex IV activity confirms reduction of the electron transport chain activity (FIG. 3C). However, CuATSM does not fully reduce CoxIV activity to healthy control levels which may be explained by uninvestigated copper mediated cellular signaling. The overall mitochondrial basal and ATP linked respiration is restored to healthy levels, and patient iAstrocytes are no longer toxic to neurons. Given that mitochondrial dysfunction is a major source of superoxide production, a cellular ROS/RNS detection assay was used to determine if CuATSM is having an effect on reducing oxidative stress. While oxidative stress was not significantly reduced in all patient lines, it was reduced in some lines (FIGS. 3D-3E). CuATSM treatment significantly reduced basal and ATP linked respiration in all patient lines. CoxIV activity was also significantly reduced in the all but one patient line (ALS1). Of particular importance, CuATSM significantly reduced oxidative stress in SOD1 mutant patient lines (ALS6 and ALS7) as previously described (Williams et al., Neurobiology of Disease 89, (2016)). In addition, there was reduction in oxidative stress in one C9ORF72 patient line (ALS8, FIGS. 3D-3E). Addition of CuATSM significantly increased the levels of superoxide production following CuATSM treatment (FIGS. 3D and 3F). Interestingly, superoxide production was increased in all iAstrocyte lines treated with CuATSM whereas oxidative stress was either not changed or reduced. This elevation of superoxide production, along with no increase in oxidative stress, may result in superoxide mediate cellular signaling, that is separate from the therapeutic effect of CuATSM.

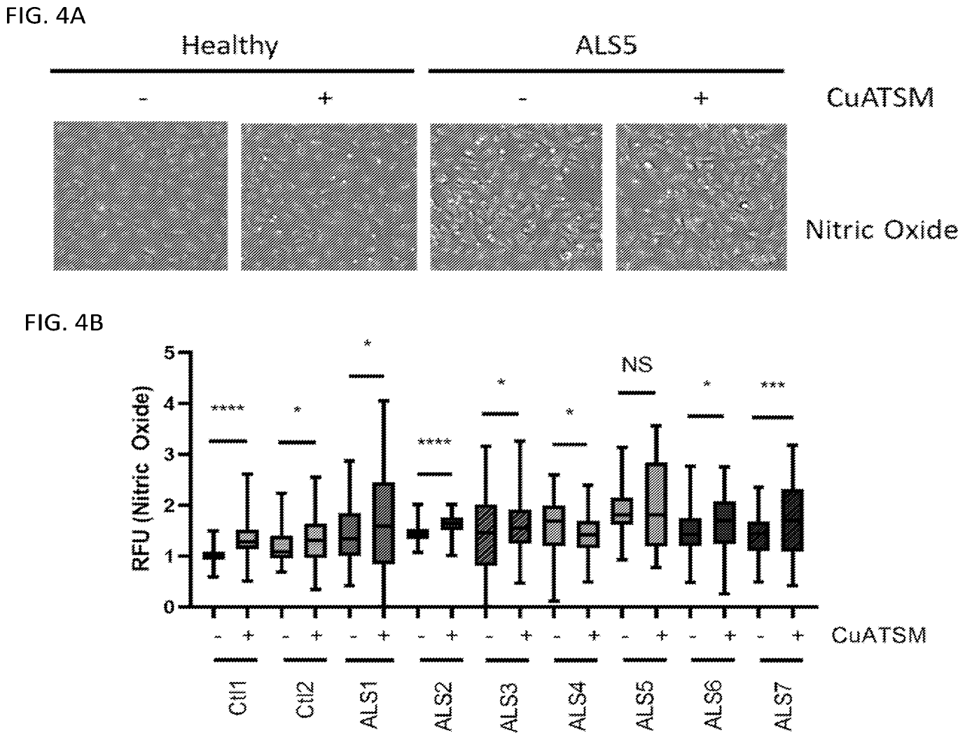

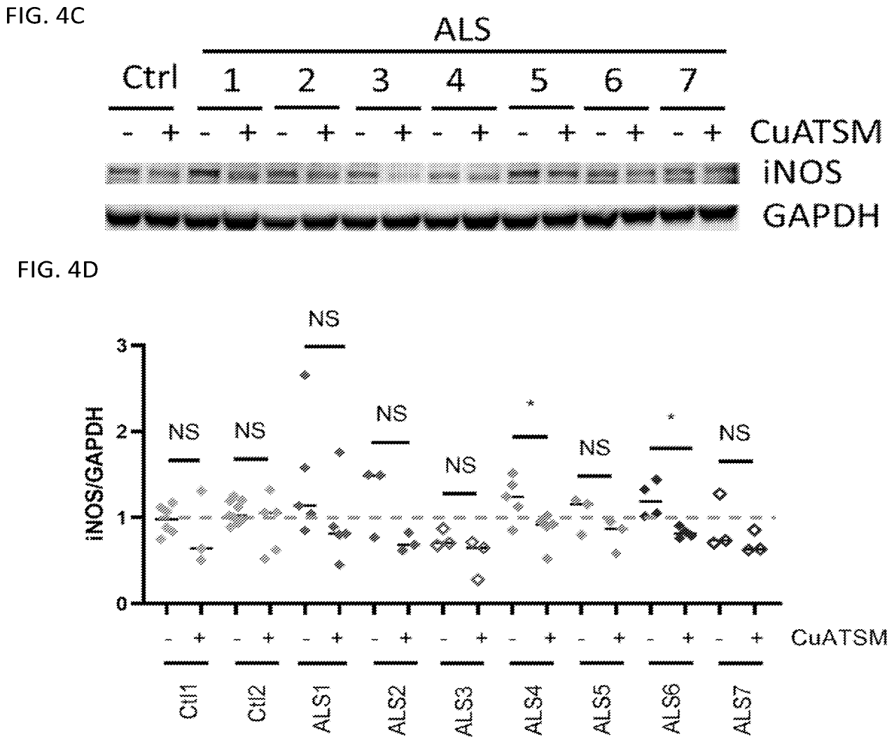

In addition, it was found that CuATSM impacted the levels of nitric oxide production (FIGS. 4A and 4B) in a patient specific manor. Both controls (Ctrl1 and Ctrl2), and all sALS (ALS1-3) and C9ORF72 (ALS6-7) patient iAs had elevation in nitric oxide levels, whereas the mutant SOD1 (ALS4-5) lines showed a reduction. Since nitric oxide is produced by inducible nitric oxide synthase (iNOS), the overall levels of iNOS was quantified by western blot. While CuATSM had no effect on nitric oxide synthase levels for most patient lines, there were two lines (ALS4 and ALS6) where these levels were reduced (FIGS. 4C and 4D). However, changes in iNOS levels do not directly correlate to changes in nitric oxide production for ALS6. While CuATSM resulted in some change in nitric oxide levels, these changes were not correlated to changes in iNOS protein levels following CuATSM treatment. Thus iNOS and nitric oxide production are not mediating the therapeutic effect of CuATSM. This further supports the argument that iNOS and nitric oxide does not play a role in CuATSM therapeutic effect. Combined this suggests that CuATSM effects in iAstrocytes were due primarily to modifying mitochondrial activity.

### Example 5

This example provides a discussion of the results obtained from the experiments of Examples 2-4.

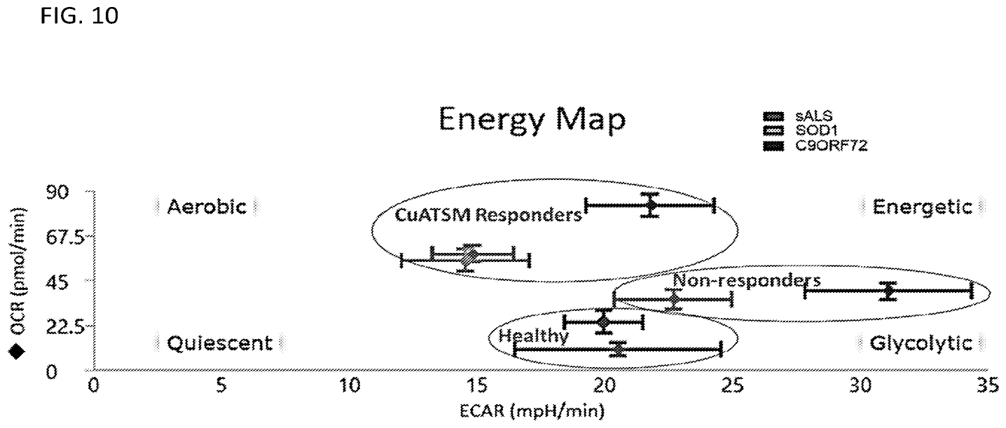

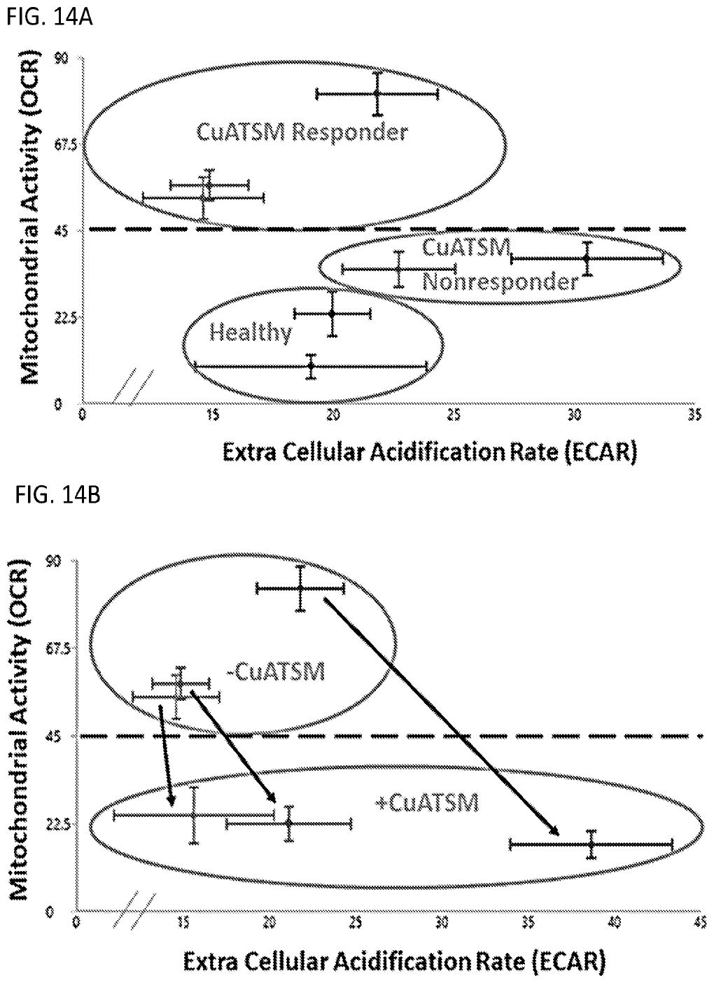

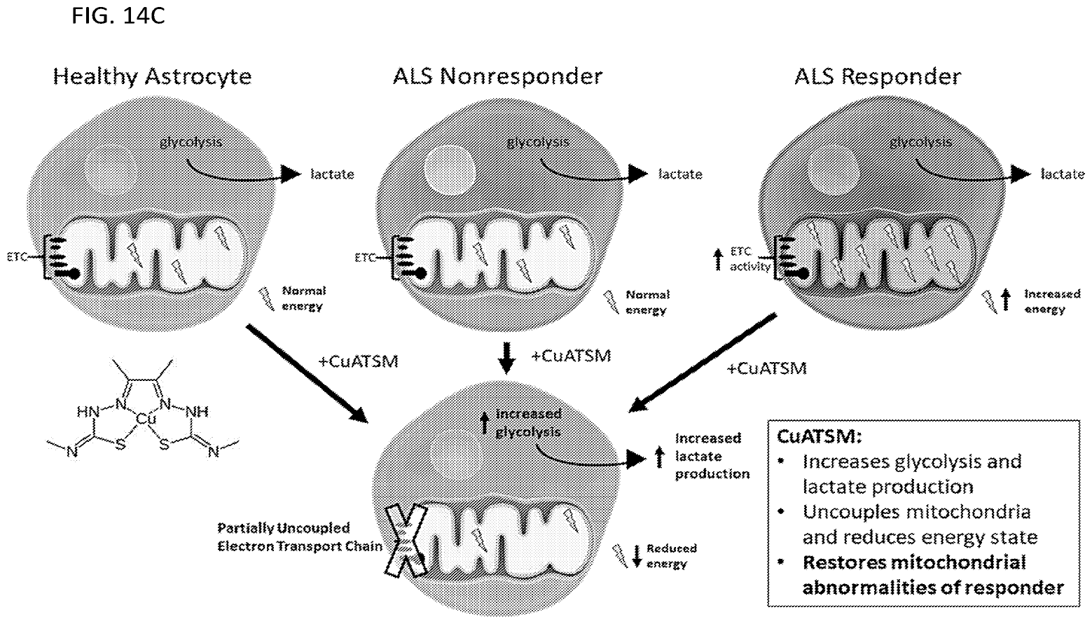

Here it was demonstrated that ALS patients that respond favorably to CuATSM treatment all have elevation in basal and/or ATP-linked respiration. In contrast, ALS patient nonresponders have basal and/or ATP-linked respiration at levels comparable or below healthy control levels (FIGS. 2C-2D and FIG. 7). It was proposed that the consequence of elevated ATP-linked respiration is elevated mitochondrial activity in these patient samples (FIG. 10 and FIG. 14A). Elevated mitochondrial activity may be an indicator of mitochondrial dysfunction or a biological response to elevated energy demands of the cell. Importantly, CuATSM treatment restores the mitochondrial activity of ALS responders to a healthy energy state (FIG. 14B).

Previous studies have shown that 1 μM CuATSM can degrade peroxynitrite in vitro preventing the formation of nitrotyrosine. Described herein is a novel therapeutic effect of CuATSM. Through the reduction of basal and/or ATP-linked respiration within the mitochondria (FIGS. 3A and 3B), CuATSM reduced the activity state of the mitochondria (FIG. 14B). Interestingly, despite the decrease in mitochondrial energy state following CuATSM treatment, there was an increase in superoxide production. While superoxide production can increase peroxynitrite formation resulting in oxidative stress, superoxide is also known to activate other intracellular pathways. The data suggests that following CuATSM treatment, there is either no change or a decrease in oxidative stress in the patient lines suggesting that the elevation in superoxide production is not causing oxidative stress (FIGS. 3D-3E). Thus, most likely, the elevation in superoxide production is activating protective signaling pathways within the astrocytes.

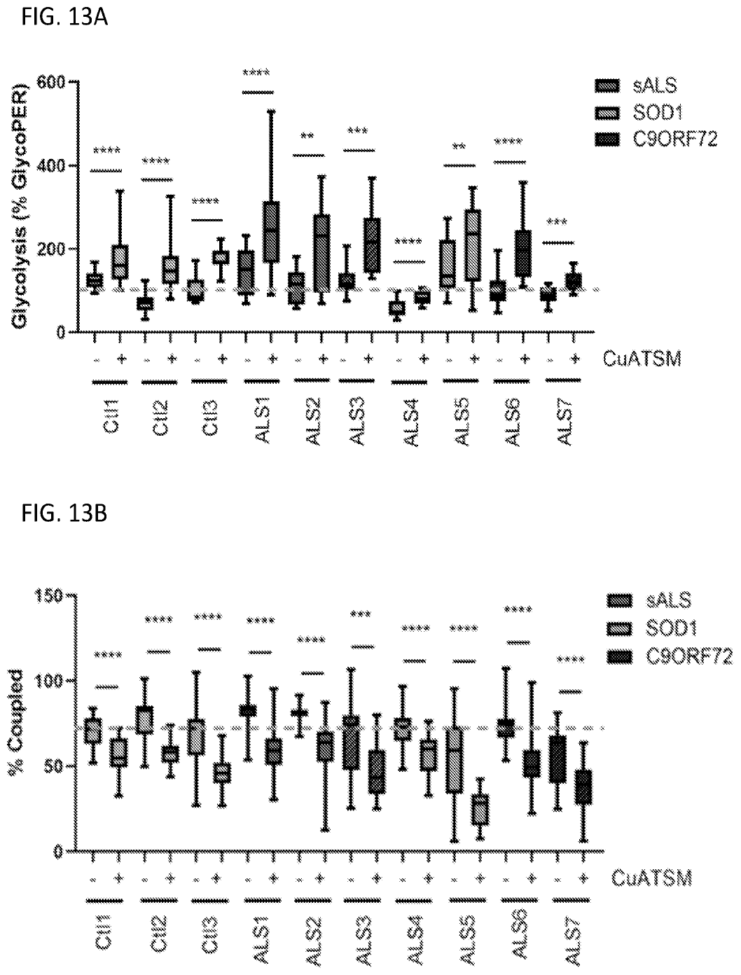

One potential mechanism is slight uncoupling of the mitochondria which can lead to increased superoxide production. In this application, we have shown that CuATSM leads to a slight decrease in mitochondrial uncoupling (FIG. 13B) as well as leads to an increase in superoxide production (FIGS. 3D and 3F).

Importantly, the data provided herein is the first to show in human iAstrocytes that CuATSM had a therapeutic effect on samples from sALS, SOD1 and C9ORF72 patients. To date, no other studies have described the therapeutic effect of CuATSM on C9ORF72 patients. The therapeutic effect of CuATSM on C9ORF72 patients is of particular interest as to date, these patient lines have been resistant to other therapeutic interventions attempted by our laboratory.

In addition, it is of particular interest that the SOD1 patient lines that were tested with CuATSM did not have mutations in the metal binding region of SOD1 protein. Thus the therapeutic effect of CuATSM is not limited to restoring the binding of copper to SOD1. This supports the notion that CuATSM has more than one therapeutic effect.

Finally, the in vitro model system described herein can be used to obtain preclinical efficacy data for neurological diseases that lack a mouse model. This system is useful for predicting patient responsiveness to a particular therapy. These applications may be useful for inclusion criteria and data interpretation of future clinical trials.

### Example 6

This example demonstrates patients with SCN2A mutations are candidates for CuATSM treatments.

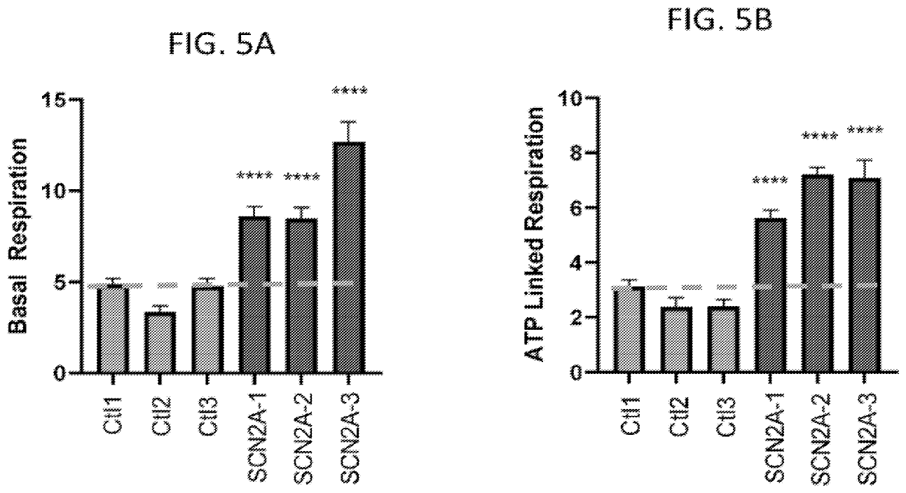

Preliminary observations made while characterizing SCN2A iAstrocytes suggest that mitochondrial dysfunction may play a role in the disease mechanism. Thus, the mitochondrial activity of iAstrocytes from SCN2A patients was evaluated, and these patients demonstrated an elevated basal respiration (FIG. 5A) and/or ATP-linked respiration (FIG. 5B). These findings suggest that SCN2A iAstrocytes have increased mitochondrial activity. Since metabolism is closely tied to these outcome measures, these findings also suggest that SCN2A iAstrocytes may have increased metabolic activity. The role of astrocytes in providing metabolic support for neurons and regulating neurotransmission suggests that these cells, in addition to neurons, may be a potential therapeutic target in patients with neurological disorders including SCN2A mutation-related disorders.

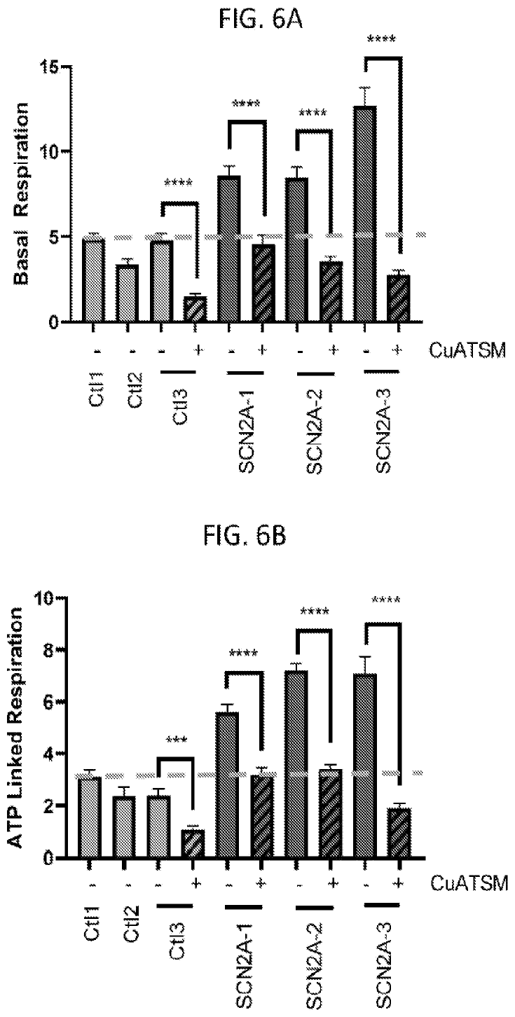

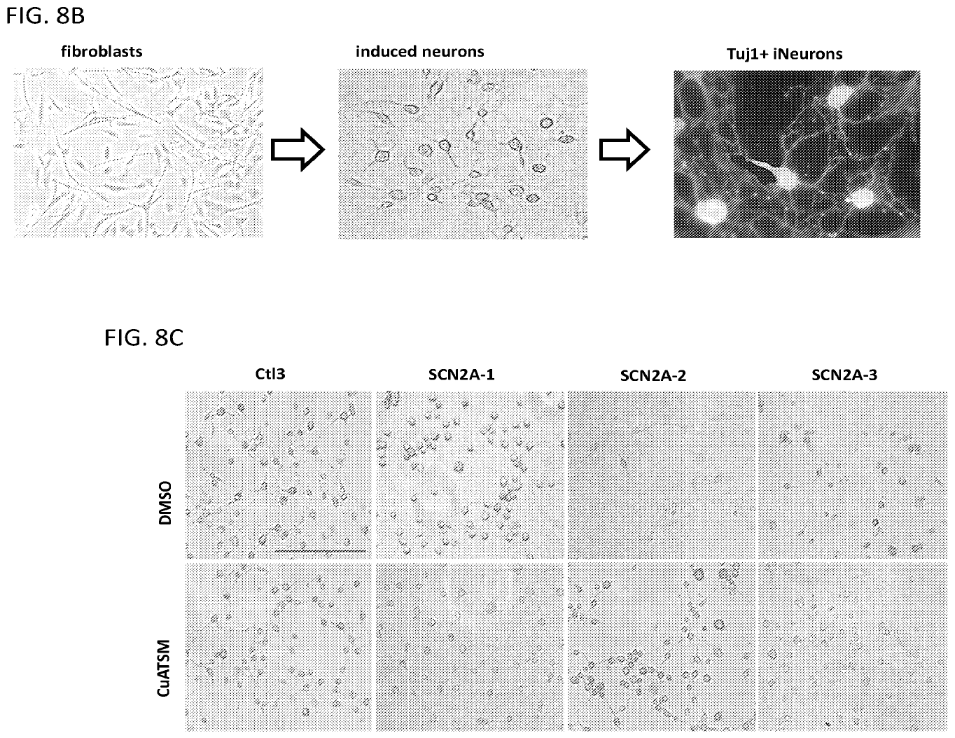

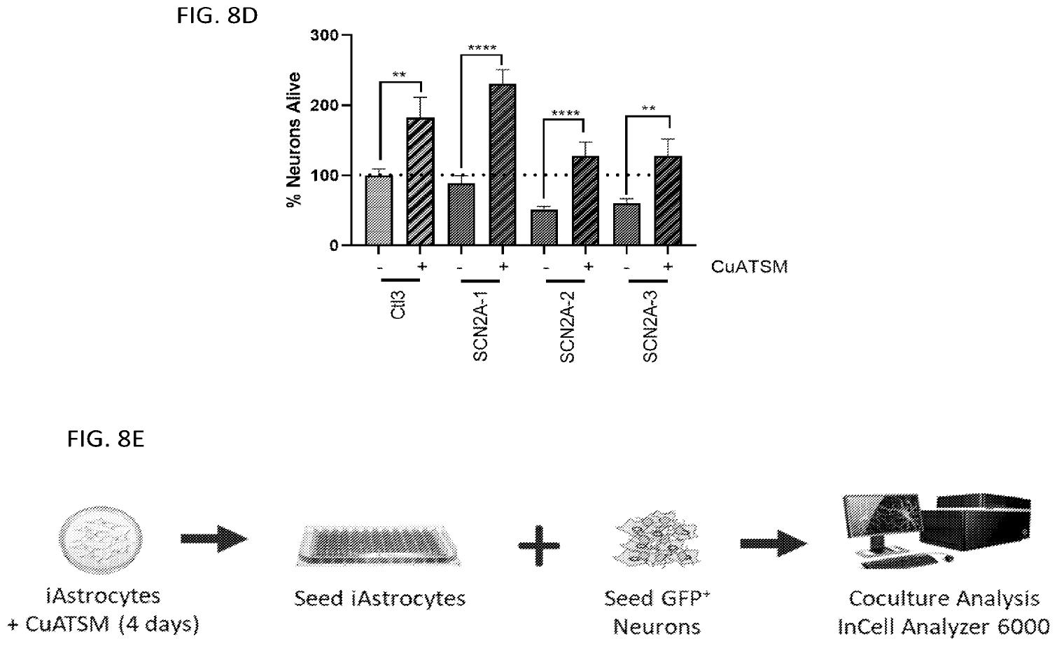

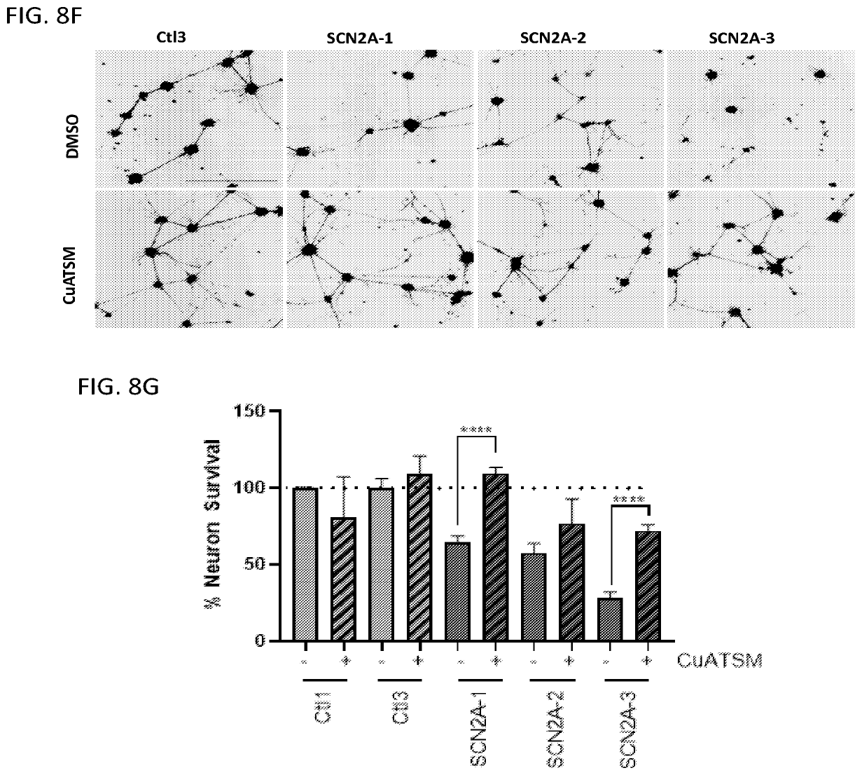

Given that elevation in mitochondrial activity is an indicator of patient responsiveness to CuATSM treatment, the effects of CuATSM on mitochondrial basal and ATP-linked respiration of SCN2A iAstrocytes was investigated. It was found that SCN2A iAstrocytes treated with CuATSM had reduced basal respiration (FIG. 6A) and ATP-linked respiration (FIG. 6B) in comparison to corresponding untreated controls. This data demonstrates that CuATSM is able to restore basal and ATP-linked respiration in SCN2A astrocytes to healthy levels and that SCN2A astrocytes are CuATSM responders. Finally, we looked at the effect of SCN2A mutations on neurons directly reprogramed from patient fibroblasts. It was observed that patient cells lines with SCN2A mutations had reduced number of neurons following differentiation (FIGS. 8C and 8D). Addition of CuATSM significantly improved the number of neurons following differentiation when compared to the corresponding untreated line (FIGS. 8C and 8D). In summary, the astrocyte and neuronal data presented suggests CuATSM to be a promising therapeutic for patients with SCN2A mutation-related disorders.

The effect of CuATSM treatment on SCN2A iAstrocytes mediated neuron toxicity was investigated. FIG. 8A shows a schematic protocol of induced neuron (iNeuron) reprogramming from patient fibroblasts. Fibroblast cells were seeded on 12-well plates and cultured in human fibroblast medium (HFM). Fibroblast cells were then differentiated for 7 days into iNeurons in the presence of induction medium and treatment a chemical cocktail VCRFSGY (valproic acid; CHIR99021; Repsox; SP600125 (JNK inhibitor), G06983 (PKC inhibitor) and Y-27632 (ROCK inhibitor)). To verify differentiation, the iNeurons were immunostained for the neuronal marker, Tuj1 (FIG. 8B). Representative bright field images of iNeurons following seven days of differentiation are shown in FIG. 8C. On days five and seven of differentiation, cells were treated with 0.01% of CuATSM. Neuron survival was quantified following seven days of differentiation by counting cells with neurites two times the soma length. There was reduce neuron survival in two SCN2A patient lines (SCN2A-2 and SCN2A-3) following differentiation (FIGS. 8C-8D). Importantly, CuATSM treatment was found to increase neuronal survival of SCN2A induced neurons (FIGS. 8C-8D). FIG. 8E shows a schematic of the drug screen co-culture assay performed. Astrocytes were pretreated with CuATSM or DMSO (vehicle control) during differentiation and seeded to a 96 well plate in the absence of CuATSM to form a monolayer. Twenty-four hours later, motor neurons were seeded on top of the astrocyte monolayer and viability was determined following 3 days in culture. FIG. 8F provides images of wild type GFP-neurons following 3 days in co-culture with patient SCN2A iAstrocyte cells. Quantification of these neurons show a reduction in neuronal survival when cultured in the presence of SCN2A mutated astrocytes (FIG. 8G). Quantification of neuron survival following co-culture indicates that CuATSM improved mouse neuron survival. Importantly, CuATSM pretreatment of patient iAstrocytes was able to significantly increase neuron survival of SCN2A-1 and SCN2A-3 with a trend towards increased survival in SCN2A-2 (FIG. 8G). In the case of SCN2A-2 more replications are likely need to show significance.

### Example 7

This example demonstrates patients with SCN2A mutations are candidates for CuATSM treatments.

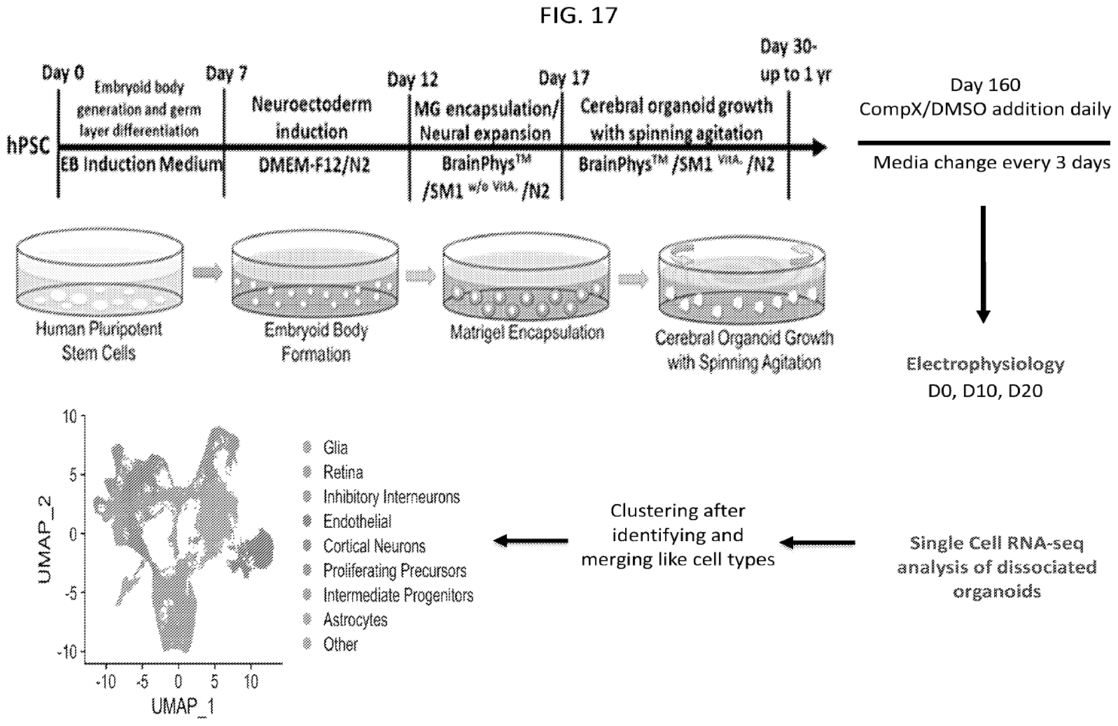

In addition, SCN2A brain organoids were generated to further investigate the effect of CuATSM treatment. SCN2A human pluripotent stem cells (hPSC) cells were converted into brain organoids as shown FIG. 17 and as follows. hPSC were induced to form embryoid bodies (EB) by use of EB induction media (day 0-6). Neuroectoderm induction was further promoted by switching from EB induction media to neural induction media (day 7-11). Media was switched to differentiation media and EBs were encapsulated in matrigel droplets to promote neural expansion (day 12-16). Differentiation media was continued to promote cerebral organoid growth with spinning agitation (day 17-30) as described in FIG. 17

On day 160, unrelated control and SCN2A (S4 patient) brain organoids were treated with DMSO/CuATSM (50 nM) daily, with media changes every three days, for up to 20 days. Electrophysiology readings were taken on day 0, day 10, and day 20 during the treatment period. Following the 20^{th} day of treatment, organoids were dissociated and single cell RNA-sequence analysis was performed using the 10× genomics platform. Cell clusters were defined using a hierarchical approach, first identifying neuronal vs. non-neuronal populations, then narrowing the definition based on specific markers, and finally merging clusters representing the same broad cell types. Clusters of distinct cell types were mapped as shown in FIG. 17. Uniform manifold approximation and projection (UMAP) for dimension reduction was used for visualization.

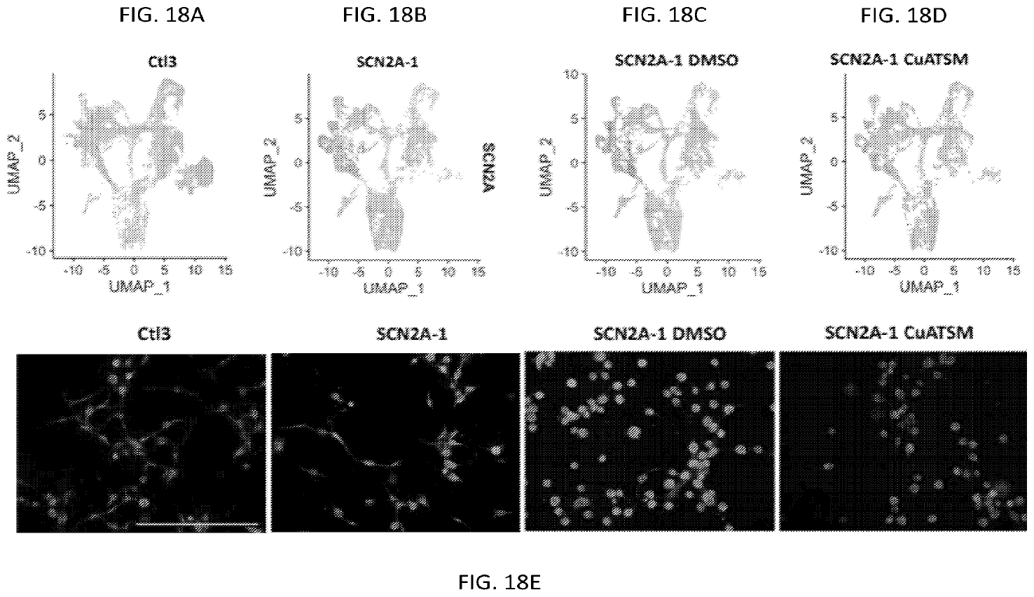

Next, SCN2A patient brain organoids were treated with CuATSM and RNA Seq analysis was performed. SCN2A patient (SCN2A-1) brain organoids were found to have an elevated expression of SCN2A gene versus DMSO treated or untreated controls (FIG. 18A-18C). Uniform manifold approximation and projection (UMAP) for dimension reduction was used for visualization. The expression of SCN2A was found to be higher within cortical and inhibitory interneuron cell clusters of patient brain organoids (FIGS. 18B and 18C) compared to control (FIG. 18A). Importantly, expression of SCN2A gene is reduced upon treatment with CuATSM (FIG. 18D). These findings were confirmed by directly differentiating the same patient fibroblasts (SCN2A-1) into induced neurons. Neurons were treated with CuATSM on day 5 and 7 of differentiating then stained for Nav1.2. Here we found that Nav1.2 protein levels were reduced following CuATSM treatment which confirms RNA seq data from organoids (FIG. 18E). Thus CuATSM can also downregulate SCN2A expression in diseased iAstrocytes which may add additional therapeutic benefit for these patients.

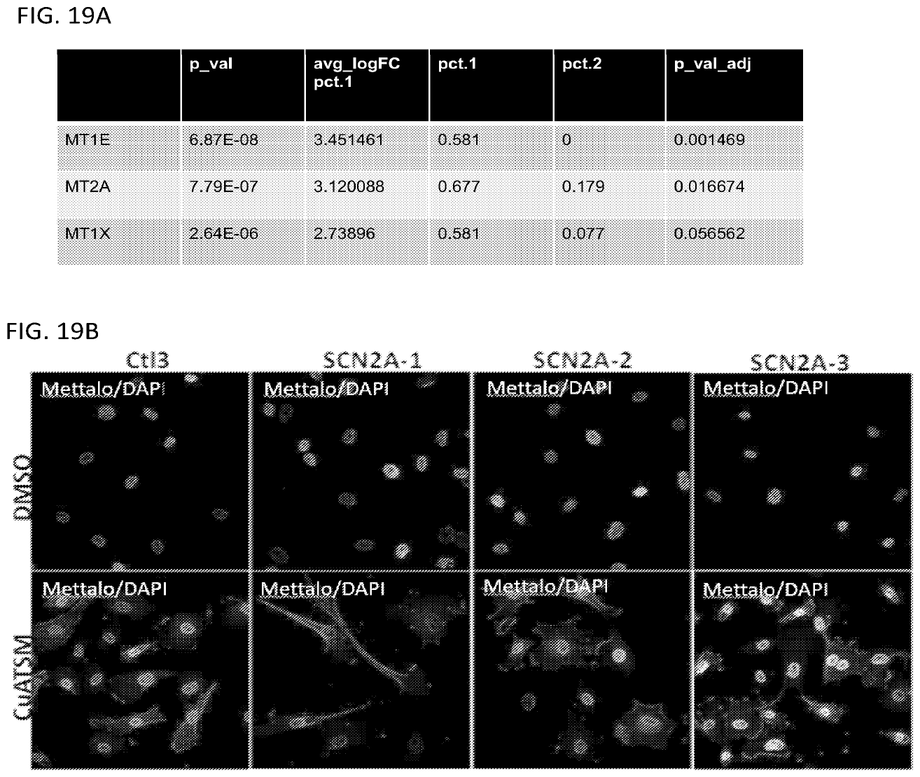

Metallothionine mRNA levels were found to be upregulated following CuATSM treatment. Single Cell RNA sequencing analysis of dissociated organoids was performed following treatment with CuATSM. Brain organoids from SCN2A (SCN2A-1) CuATSM responder were treated with DMSO control or CuATSM. CuATSM treated SCN2A brain organoids within the astrocytes cluster showed increase in metallothionine enzymes (MT1E, MT2A and MT1X) (FIG. 19A). To confirm these findings, SCN2A iAstrocytes were treated with CuATSM on days 2-5 during their differentiation in 10 cm plates. On day five, cells were seeded onto 24-well plates with cover slips. On day seven, cells were fixed with 4% paraformaldehyde (PFA) and immunostained for MT1 protein. MT1 was barely detectable in DMSO-treated cells, while CuATSM treatment caused an increase in MT1 expression (FIG. 19B). Together these findings show that CuATSM treatment upregulates metallothionine levels.

### Example 8

This example demonstrates that a rapid reprogramming method differentiates CuATSM responders/nonresponders from an ALS patient population.

Background: Patient diversity and unknown disease cause is a major challenge for drug development and clinical trial design. Heterogenicity in ALS patients (sALS and fALS) is not reflective in current animal models used to evaluate therapies and therefore, the direct translation of potential therapeutics using these models have proven difficult. Direct reprogramming facilitates compound screening on sALS and fALS lines and therefore the data indicate diverse patient response to therapeutic agent suggesting shared pathways between patient subgroups. In this study, one goal was to identify patient responders to CuATSM treatment and distinguish them from nonresponders. An evaluation of ALS disease markers identified increased mitochondrial activity as the shared parameter unique to responders.

Results: As shown in FIGS. 1B and 1C, CuATSM treatment of ALS astrocytes rescued motor neuron survival. As shown in FIGS. 2C-2E, ALS CuATSM responders had elevated basal and ATP-linked respiration in addition to increased complex IV (COX IV) activity. Striped lines indicate patient lines classified as nonresponders (ALS3 and ALS7) in co-culture assay. In addition, energy map plotting oxygen consumption (OCR) and lactate production (extracellular acidification rate, ECAR), using extracellular flux analysis (Seahorse) indicated CuATSM responders had elevated mitochondrial activity (FIG. 10). Here the data shows that the energy state of lines from patient CuATSM responders is dysregulated and the cells operate at a higher energetic level than both nonresponders and healthy controls. Importantly, CuATSM can reduced mitochondrial activity to healthy control levels (FIG. 3A-B and FIG. 14B). Here, seahorse analysis indicates that CuATSM reduces mitochondrial basal and/or ATP-linked respiration to levels at or below healthy controls. Striped lines indicate patient lines classified as nonresponders in co-culture assay (ALS3 and ALS6). Dotted lines indicate healthy mitochondrial activity maximum, determined based on co-culture of healthy astrocytes. This data supports our proposal that CuATSM responders have elevated mitochondrial activity that is restored to healthy levels following CuATSM treatment.

Conclusion: Diverse patient response to therapeutic compounds such as CuATSM suggests shared pathways are dysregulated between patient subgroups, e.g., CuATSM responders have mitochondria operating at a high energy state, and CuATSM can restore mitochondria to healthy activity levels. Patient iAstrocytes can be used to identify both disease modifiers and pathways dysregulated in a given individual potentially predicting therapeutic responsiveness, e.g., elevation in mitochondrial activity (basal and ATP linked respiration) may indicate patient is a strong candidate for CuATSM based therapies. An enhanced understanding of cellular profiles could aid clinicians in determining best treatment approach for patients.

### Example 9

This example investigates patients with SLC6A1 mutations as candidates for CuATSM treatments.

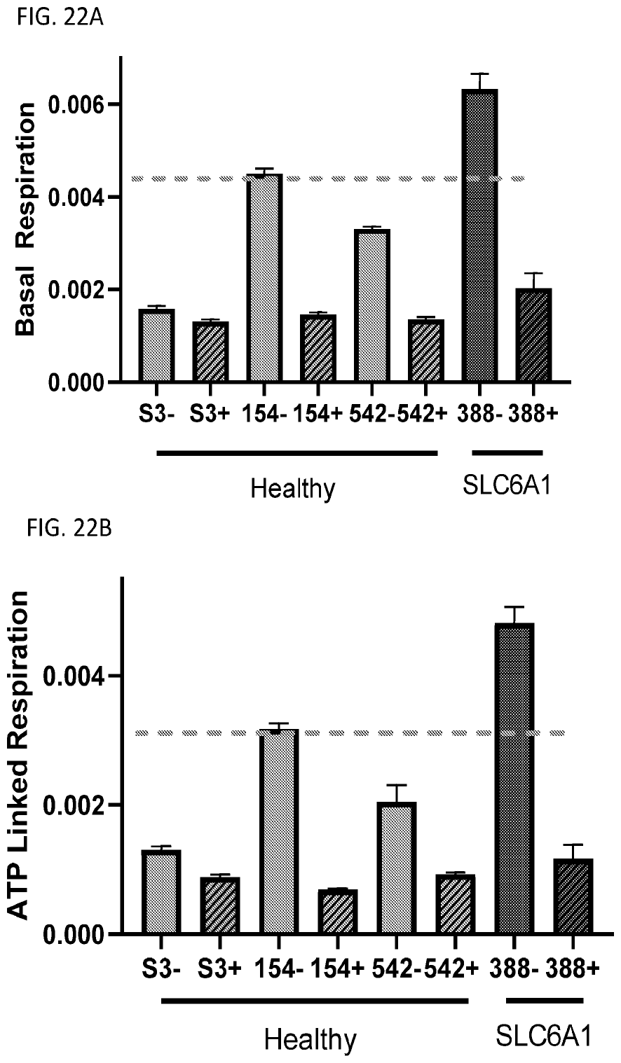

The role of astrocytes in providing metabolic support for neurons and regulating neurotransmission suggests that these cells, in addition to neurons, may be a potential therapeutic target for patients with neurological disorders including SLC6A1 mutations. The effects of CuATSM on mitochondrial activity of iAstrocytes from SLC6A1 mutation patients was investigated. Basal respiration and ATP-linked respiration was measured in SLC6A1 iAstrocytes and compared to healthy controls. In addition, SLC6A1 iAstrocytes were also treated with CuATSM (+) and compared to corresponding untreated controls. This study demonstrated that SLC6A1 patient iAstrocyte had elevated basal and ATP-linked respiration (FIGS. 22A and 22B). CuATSM treatment reduced both basal and ATP-linked respiration to levels below the controls (FIGS. 22A and 22B). Finally, since SLC6A1 mutations also impact neurons, the effect of CuATSM treatment on neuronal differentiation is also investigated.

### Example 10

This example investigates CuATSM treatments of mouse knockin model of early onset absence epilepsy harboring a SLC6A1 mutation.

A mouse model of early onset absence epilepsy harboring a A288V mutation of the SLC6A1 gene is utilized to assess CuATSM treatment. The SLC6A1^{+/A288V} mice were generated using CRISPR/Cas9 global knockin with a FLeX targeting vector. In this model the SLC6A1 mutation can be activated in a spatiotemporal (specific time and cell type) by breeding with tissue specific CreERT2 mice.

Neurons and astrocytes derived from the SLC6A1^{+/A288V} mice are evaluated following treatment with CuATSM or corresponding untreated controls. Levels of basal cellular respiration, mitochondrial ATP-linked respiration are measured in CuATSM-treated and corresponding untreated controls. The effect of CuATSM treatment on neuronal differentiation is also investigated.

### Example 11

This example investigates ALS markers in iAstrocytes following CuATSM treatment.

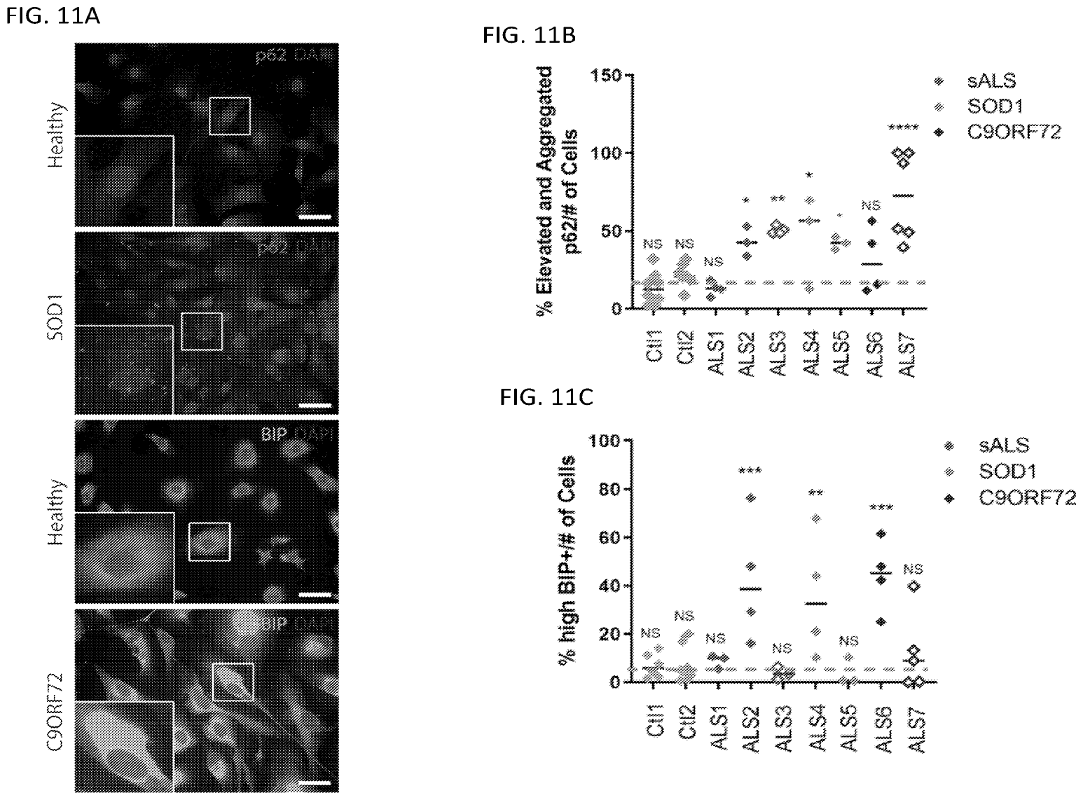

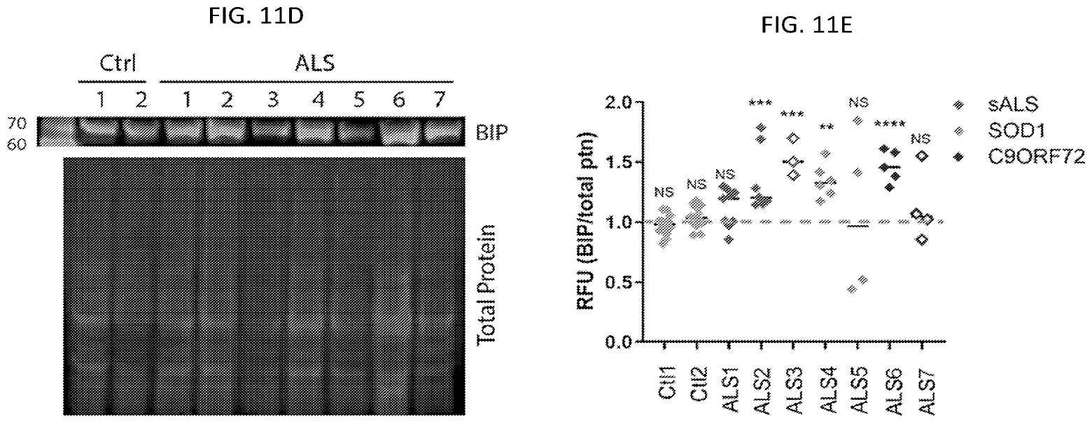

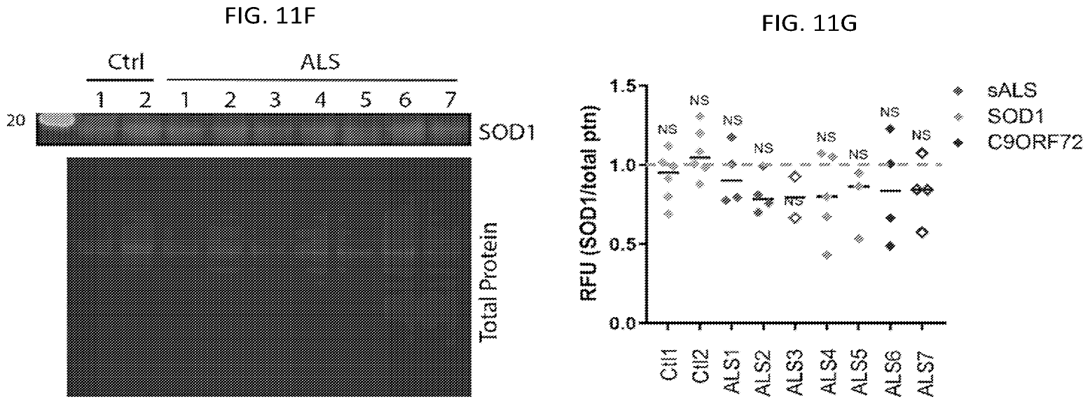

Preliminary characterizations of protein aggregation, endoplasmic reticulum (ER) and oxidative stress of the patient samples support this observation as iAstrocytes cellular profile are highly variable between individuals. ALS markers (p62 and BIP) were compared between CuATSM responder and nonresponder iAstrocytes to identify pathways that could distinguish these two groups. While immunostaining and blind quantitation of ALS iAstrocytes indicated increased levels and aggregation of p62 in CuATSM responders (ALS2, ALS4, and ALS5) and nonresponders, (ALS3 and ALS7) compared to healthy controls, additional iAstrocytes responders (ALS1 and ALS6) did not show significant changes (FIGS. 11A-11B). These results suggest that elevation and aggregation of p62 does not distinguish responders from nonresponders.

Activation of the ER stress response was then investigated by immunostaining for BIP. As previously observed for p62, BIP immunostainings also showed differential protein levels between individual cells of a given patient line (FIG. 11A). Automated quantification of the number of cells with elevated BIP showed significant increase in CuATSM responders (ASL2, ALS4 and ALS6) in comparison to healthy controls (FIG. 11C). In contrast, the number of cells with elevated BIP in both responders (ALS1 and ALS5) and nonresponders (ALS3 and ALS7) were not significantly different to those of healthy controls (FIG. 11C). In addition, the total BIP levels were also determined in the different lines by western blot (FIGS. 11D-11E). CuATSM responders (ALS2, ALS4, and ALS6) and ALS3, a nonresponder, all had significant elevation of BIP in comparison to healthy controls (FIG. 11E). In contrast, responders (ALS1 and ALS5) and ALS7, a nonresponder did not show BIP elevation (FIG. 11E). Combined, this data suggests that ER stress is not a predictor of CuATSM responsiveness in the ALS iAstrocytes.

It has been shown that CuATSM can also function as an scavenger of the oxidant peroxynitrite (Hung et al, JEM, 2012). It was then investigated whether SOD1 levels could distinguish responders from nonresponders by influencing the levels of oxidative stress. Western blot analysis of SOD1 levels for all patient iAstrocytes indicated no significant difference in SOD1 levels between ALS iAstrocytes and healthy controls (FIGS. 11F-11G). These data suggest that p62 aggregation, ER stress and SOD1 levels do not distinguish CuATSM responders from non-responders.

### Example 12

This example investigates mitochondrial respiration of iAstrocytes in response to CuATSM treatment.

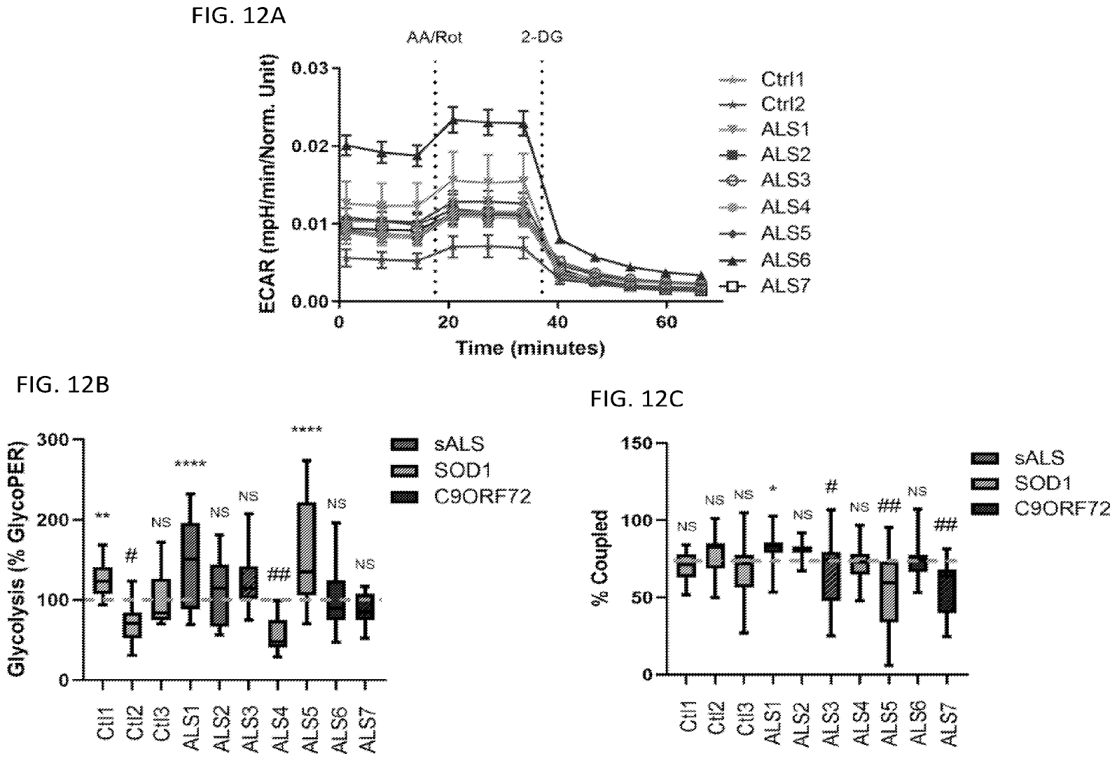

While variation in ALS disease markers is common amongst patients and animal models, one of the most shared abnormalities is dysregulated energy metabolism (Dupuis et al., Lancet Neurol. 2011; 10(1):75-82). Given that astrocyte are primarily glycolytic, cellular glycolysis in patient iAstrocytes were measured by extracellular flux analysis and a representative rate graph is shown (FIG. 12A). Only ALS1 and ALS5 had significantly elevated glycolysis where as ALS4 had a significant reduction when compared to healthy controls (FIGS. 12A and 12B).

Given that CuATSM treatment significantly reduced the mitochondrial energy state of responders (FIG. 14B), we further proposed that reduction in mitochondrial coupling may distinguish responders from nonresponders. While most untreated patient lines showed similar levels of coupled mitochondria, one ALS responder (ALS5) and both ALS nonresponder (ALS3 and ALS7) had a significant reduction in coupled mitochondria compared to healthy controls. In addition, ALS1, a responder, had a small but significant increase in mitochondria coupling respect to controls (FIG. 12C). Thus, no link was found between mitochondrial coupling (determined as the percentage of basal respiration linked to ATP production) and patient line responsiveness to CuATSM.

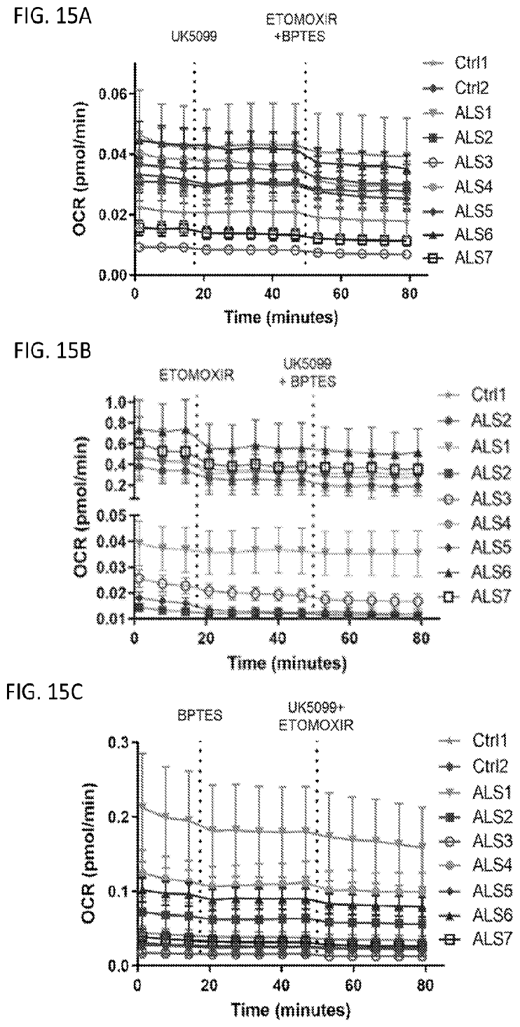

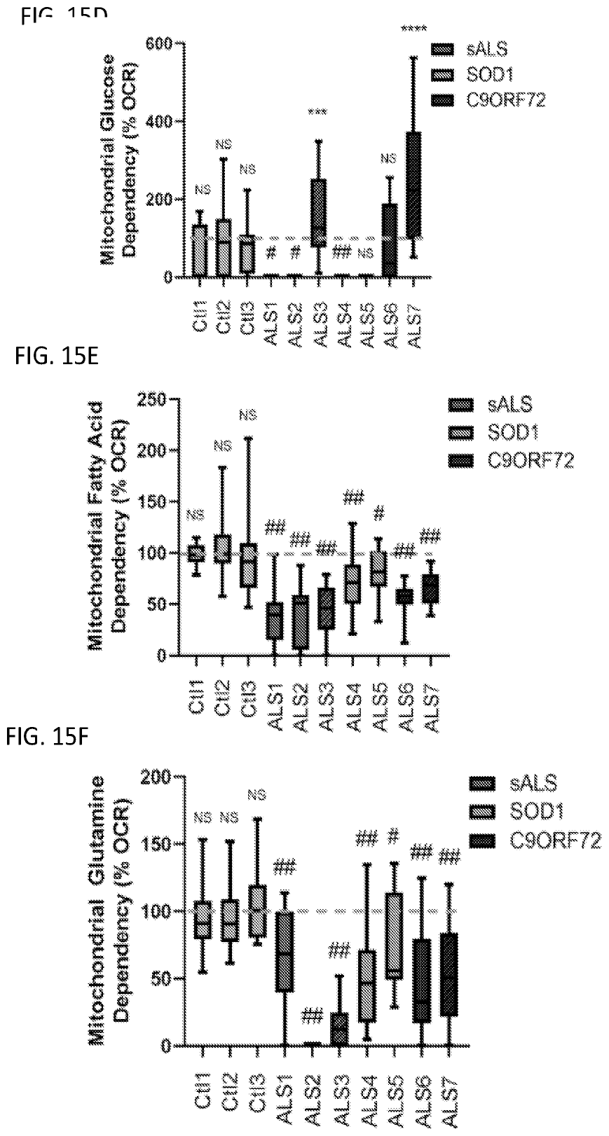

Changes in mitochondrial dependency on different fuel sources may explain the variation in oxidative phosphorylation observed between patient lines. Thus, mitochondrial dependency on glucose, glutamine and long chain fatty acids as fuel sources was examined by extracellular flux analysis and representative rate graphs are shown (FIGS. 15A-C). A significant decrease in the dependency of all ALS responders and nonresponders for glutamine and fatty acids was observed (FIGS. 15E and 15F). In addition, it was found that all ALS CuATSM responders (ALS1, ALS2, ALS4, ALS5 and ALS6) were not dependent on glucose for mitochondrial energy production. In contrast nonresponders (ALS3 and ALS7) had mitochondria that were more dependent on glucose than healthy controls (FIG. 15D). While these results suggested that the patient lines seem to have more flexibility in the use of different fuel sources than healthy controls, it appears that CuATSM nonresponders may preferentially utilize glucose.

### Example 13

This example investigates CuATSM treatment on mitochondrial activity.

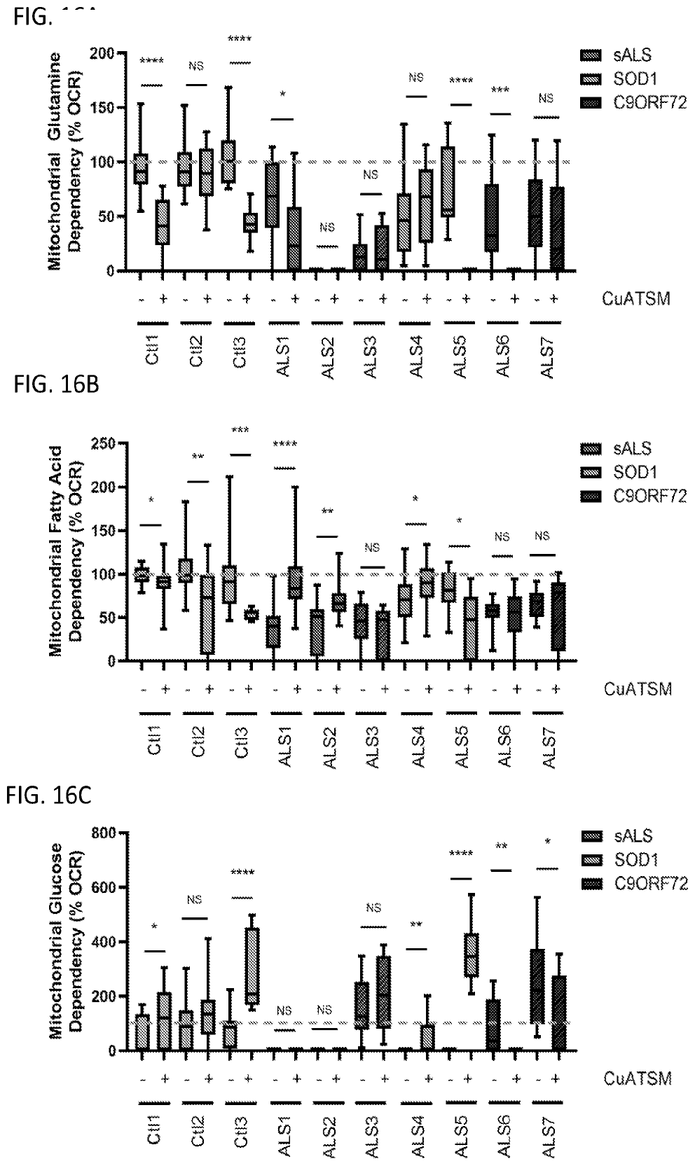

Astrocytes provide critical metabolic support for motor neurons by releasing lactate that can be used as a source of energy, thus glycolysis is a key metabolic phenotype of astrocytes. It is possible that the observed effect of astrocytes incubated with CuATSM on motor neuron survival could be due to an increase in glycolysis and release of lactate to the cell culture medium. A significant increase in cellular glycolysis was observed following CuATSM treatment on every cell line tested, including responders, nonresponders and healthy controls (FIG. 13A). Next, given that CuATSM responders showed elevated mitochondrial respiration, that is subsequently reduced with CuATSM (FIGS. 3A and 3B) we sought to determine if this reduction was caused due to changes in mitochondrial fuel dependency. However, mitochondrial dependency analysis following CuATSM treatment indicated that while CuATSM can mediate changes in the mitochondrial dependency on certain substrates, the impact on fuel sources is a patient specific response. Thus, this reduction in activity was not due to a decrease in the mitochondrial dependency on glutamine, fatty acid or glucose following CuATSM addition (FIGS. 16A-16C). Importantly, mitochondrial dependency on glucose was not changed between CuATSM nonresponders indicating that the nonresponders increased dependency on glucose was separate to CuATSM's therapeutic effect. Based on these observations it was next determined whether CuATSM was impacting mitochondrial coupling. A modest, yet significant reduction in coupling for all iAstrocytes treated with CuATSM was observed (FIG. 13B). This slight uncoupling may explain why the energy state of the mitochondria is reduced following CuATSM treatment. Importantly, since the mitochondria of ALS nonresponders already are at a lower energy state, the impact of CuATSM mediated uncoupling has no therapeutic effect. Thus, CuATSM may improve iAstrocytes support of neurons through elevation of glycolysis and subsequent lactate production in addition to reducing mitochondrial activity through reduction in mitochondrial coupling. Further, elevation in mitochondrial activity is a common feature only to responders and this feature is corrected following CuATSM treatment. Thus mitochondrial activity could be an effective and selective marker to predict patients' responsiveness to CUATSM treatment.

Combined, these metabolic data suggest that patient responders have a unique mitochondrial phenotype that distinguishes them from both healthy and ALS nonresponders. In fact, energy maps indicate that CuATSM responding lines have mitochondria that are at a higher energy state than healthy and nonresponding lines (FIGS. 10 and 14A). These findings suggest that elevation in mitochondrial activity may distinguish patient CuATSM responders from nonresponders. Importantly, the energy map indicated that CuATSM treatment of patient responders lowered their energy state to levels comparable to those of control lines (FIG. 14B). Thus, it was proposed that the mechanism of CuATSM action on ALS responders is due to mitochondrial uncoupling that lowers patient mitochondrial energy state. The lowered energy state in combination with the increase in glycolysis and subsequent lactate production lead to increased iAstrocyte mediated motor neuron protection (FIG. 14C).

### Example 14

This example investigates CuATSM treatment on IRF2BPL iAstrocytes.

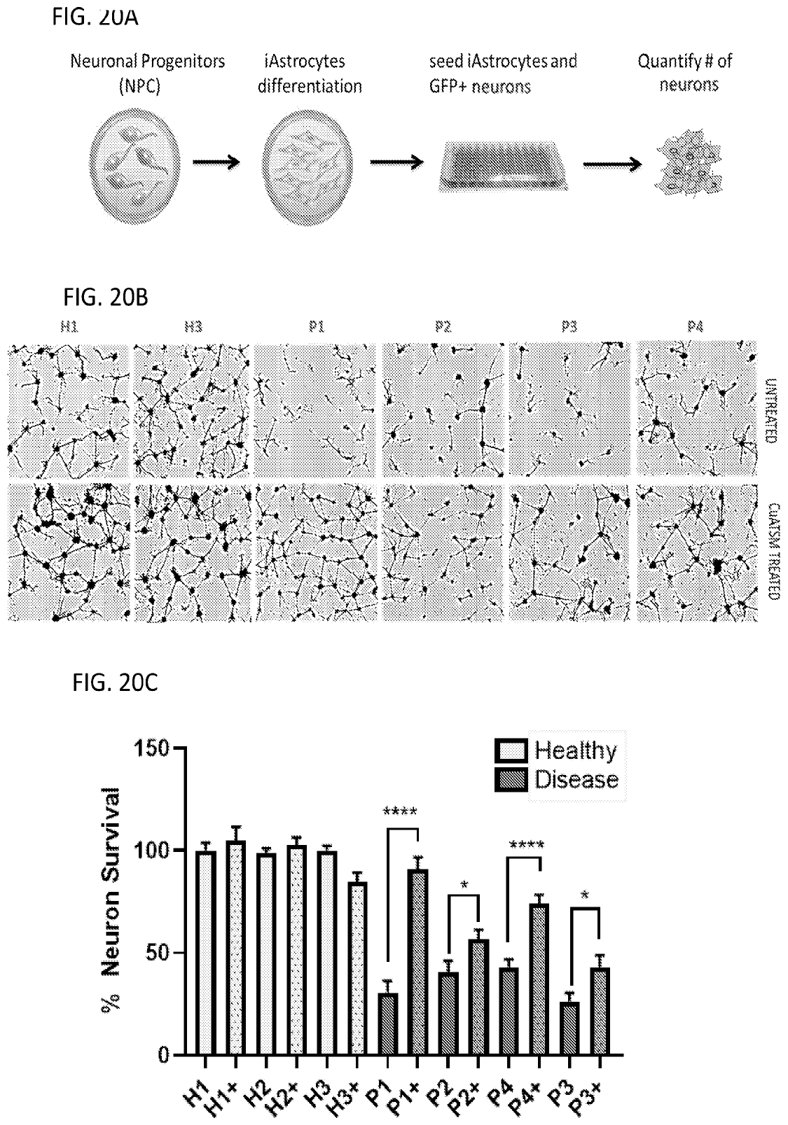

Astrocytes were subjected to a drug screen co-culture assay as shown in FIG. 20A. Astrocytes were treated with CuATSM starting day 2 of differentiation and then seeded in a 96 well plate in the absence of CuATSM to form a monolayer. Twenty hours later neurons were seeded on top of astrocyte monolayer and viability was determined following 3 days in culture (FIG. 20B). Motor neuron survival following co-culture was determined by quantifying the number of surviving neurons. These data showed that IRF2BPL astrocyte were toxic to neurons and that CuATSM treatment of IRF2BPL prevented this toxicity (FIG. 20C). Thus, this data would suggest that IRF2BPL patient iAstrocytes will respond therapeutically to CuATSM.

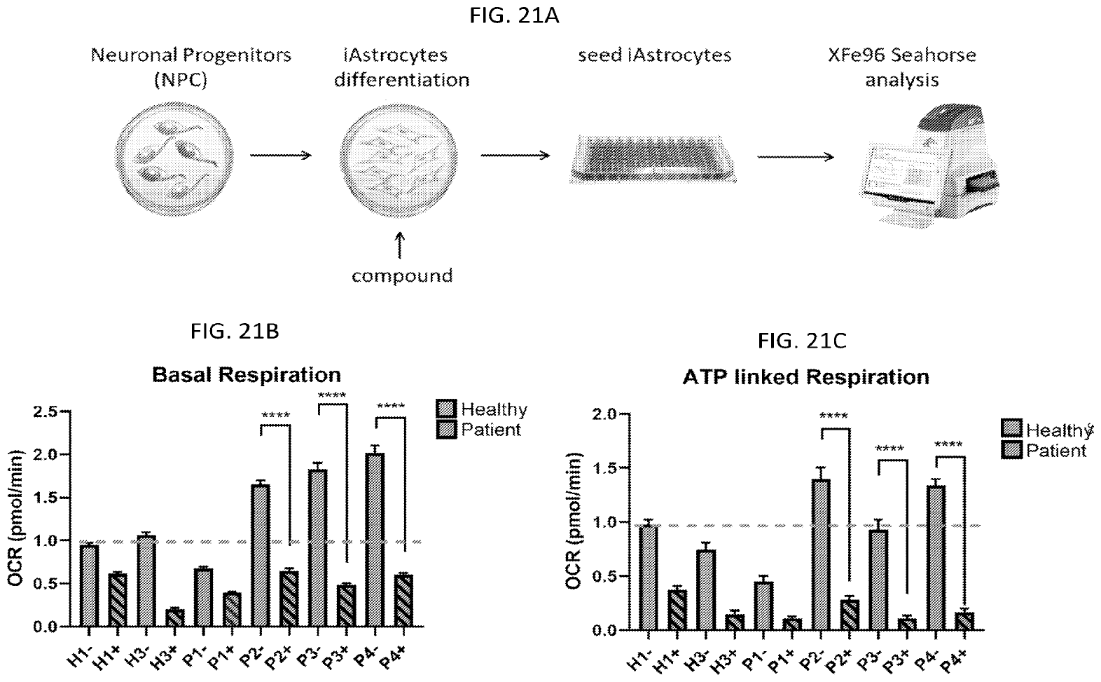

Next, the effect of CuATSM treatment on basal and/or ATP-linked respiration in NEDAMSS astrocytes was investigated. FIG. 21A shows a schematic image of the seahorse assay used. The base oxygen consumption rate (FIG. 21B) was measured at three time points for healthy and NEDAMSS patients astrocytes either treated or not treated with CuATSM. ATP linked respiration was measured by subtracting the OCR for basal respiration from the OCR following oligomycin (FIG. 21C). Three out of the four patient lines showed increased basal respiration and with two of four patients showing increased ATP-linked respiration (FIGS. 21B and 21C). For this disease, one of the patient cell lines tested (P1) that did not have an abnormal mitochondrial phenotype, but still showed therapeutic responsiveness in co-culture. This beneficial response may be due to a mutation specific alteration in one of the other pathways described herein that cuATSM affects that results in another abnormal pathway targeted by CuATSM. Importantly, CuATSM treatment was shown to reduce the elevated basal and/or ATP-linked respiration in all IRF2BPL (NEDAMSS) astrocytes.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted.

Recitation of ranges of values herein are merely intended to serve as a shorthand method of referring individually to each separate value falling within the range and each endpoint, unless otherwise indicated herein, and each separate value and endpoint is incorporated into the specification as if it were individually recited herein.

All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

Preferred embodiments of this disclosure are described herein, including the best mode known to the inventors for carrying out the disclosure. Variations of those preferred embodiments may become apparent to those of ordinary skill in the art upon reading the foregoing description. The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

What is claimed is:

1. A method of treating a subject comprising a SCN2A mutation, a mutated SCN2A voltage-gated sodium channel protein a SCN1A or an IRF2BPL mutation or a SLC6A1 mutation, comprising administering to the subject copper-ATSM (CuATSM) in an amount effective to treat the subject.

2. A method of treating a subject with elevated levels of basal mitochondrial respiration, mitochondrial ATP-linked respiration, or a combination thereof, comprising administering to the subject copper-ATSM (CuATSM) in an amount effective to treat the subject.

3. A method of treating a subject with a neurodegenerative disorder associated with mitochondrial dysfunction, or a neurodegenerative or neurological disorder associated with elevated levels of mitochondrial basal and/or ATP-linked respiration, comprising administering to the subject copper-ATSM (CuATSM) in an amount effective to treat the neurodegenerative disorder.

4. A method of treating a subject with a seizure disorder comprising administering to the subject copper-ATSM (CuATSM) in an amount effective to treat the subject.

5. A method of improving survival of motor neurons or other neuronal cell types, reducing mitochondrial basal and/or ATP-linked respiration, reducing cellular oxidative stress, or a combination thereof, in a subject, comprising administering to the subject copper-ATSM (CuATSM) in an amount effective to improve survival of motor neurons, reduce mitochondrial ATP-linked respiration, and/or reduce cellular oxidative stress in the subject.

6. The method of any one of claims 1-5, wherein CuATSM is administered to the subject once daily.

7. The method of any one of claims 1-6, wherein CuATSM is administered to the subject orally or intravenously.

8. The method of any one of claims 1-6, wherein CuATSM is administered to the subject via the cerebrospinal fluid (CSF).

9. The method of any one of claims 1-8, wherein CuATSM is administered in an amount effective to reduce the levels of basal and/or ATP-linked respiration in the induced astrocytes or neurons made from patient skin cells of the subject to a level that is equal to or less than a control level.

10. The method of any one of claims 1-8, wherein CuATSM is administered in an amount effective to reduce the levels of basal mitochondrial respiration in cells of the subject to a level that is equal to or less than a control level.

11. The method of claim 9 or 10, wherein the control level is a level of mitochondrial basal and/or ATP-linked respiration of a healthy, undiseased subject.

12. The method of any one of claims 1-11, wherein CuATSM is administered at a dosage of at least or about 1 mg/day.

13. The method of claim 12, wherein the dosage is at least or about 3 mg/day, at least or about 6 mg/day, at least or about 12 mg/day, at least or about 24 mg/day, at least or about 48 mg/day, at least or about 72 mg/day, or at least or about 100 mg/day.

14. A composition for treating a subject comprising a SCN2A mutation, a mutated SCN2A voltage-gated sodium channel protein a SCN1A or an IRF2BPL mutation or a SLC6A1 mutation, wherein the composition comprises copper-ATSM (CuATSM) in an amount effective to treat the subject.

15. A composition for treating a subject with elevated levels of basal mitochondrial respiration, mitochondrial ATP-linked respiration, or a combination thereof, wherein the composition comprises copper-ATSM (CuATSM) in an amount effective to treat the subject.

16. A composition for treating a subject with a neurodegenerative disorder associated with mitochondrial dysfunction or a neurodegenerative or neurological disorder associated with elevated levels of mitochondrial basal and/or ATP-linked respiration, wherein the composition comprises copper-ATSM (CuATSM) in an amount effective to treat the neurodegenerative disorder.

17. A composition for treating a subject with a seizure disorder, wherein the composition comprises copper-ATSM (CuATSM) in an amount effective to treat the subject.

18. A composition for improving survival of motor neurons or other neuronal cell types, reducing mitochondrial basal and/or ATP-linked respiration, reducing cellular oxidative stress, or a combination thereof, in a subject, the composition comprising copper-ATSM (CuATSM) in an amount effective to improve survival of motor neurons, reduce mitochondrial basal and/or ATP-linked respiration, and/or reduce cellular oxidative stress in the subject.

19. The composition of any one of claims 14-18, wherein the composition is formulated for administration to the subject once daily.

20. The composition of any one of claims 14-19, wherein the composition is formulated for administration to the subject orally or intravenously.

21. The composition of any one of claims 14-19, wherein the composition is formulated for administration to the subject via the cerebrospinal fluid (CSF).

22. The composition of any one of claims 14-21, wherein CuATSM is in an amount effective to reduce the levels of basal and/or ATP-linked respiration in the astrocytes of the subject to a level that is equal to or less than a control level.

23. The composition of any one of claims 14-22, wherein CuATSM is in an amount effective to reduce the levels of basal mitochondrial respiration in cells of the subject to a level that is equal to or less than a control level.

24. The composition of claim 22 or 23, wherein the control level is a level of mitochondrial ATP-linked respiration of a healthy, undiseased subject.

25. The composition of any one of claims 14-24, wherein CuATSM is at a dosage of at least or about 1 mg/day.

26. The composition of claim 25, wherein the dosage is at least or about 3 mg/day, at least or about 6 mg/day, at least or about 12 mg/day, at least or about 24 mg/day, at least or about 48 mg/day, at least or about 72 mg/day, or at least or about 100 mg/day.

27. Use of a copper-ATSM (CuATSM) for the preparation of a medicament for treating a subject comprising a SCN2A mutation, a mutated SCN2A voltage-gated sodium channel protein a SCN1A or an IRF2BPL mutation or a SLC6A1 mutation, wherein the medicament comprises copper-ATSM (CuATSM) in an amount effective to treat the subject.

28. Use of a copper-ATSM (CuATSM) for the preparation of a medicament for treating a subject with elevated levels of basal mitochondrial respiration, mitochondrial ATP-linked respiration, or a combination thereof, wherein the composition comprises copper-ATSM (CuATSM) in an amount effective to treat the subject.

29. Use of a copper-ATSM (CuATSM) for the preparation of a medicament for treating a neurodegenerative disorder associated with mitochondrial dysfunction or, a neurodegenerative or neurological disorder associated with elevated levels of basal and/or mitochondrial ATP-linked respiration, wherein the composition comprises copper-ATSM (CuATSM) in an amount effective to treat the neurodegenerative disorder.

30. Use of a copper-ATSM (CuATSM) for the preparation of a medicament for treating a seizure disorder in a subject in need thereof, wherein the medicament comprises copper-ATSM (CuATSM) in an amount effective to treat the subject.

31. Use of a copper-ATSM (CuATSM) for the preparation of a medicament for improving survival of motor neurons or other neuronal cell types, reducing mitochondrial basal and/or ATP-linked respiration, reducing cellular oxidative stress, or a combination thereof, in a subject in need thereof, wherein the medicament comprises copper-ATSM (CuATSM) in an amount effective to improve survival of motor neurons, reduce mitochondrial basal and/or ATP-linked respiration, and/or reduce cellular oxidative stress in the subject.

32. The use of any one of claims 27-31, wherein the medicament is formulated to be administered to the subject once daily.

33. The use of any one of claims 27-32, wherein the medicament is formulated to be administered to the subject orally or intravenously.

34. The use of any one of claims 27-31, wherein the medicament is formulated to be administered to the subject via the cerebrospinal fluid (CSF).

35. The use of any one of claims 27-34, wherein CuATSM is in an amount effective to reduce the levels of basal and/or ATP-linked respiration in the astrocytes of the subject to a level that is equal to or less than a control level.

36. The use of any one of claims 27-35, wherein CuATSM is in an amount effective to reduce the levels of basal mitochondrial respiration in cells of the subject to a level that is equal to or less than a control level.

37. The use of claim 35 or 36, wherein the control level is a level of mitochondrial basal and/or ATP-linked respiration of a healthy, undiseased subject.

38. The use of any one of claims 27-37, wherein CuATSM is at a dosage of at least or about 1 mg/day.

39. The use of claim 38, wherein the dosage is at least or about 3 mg/day, at least or about 6 mg/day, at least or about 12 mg/day, at least or about 24 mg/day, at least or about 48 mg/day, at least or about 72 mg/day, or at least or about 100 mg/day.

40. The method, composition or use of any one of claims 1-39, wherein the subject comprises a SCN2A mutation, a mutated SCN2A voltage-gated sodium channel protein a SLC6A1 mutation, a SCN1A mutation or a mutation in IRF2BPL.

41. The method, composition or use of any one of claims 1-40, wherein the subject comprises skin cells that can be reprogrammed into induced neuronal progenitor cells (iNPCs) that differentiate into iAstrocytes and/or neurons and/or oligodendrocytes which exhibit elevated levels of basal mitochondrial respiration, mitochondrial ATP-linked respiration, or a combination thereof.

42. The method, composition or use of any one of claims 1-41, wherein skin cells from the subject can be reprogrammed into induced neuronal progenitor cells (iNPCs) that differentiate into astrocytes, wherein the astrocytes exhibit an increased energy state.

43. The method, composition or use of claim 42, wherein the increased energy state is reflected by the increased oxygen consumption and increased lactate production or increased extracellular acidification rate, or a combination thereof of the astrocytes.

44. The method, composition or use of any one of claims 1-43, wherein the subject has a neurodegenerative or neurological disorder associated with mitochondrial dysfunction, optionally, a neurodegenerative disorder associated with elevated levels of mitochondrial basal and/or ATP-linked respiration.

45. The method composition or use, of any one of claims 1-44, wherein the subject has a seizure disorder.

46. The method, composition or use of any of the claims 1-45, wherein the subject has a channelopathy, neuronal hyper excitability, lysosomal storage disease (e.g., Pompe and Batten Disease forms (CLN1-13)), Facioscapulohumeral Muscular Dystrophy (FSHD), Dravet Syndrome (SCN1A), NEDAMSS (IRF2BPL), epilepsy and other seizure disorders, seizure disorders caused by SPATA5 mutations, seizures disorders caused by SMARCAL1 mutations, neurological disorders caused by KIF1A mutations, Huntington's disease, SMA with respiratory distress and Charcot-Marie-Tooth Disease 2S (CMT2S), Rett syndrome, Huntington's Disease, Fronto-temporal Dementia, and Multiple Sclerosis, epileptic encephalopathy or a combination thereof.

47. The method, composition or use of any one of the claims 1-46, wherein the subject does not have ALS.

48. A method of identifying a subject who is responsive to CuATSM therapy, comprising analyzing iAstrocytes and/or neurons and/or oligodendrocytes generated from iNPCs derived from a skin cells obtained from the subject for a SCN2A mutation, a mutated SCN2A voltage-gated sodium channel protein, a SLC6A1 mutation, a SCN1A mutation or an IRF2BPL mutation, wherein the subject is identified as a subject who is responsive to CuATSM therapy when the iAstrocytes and/or neurons and/or oligodendrocytes comprise a SCN2A mutation, a mutated SCN2A voltage-gated sodium channel protein, a SLC6A1 mutation, a SCN1A mutation or an IRF2BPL mutation.

49. The method of claim 48, wherein the method further comprises obtaining skin cells from the subject.

50. The method of claim 48 or 49, wherein the method further comprises generating induced neuronal progenitor cells (iNPCs) from skin cells obtained from the subject.

51. The method of any one of claims 48-50, wherein the method further comprises differentiating iNPCs into iAstrocytes and/or neurons and/or oligodendrocytes.

52. The method of any one of claims 48-51, wherein the skin cells obtained from the subject are used to grow primary skin fibroblasts.

53. A method of treating a subject in need thereof, wherein the subject has been identified as a subject who will respond to CuATSM therapy according to the method of any one of claims 48-52, comprising administering CuATSM therapy to the subject.

54. A composition for treating a subject in need thereof, wherein the composition comprises CuATSM and wherein the subject has been identified as a subject who will respond to CuATSM therapy according to the method of any one of claims 48-52.

55. Use of CuATSM for the preparation of a medicament for treating a subject in need thereof, wherein the subject has been identified as a subject who will respond to CuATSM therapy according to the method of any one of claims 48-52.

56. A method of identifying a subject who is responsive to CuATSM therapy, comprising analyzing the level of mitochondrial activity or energy state of astrocytes generated from induced neuronal progenitor cells derived from skin cells obtained from the subject, wherein the subject is identified as a subject who is responsive to CuATSM therapy when the astrocytes exhibit elevated mitochondrial activity compared to astrocytes from a healthy subject.

57. The method of claim 56, wherein the method further comprises a step of obtaining skin cells from the subject.

58. The method of claim 56 or 57, wherein the method further comprises a step of generating induced neuronal progenitor cells (iNPCs) from skin cells obtained from the subject.

59. The method of any one of claims 56-58, wherein the method further comprises differentiating iNPCs into astrocytes or neurons.

60. The method of any one of claims 56-59, wherein the skin cells obtained from the subject are used to grow primary skin fibroblasts.

61. The method of any one of claims 56-60, wherein the mitochondrial activity is analyzed by measuring basal mitochondrial respiration, mitochondrial ATP-linked respiration, or a combination thereof, of the astrocytes.

62. The method of any one of claims 56-61, wherein the energy state is analyzed by measuring oxygen consumption and lactate production or extracellular acidification rate, or a combination thereof of the astrocytes.

63. A method of treating a subject in need thereof, wherein the subject has been identified as a subject who will respond to CuATSM therapy according to the method of any one of claims 56-62, comprising administering CuATSM therapy to the subject.

64. A composition for treating a subject in need thereof, wherein the composition comprises CuATSM and wherein the subject has been identified as a subject who will respond to CuATSM therapy according to the method of any one of claims 56-62.

65. Use of CuATSM for the preparation of a medicament for treating a subject in need thereof, wherein the subject has been identified as a subject who will respond to CuATSM therapy according to the method of any one of claims 56-62.

66. A method of determining effectiveness of CuATSM therapy, comprising analyzing the level of mitochondrial activity of astrocytes generated from induced neuronal progenitor cells derived from skin cells obtained from the subject after administration of CuATSM, wherein an decrease in in basal and/or ATP-linked respiration in the astrocytes, or a decrease in oxidative stress in the astrocytes, or increase in surviving neurons cultured on top of pretreated astrocytes as compared to astrocytes from the subject before administration of CuATSM is indicative of effective CuATSM therapy


SEQUENCE LISTING (4 sequences)

SEQ ID NO: 1 - protein, 2005 aa, Homo Sapiens
MAQSVLVPPGPDSFRFFTRESLAAIEQRIAEEKAKRPKQERKDEDDENGPKPNSDLEAGKSLPFIYGDIPPEMVSVPLEDLDPYYINKKTFIVLNKGKAISRFSATPALYILTPFNPIRKLAIKILVHSLFNMLIMCTILTNCVFMTMSNPPDWTKNVEYTFTGIYTFESLIKILARGFCLEDFTFLRDPWNWLDFTVITFAYVTEFVDLGNVSALRTFRVLRALKTISVIPGLKTIVGALIQSVKKLSDVMILTVFCLSVFALIGLQLFMGNLRNKCLQWPPDNSSFEINITSFFNNSLDGNGTTFNRTVSIFNWDEYIEDKSHFYFLEGQNDALLCGNSSDAGQCPEGYICVKAGRNPNYGYTSFDTFSWAFLSLFRLMTQDFWENLYQLTLRAAGKTYMIFFVLVIFLGSFYLINLILAVVAMAYEEQNQATLEEAEQKEAEFQQMLEQLKKQQEEAQAAAAAASAESRDFSGAGGIGVFSESSSVASKLSSKSEKELKNRRKKKKQKEQSGEEEKNDRVRKSESEDSIRRKGFRFSLEGSRLTYEKRFSSPHQSLLSIRGSLFSPRRNSRASLFSFRGRAKDIGSENDFADDEHSTFEDNDSRRDSLFVPHRHGERRHSNVSQASRASRVLPILPMNGKMHSAVDCNGVVSLVGGPSTLTSAGQLLPEGTTTETEIRKRRSSSYHVSMDLLEDPTSRQRAMSIASILTNTMEELEESRQKCPPCWYKFANMCLIWDCCKPWLKVKHLVNLVVMDPFVDLAITICIVLNTLFMAMEHYPMTEQFSSVLSVGNLVFTGIFTAEMFLKIIAMDPYYYFQEGWNIFDGFIVSLSLMELGLANVEGLSVLRSFRLLRVFKLAKSWPTLNMLIKIIGNSVGALGNLTLVLAIIVFIFAVVGMQLFGKSYKECVCKISNDCELPRWHMHDFFHSFLIVFRVLCGEWIETMWDCMEVAGQTMCLTVFMMVMVIGNLVVLNLFLALLLSSFSSDNLAATDDDNEMNNLQIAVGRMQKGIDFVKRKIREFIQKAFVRKQKALDEIKPLEDLNNKKDSCISNHTTIEIGKDLNYLKDGNGTTSGIGSSVEKYVVDESDYMSFINNPSLTVTVPIAVGESDFENLNTEEFSSESDMEESKEKLNATSSSEGSTVDIGAPAEGEQPEVEPEESLEPEACFTEDCVRKFKCCQISIEEGKGKLWWNLRKTCYKIVEHNWFETFIVFMILLSSGALAFEDIYIEQRKTIKTMLEYADKVFTYIFILEMLLKWVAYGFQVYFTNAWCWLDFLIVDVSLVSLTANALGYSELGAIKSLRTLRALRPLRALSRFEGMRVVVNALLGAIPSIMNVLLVCLIFWLIFSIMGVNLFAGKFYHCINYTTGEMFDVSVVNNYSECKALIESNQTARWKNVKVNFDNVGLGYLSLLQVATFKGWMDIMYAAVDSRNVELQPKYEDNLYMYLYFVIFIIFGSFFTLNLFIGVIIDNFNQQKKKFGGQDIFMTEEQKKYYNAMKKLGSKKPQKPIPRPANKFQGMVFDFVTKQVFDISIMILICLNMVTMMVETDDQSQEMTNILYWINLVFIVLFTGECVLKLISLRYYYFTIGWNIFDFVVVILSIVGMFLAELIEKYFVSPTLFRVIRLARIGRILRLIKGAKGIRTLLFALMMSLPALFNIGLLLFLVMFIYAIFGMSNFAYVKREVGIDDMFNFETFGNSMICLFQITTSAGWDGLLAPILNSGPPDCDPDKDHPGSSVKGDCGNPSVGIFFFVSYIIISFLVVVNMYIAVILENFSVATEESAEPLSEDDFEMFYEVWEKFDPDATQFIEFAKLSDFADALDPPLLIAKPNKVQLIAMDLPMVSGDRIHCLDILFAFTKRVLGESGEMDALRIQMEERFMASNPSKVSYEPITTTLKRKQEEVSAIIIQRAYRRYLLKQKVKKVSSIYKKDKGKECDGTPIKEDTLIDKLNENSTPEKTDMTPSTTSPPSYDSVTKPEKEKFEKDKSEKEDKGKDIRESKK

SEQ ID NO: 2 - DNA, 8776 bp, Homo Sapeins
aacagacattgggtaccatcgaatgactgtcagaacagaaagctaaggcaaaggagggaggatgctgtggtcatcctttcttgtttttttcttctttaatgaggatagagcacatgtgagattttactttctactccagtaaaaattctgaagaattgcattggagactgttatattcaacacatacgtggattctgtgttatgatttacatttttctttatttcagcactttcttatgcaaggagctaaacagtgattaaaggagcaggatgaaaagatggcacagtcagtgctggtaccgccaggacctgacagcttccgcttctttaccagggaatcccttgctgctattgaacaacgcattgcagaagagaaagctaagagacccaaacaggaacgcaaggatgaggatgatgaaaatggcccaaagccaaacagtgacttggaagcaggaaaatctcttccatttatttatggagacattcctccagagatggtgtcagtgcccctggaggatctggacccctactatatcaataagaaaacgtttatagtattgaataaagggaaagcaatctctcgattcagtgccacccctgccctttacattttaactcccttcaaccctattagaaaattagctattaagattttggtacattctttattcaatatgctcattatgtgcacgattcttaccaactgtgtatttatgaccatgagtaaccctccagactggacaaagaatgtggagtatacctttacaggaatttatacttttgaatcacttattaaaatacttgcaaggggcttttgtttagaagatttcacatttttacgggatccatggaattggttggatttcacagtcattacttttgcatatgtgacagagtttgtggacctgggcaatgtctcagcgttgagaacattcagagttctccgagcattgaaaacaatttcagtcattccaggcctgaagaccattgtgggggccctgatccagtcagtgaagaagctttctgatgtcatgatcttgactgtgttctgtctaagcgtgtttgcgctaataggattgcagttgttcatgggcaacctacgaaataaatgtttgcaatggcctccagataattcttcctttgaaataaatatcacttccttctttaacaattcattggatgggaatggtactactttcaataggacagtgagcatatttaactgggatgaatatattgaggataaaagtcacttttattttttagaggggcaaaatgatgctctgctttgtggcaacagctcagatgcaggccagtgtcctgaaggatacatctgtgtgaaggctggtagaaaccccaactatggctacacgagctttgacacctttagttgggcctttttgtccttatttcgtctcatgactcaagacttctgggaaaacctttatcaactgacactacgtgctgctgggaaaacgtacatgatattttttgtgctggtcattttcttgggctcattctatctaataaatttgatcttggctgtggtggccatggcctatgaggaacagaatcaggccacattggaagaggctgaacagaaggaagctgaatttcagcagatgctcgaacagttgaaaaagcaacaagaagaagctcaggcggcagctgcagccgcatctgctgaatcaagagacttcagtggtgctggtgggataggagttttttcagagagttcttcagtagcatctaagttgagctccaaaagtgaaaaagagctgaaaaacagaagaaagaaaaagaaacagaaagaacagtctggagaagaagagaaaaatgacagagtccgaaaatcggaatctgaagacagcataagaagaaaaggtttccgtttttccttggaaggaagtaggctgacatatgaaaagagattttcttctccacaccagtccttactgagcatccgtggctcccttttctctccaagacgcaacagtagggcgagccttttcagcttcagaggtcgagcaaaggacattggctctgagaatgactttgctgatgatgagcacagcacctttgaggacaatgacagccgaagagactctctgttcgtgccgcacagacatggagaacggcgccacagcaatgtcagccaggccagccgtgcctccagggtgctccccatcctgcccatgaatgggaagatgcatagcgctgtggactgcaatggtgtggtctccctggtcgggggcccttctaccctcacatctgctgggcagctcctaccagagggcacaactactgaaacagaaataagaaagagacggtccagttcttatcatgtttccatggatttattggaagatcctacatcaaggcaaagagcaatgagtatagccagtattttgaccaacaccatggaagaacttgaagaatccagacagaaatgcccaccatgctggtataaatttgctaatatgtgtttgatttgggactgttgtaaaccatggttaaaggtgaaacaccttgtcaacctggttgtaatggacccatttgttgacctggccatcaccatctgcattgtcttaaatacactcttcatggctatggagcactatcccatgacggagcagttcagcagtgtactgtctgttggaaacctggtcttcacagggatcttcacagcagaaatgtttctcaagataattgccatggatccatattattactttcaagaaggctggaatatttttgatggttttattgtgagccttagtttaatggaacttggtttggcaaatgtggaaggattgtcagttctccgatcattccggctgctccgagttttcaagttggcaaaatcttggccaactctaaatatgctaattaagatcattggcaattctgtgggggctctaggaaacctcaccttggtattggccatcatcgtcttcatttttgctgtggtcggcatgcagctctttggtaagagctacaaagaatgtgtctgcaagatttccaatgattgtgaactcccacgctggcacatgcatgactttttccactccttcctgatcgtgttccgcgtgctgtgtggagagtggatagagaccatgtgggactgtatggaggtcgctggccaaaccatgtgccttactgtcttcatgatggtcatggtgattggaaatctagtggttctgaacctcttcttggccttgcttttgagttccttcagttctgacaatcttgctgccactgatgatgataacgaaatgaataatctccagattgctgtgggaaggatgcagaaaggaatcgattttgttaaaagaaaaatacgtgaatttattcagaaagcctttgttaggaagcagaaagctttagatgaaattaaaccgcttgaagatctaaataataaaaaagacagctgtatttccaaccataccaccatagaaataggcaaagacctcaattatctcaaagacggaaatggaactactagtggcataggcagcagtgtagaaaaatatgtcgtggatgaaagtgattacatgtcatttataaacaaccctagcctcactgtgacagtaccaattgctgttggagaatctgactttgaaaatttaaatactgaagaattcagcagcgagtcagatatggaggaaagcaaagagaagctaaatgcaactagttcatctgaaggcagcacggttgatattggagctcccgccgagggagaacagcctgaggttgaacctgaggaatcccttgaacctgaagcctgttttacagaagactgtgtacggaagttcaagtgttgtcagataagcatagaagaaggcaaagggaaactctggtggaatttgaggaaaacatgctataagatagtggagcacaattggttcgaaaccttcattgtcttcatgattctgctgagcagtggggctctggcctttgaagatatatacattgagcagcgaaaaaccattaagaccatgttagaatatgctgacaaggttttcacttacatattcattctggaaatgctgctaaagtgggttgcatatggttttcaagtgtattttaccaatgcctggtgctggctagacttcctgattgttgatgtctcactggttagcttaactgcaaatgccttgggttactcagaacttggtgccatcaaatccctcagaacactaagagctctgaggccactgagagctttgtcccggtttgaaggaatgagggttgttgtaaatgctcttttaggagccattccatctatcatgaatgtacttctggtttgtctgatcttttggctaatattcagtatcatgggagtgaatctctttgctggcaagttttaccattgtattaattacaccactggagagatgtttgatgtaagcgtggtcaacaactacagtgagtgcaaagctctcattgagagcaatcaaactgccaggtggaaaaatgtgaaagtaaactttgataacgtaggacttggatatctgtctctacttcaagtagccacgtttaagggatggatggatattatgtatgcagctgttgattcacgaaatgtagaattacaacccaagtatgaagacaacctgtacatgtatctttattttgtcatctttattatttttggttcattctttaccttgaatcttttcattggtgtcatcatagataacttcaaccaacagaaaaagaagtttggaggtcaagacatttttatgacagaagaacagaagaaatactacaatgcaatgaaaaaactgggttcaaagaaaccacaaaaacccatacctcgacctgctaacaaattccaaggaatggtctttgattttgtaaccaaacaagtctttgatatcagcatcatgatcctcatctgccttaacatggtcaccatgatggtggaaaccgatgaccagagtcaagaaatgacaaacattctgtactggattaatctggtgtttattgttctgttcactggagaatgtgtgctgaaactgatctctcttcgttactactatttcactattggatggaatatttttgattttgtggtggtcattctctccattgtaggaatgtttctggctgaactgatagaaaagtattttgtgtcccctaccctgttccgagtgatccgtcttgccaggattggccgaatcctacgtctgatcaaaggagcaaaggggatccgcacgctgctctttgctttgatgatgtcccttcctgcgttgtttaacatcggcctccttcttttcctggtcatgttcatctacgccatctttgggatgtccaattttgcctatgttaagagggaagttgggatcgatgacatgttcaactttgagacctttggcaacagcatgatctgcctgttccaaattacaacctctgctggctgggatggattgctagcacctattcttaatagtggacctccagactgtgaccctgacaaagatcaccctggaagctcagttaaaggagactgtgggaacccatctgttgggattttcttttttgtcagttacatcatcatatccttcctggttgtggtgaacatgtacatcgcggtcatcctggagaacttcagtgttgctactgaagaaagtgcagagcctctgagtgaggatgactttgagatgttctatgaggtttgggagaagtttgatcccgatgcgacccagtttatagagtttgccaaactttctgattttgcagatgccctggatcctcctcttctcatagcaaaacccaacaaagtccagctcattgccatggatctgcccatggtgagtggtgaccggatccactgtcttgacatcttatttgcttttacaaagcgtgttttgggtgagagtggagagatggatgcccttcgaatacagatggaagagcgattcatggcatcaaacccctccaaagtctcttatgagcccattacgaccacgttgaaacgcaaacaagaggaggtgtctgctattattatccagagggcttacagacgctacctcttgaagcaaaaagttaaaaaggtatcaagtatatacaagaaagacaaaggcaaagaatgtgatggaacacccatcaaagaagatactctcattgataaactgaatgagaattcaactccagagaaaaccgatatgacgccttccaccacgtctccaccctcgtatgatagtgtgaccaaaccagaaaaagaaaaatttgaaaaagacaaatcagaaaaggaagacaaagggaaagatatcagggaaagtaaaaagtaaaaagaaaccaagaattttccattttgtgatcaattgtttacagcccgtgatggtgatgtgtttgtgtcaacaggactcccacaggaggtctatgccaaactgactgtttttacaaatgtatacttaaggtcagtgcctataacaagacagagacctctggtcagcaaactggaactcagtaaactggagaaatagtatcgatgggaggtttctattttcacaaccagctgacactgctgaagagcagaggcgtaatggctactcagacgataggaaccaatttaaaggggggagggaagttaaatttttatgtaaattcaacatgtgacacttgataatagtaattgtcaccagtgtttatgttttaactgccacacctgccatatttttacaaaacgtgtgctgtgaatttatcacttttctttttaattcacaggttgtttactattatatgtgactatttttgtaaatgggtttgtgtttggggagagggattaaagggagggaattctacatttctctattgtattgtataactggatatattttaaatggaggcatgctgcaattctcattcacacataaaaaaatcacatcacaaaagggaagagtttacttcttgtttcaggatgtttttagatttttgaggtgcttaaatagctattcgtatttttaaggtgtctcatccagaaaaaatttaatgtgcctgtaaatgttccatagaatcacaagcattaaagagttgttttatttttacataacccattaaatgtacatgtatatatgtatatatgtatatgtgcgtgtatatacatatatatgtatacacacatgcacacacagagatatacacataccattacattgtcattcacagtcccagcagcatgactatcacatttttgataagtgtcctttggcataaaataaaaatatcctatcagtcctttctaagaagcctgaattgaccaaaaaacatccccaccaccactttataaagttgattctgctttatcctgcagtattgtttagccatcttctgctcttggtaaggttgacatagtatatgtcaatttaaaaaataaaagtctgctttgtaaatagtaattttacccagtggtgcatgtttgagcaaacaaaaatgatgatttaagcacactacttattgcatcaaatatgtaccacagtaagtatagtttgcaagctttcaacaggtaatatgatgtaattggttccattatagtttgaagctgtcactgctgcatgtttatcttgcctatgctgctgtatcttattccttccactgttcagaagtctaatatgggaagccatatatcagtggtaaagtgaagcaaattgttctaccaagacctcattcttcatgtcattaagcaataggttgcagcaaacaaggaagagcttcttgctttttattcttccaaccttaattgaacactcaatgatgaaaagcccgactgtacaaacatgttgcaagctgcttaaatctgtttaaaatatatggttagagttttctaagaaaatataaatactgtaaaaagttcattttattttatttttcagccttttgtacgtaaaatgagaaattaaaagtatcttcaggtggatgtcacagtcactattgttagtttctgttcctagcacttttaaattgaagcacttcacaaaataagaagcaaggactaggatgcagtgtaggtttctgcttttttattagtactgtaaacttgcacacatttcaatgtgaaacaaatctcaaactgagttcaatgtttatttgctttcaatagtaatgccttatcattgaaagaggcttaaagaaaaaaaaaatcagctgatactcttggcattgcttgaatccaatgtttccacctagtctttttattcagtaatcatcagtcttttccaatgtttgtttacacagatagatcttattgacccatatggcactagaactgtatcagatataatatgggatcccagctttttttcctctcccacaaaaccaggtagtgaagttatattaccagttacagcaaaatactttgtgtttcacaagcaacaataaatgtagattctttatactgaagctattgacttgtagtgtgttggtgaaatgcatgcaggaaaatgctgttaccataaagaacggtaaaccacattacaatcaagccaaaagaataaaggtttcgcttttgtttttgtatttaattgttgtctttgtttctatctttgaaatgccatttaaaggtagatttctatcatgtaaaaataatctatctgaaaaacaaatgtaaagaacacacattaattactataattcatctttcaattttttcatggaatggaagttaattaagaagagtgtattggataactactttaatattggccaaaaagctagatatggcatcaggtagactagtggaaagttacaaaaattaataaaaaattgactaaca

SEQ ID NO: 3 - protein, 2005 aa, Homo Sapiens
MAQSVLVPPGPDSFRFFTRESLAAIEQRIAEEKAKRPKQERKDEDDENGPKPNSDLEAGKSLPFIYGDIPPEMVSVPLEDLDPYYINKKTFIVLNKGKAISRFSATPALYILTPFNPIRKLAIKILVHSLFNMLIMCTILTNCVFMTMSNPPDWTKNVEYTFTGIYTFESLIKILARGFCLEDFTFLRDPWNWLDFTVITFAYVTEFVNLGNVSALRTFRVLRALKTISVIPGLKTIVGALIQSVKKLSDVMILTVFCLSVFALIGLQLFMGNLRNKCLQWPPDNSSFEINITSFFNNSLDGNGTTFNRTVSIFNWDEYIEDKSHFYFLEGQNDALLCGNSSDAGQCPEGYICVKAGRNPNYGYTSFDTFSWAFLSLFRLMTQDFWENLYQLTLRAAGKTYMIFFVLVIFLGSFYLINLILAVVAMAYEEQNQATLEEAEQKEAEFQQMLEQLKKQQEEAQAAAAAASAESRDFSGAGGIGVFSESSSVASKLSSKSEKELKNRRKKKKQKEQSGEEEKNDRVRKSESEDSIRRKGFRFSLEGSRLTYEKRFSSPHQSLLSIRGSLFSPRRNSRASLFSFRGRAKDIGSENDFADDEHSTFEDNDSRRDSLFVPHRHGERRHSNVSQASRASRVLPILPMNGKMHSAVDCNGVVSLVGGPSTLTSAGQLLPEGTTTETEIRKRRSSSYHVSMDLLEDPTSRQRAMSIASILTNTMEELEESRQKCPPCWYKFANMCLIWDCCKPWLKVKHLVNLVVMDPFVDLAITICIVLNTLFMAMEHYPMTEQFSSVLSVGNLVFTGIFTAEMFLKIIAMDPYYYFQEGWNIFDGFIVSLSLMELGLANVEGLSVLRSFRLLRVFKLAKSWPTLNMLIKIIGNSVGALGNLTLVLAIIVFIFAVVGMQLFGKSYKECVCKISNDCELPRWHMHDFFHSFLIVFRVLCGEWIETMWDCMEVAGQTMCLTVFMMVMVIGNLVVLNLFLALLLSSFSSDNLAATDDDNEMNNLQIAVGRMQKGIDFVKRKIREFIQKAFVRKQKALDEIKPLEDLNNKKDSCISNHTTIEIGKDLNYLKDGNGTTSGIGSSVEKYVVDESDYMSFINNPSLTVTVPIAVGESDFENLNTEEFSSESDMEESKEKLNATSSSEGSTVDIGAPAEGEQPEVEPEESLEPEACFTEDCVRKFKCCQISIEEGKGKLWWNLRKTCYKIVEHNWFETFIVFMILLSSGALAFEDIYIEQRKTIKTMLEYADKVFTYIFILEMLLKWVAYGFQVYFTNAWCWLDFLIVDVSLVSLTANALGYSELGAIKSLRTLRALRPLRALSRFEGMRVVVNALLGAIPSIMNVLLVCLIFWLIFSIMGVNLFAGKFYHCINYTTGEMFDVSVVNNYSECKALIESNQTARWKNVKVNFDNVGLGYLSLLQVATFKGWMDIMYAAVDSRNVELQPKYEDNLYMYLYFVIFIIFGSFFTLNLFIGVIIDNFNQQKKKFGGQDIFMTEEQKKYYNAMKKLGSKKPQKPIPRPANKFQGMVFDFVTKQVFDISIMILICLNMVTMMVETDDQSQEMTNILYWINLVFIVLFTGECVLKLISLRYYYFTIGWNIFDFVVVILSIVGMFLAELIEKYFVSPTLFRVIRLARIGRILRLIKGAKGIRTLLFALMMSLPALFNIGLLLFLVMFIYAIFGMSNFAYVKREVGIDDMFNFETFGNSMICLFQITTSAGWDGLLAPILNSGPPDCDPDKDHPGSSVKGDCGNPSVGIFFFVSYIIISFLVVVNMYIAVILENFSVATEESAEPLSEDDFEMFYEVWEKFDPDATQFIEFAKLSDFADALDPPLLIAKPNKVQLIAMDLPMVSGDRIHCLDILFAFTKRVLGESGEMDALRIQMEERFMASNPSKVSYEPITTTLKRKQEEVSAIIIQRAYRRYLLKQKVKKVSSIYKKDKGKECDGTPIKEDTLIDKLNENSTPEKTDMTPSTTSPPSYDSVTKPEKEKFEKDKSEKEDKGKDIRESKK

SEQ ID NO: 4 - DNA, 8895 bp, Homo Sapiens
aacagacattgggtaccatcgaatgactgtcagaacagaaagctaaggcaaaggagggaggatgctgtggtcatcctttcttgtttttttcttctttaatgaggatagagcacatgtgagattttactttctactccagtaaaaattctgaagaattgcattggagactgttatattcaacacatacgtggattctgtgttatgatttacatttttctttatttcaggggtttttctccctttgcttgacacttctctgtcctgacaccttgagaagaaggatgtgtttgcttacccttccgccatgattgtaaatttcctgaggccttcccagccatgcagcactcactttcttatgcaaggagctaaacagtgattaaaggagcaggatgaaaagatggcacagtcagtgctggtaccgccaggacctgacagcttccgcttctttaccagggaatcccttgctgctattgaacaacgcattgcagaagagaaagctaagagacccaaacaggaacgcaaggatgaggatgatgaaaatggcccaaagccaaacagtgacttggaagcaggaaaatctcttccatttatttatggagacattcctccagagatggtgtcagtgcccctggaggatctggacccctactatatcaataagaaaacgtttatagtattgaataaagggaaagcaatctctcgattcagtgccacccctgccctttacattttaactcccttcaaccctattagaaaattagctattaagattttggtacattctttattcaatatgctcattatgtgcacgattcttaccaactgtgtatttatgaccatgagtaaccctccagactggacaaagaatgtggagtatacctttacaggaatttatacttttgaatcacttattaaaatacttgcaaggggcttttgtttagaagatttcacatttttacgggatccatggaattggttggatttcacagtcattacttttgcgtatgtaacagaatttgtaaacctaggcaatgtttcagctcttcgaactttcagagtcttgagagctttgaaaactatttctgtaattccaggcctgaagaccattgtgggggccctgatccagtcagtgaagaagctttctgatgtcatgatcttgactgtgttctgtctaagcgtgtttgcgctaataggattgcagttgttcatgggcaacctacgaaataaatgtttgcaatggcctccagataattcttcctttgaaataaatatcacttccttctttaacaattcattggatgggaatggtactactttcaataggacagtgagcatatttaactgggatgaatatattgaggataaaagtcacttttattttttagaggggcaaaatgatgctctgctttgtggcaacagctcagatgcaggccagtgtcctgaaggatacatctgtgtgaaggctggtagaaaccccaactatggctacacgagctttgacacctttagttgggcctttttgtccttatttcgtctcatgactcaagacttctgggaaaacctttatcaactgacactacgtgctgctgggaaaacgtacatgatattttttgtgctggtcattttcttgggctcattctatctaataaatttgatcttggctgtggtggccatggcctatgaggaacagaatcaggccacattggaagaggctgaacagaaggaagctgaatttcagcagatgctcgaacagttgaaaaagcaacaagaagaagctcaggcggcagctgcagccgcatctgctgaatcaagagacttcagtggtgctggtgggataggagttttttcagagagttcttcagtagcatctaagttgagctccaaaagtgaaaaagagctgaaaaacagaagaaagaaaaagaaacagaaagaacagtctggagaagaagagaaaaatgacagagtccgaaaatcggaatctgaagacagcataagaagaaaaggtttccgtttttccttggaaggaagtaggctgacatatgaaaagagattttcttctccacaccagtccttactgagcatccgtggctcccttttctctccaagacgcaacagtagggcgagccttttcagcttcagaggtcgagcaaaggacattggctctgagaatgactttgctgatgatgagcacagcacctttgaggacaatgacagccgaagagactctctgttcgtgccgcacagacatggagaacggcgccacagcaatgtcagccaggccagccgtgcctccagggtgctccccatcctgcccatgaatgggaagatgcatagcgctgtggactgcaatggtgtggtctccctggtcgggggcccttctaccctcacatctgctgggcagctcctaccagagggcacaactactgaaacagaaataagaaagagacggtccagttcttatcatgtttccatggatttattggaagatcctacatcaaggcaaagagcaatgagtatagccagtattttgaccaacaccatggaagaacttgaagaatccagacagaaatgcccaccatgctggtataaatttgctaatatgtgtttgatttgggactgttgtaaaccatggttaaaggtgaaacaccttgtcaacctggttgtaatggacccatttgttgacctggccatcaccatctgcattgtcttaaatacactcttcatggctatggagcactatcccatgacggagcagttcagcagtgtactgtctgttggaaacctggtcttcacagggatcttcacagcagaaatgtttctcaagataattgccatggatccatattattactttcaagaaggctggaatatttttgatggttttattgtgagccttagtttaatggaacttggtttggcaaatgtggaaggattgtcagttctccgatcattccggctgctccgagttttcaagttggcaaaatcttggccaactctaaatatgctaattaagatcattggcaattctgtgggggctctaggaaacctcaccttggtattggccatcatcgtcttcatttttgctgtggtcggcatgcagctctttggtaagagctacaaagaatgtgtctgcaagatttccaatgattgtgaactcccacgctggcacatgcatgactttttccactccttcctgatcgtgttccgcgtgctgtgtggagagtggatagagaccatgtgggactgtatggaggtcgctggccaaaccatgtgccttactgtcttcatgatggtcatggtgattggaaatctagtggttctgaacctcttcttggccttgcttttgagttccttcagttctgacaatcttgctgccactgatgatgataacgaaatgaataatctccagattgctgtgggaaggatgcagaaaggaatcgattttgttaaaagaaaaatacgtgaatttattcagaaagcctttgttaggaagcagaaagctttagatgaaattaaaccgcttgaagatctaaataataaaaaagacagctgtatttccaaccataccaccatagaaataggcaaagacctcaattatctcaaagacggaaatggaactactagtggcataggcagcagtgtagaaaaatatgtcgtggatgaaagtgattacatgtcatttataaacaaccctagcctcactgtgacagtaccaattgctgttggagaatctgactttgaaaatttaaatactgaagaattcagcagcgagtcagatatggaggaaagcaaagagaagctaaatgcaactagttcatctgaaggcagcacggttgatattggagctcccgccgagggagaacagcctgaggttgaacctgaggaatcccttgaacctgaagcctgttttacagaagactgtgtacggaagttcaagtgttgtcagataagcatagaagaaggcaaagggaaactctggtggaatttgaggaaaacatgctataagatagtggagcacaattggttcgaaaccttcattgtcttcatgattctgctgagcagtggggctctggcctttgaagatatatacattgagcagcgaaaaaccattaagaccatgttagaatatgctgacaaggttttcacttacatattcattctggaaatgctgctaaagtgggttgcatatggttttcaagtgtattttaccaatgcctggtgctggctagacttcctgattgttgatgtctcactggttagcttaactgcaaatgccttgggttactcagaacttggtgccatcaaatccctcagaacactaagagctctgaggccactgagagctttgtcccggtttgaaggaatgagggttgttgtaaatgctcttttaggagccattccatctatcatgaatgtacttctggtttgtctgatcttttggctaatattcagtatcatgggagtgaatctctttgctggcaagttttaccattgtattaattacaccactggagagatgtttgatgtaagcgtggtcaacaactacagtgagtgcaaagctctcattgagagcaatcaaactgccaggtggaaaaatgtgaaagtaaactttgataacgtaggacttggatatctgtctctacttcaagtagccacgtttaagggatggatggatattatgtatgcagctgttgattcacgaaatgtagaattacaacccaagtatgaagacaacctgtacatgtatctttattttgtcatctttattatttttggttcattctttaccttgaatcttttcattggtgtcatcatagataacttcaaccaacagaaaaagaagtttggaggtcaagacatttttatgacagaagaacagaagaaatactacaatgcaatgaaaaaactgggttcaaagaaaccacaaaaacccatacctcgacctgctaacaaattccaaggaatggtctttgattttgtaaccaaacaagtctttgatatcagcatcatgatcctcatctgccttaacatggtcaccatgatggtggaaaccgatgaccagagtcaagaaatgacaaacattctgtactggattaatctggtgtttattgttctgttcactggagaatgtgtgctgaaactgatctctcttcgttactactatttcactattggatggaatatttttgattttgtggtggtcattctctccattgtaggaatgtttctggctgaactgatagaaaagtattttgtgtcccctaccctgttccgagtgatccgtcttgccaggattggccgaatcctacgtctgatcaaaggagcaaaggggatccgcacgctgctctttgctttgatgatgtcccttcctgcgttgtttaacatcggcctccttcttttcctggtcatgttcatctacgccatctttgggatgtccaattttgcctatgttaagagggaagttgggatcgatgacatgttcaactttgagacctttggcaacagcatgatctgcctgttccaaattacaacctctgctggctgggatggattgctagcacctattcttaatagtggacctccagactgtgaccctgacaaagatcaccctggaagctcagttaaaggagactgtgggaacccatctgttgggattttcttttttgtcagttacatcatcatatccttcctggttgtggtgaacatgtacatcgcggtcatcctggagaacttcagtgttgctactgaagaaagtgcagagcctctgagtgaggatgactttgagatgttctatgaggtttgggagaagtttgatcccgatgcgacccagtttatagagtttgccaaactttctgattttgcagatgccctggatcctcctcttctcatagcaaaacccaacaaagtccagctcattgccatggatctgcccatggtgagtggtgaccggatccactgtcttgacatcttatttgcttttacaaagcgtgttttgggtgagagtggagagatggatgcccttcgaatacagatggaagagcgattcatggcatcaaacccctccaaagtctcttatgagcccattacgaccacgttgaaacgcaaacaagaggaggtgtctgctattattatccagagggcttacagacgctacctcttgaagcaaaaagttaaaaaggtatcaagtatatacaagaaagacaaaggcaaagaatgtgatggaacacccatcaaagaagatactctcattgataaactgaatgagaattcaactccagagaaaaccgatatgacgccttccaccacgtctccaccctcgtatgatagtgtgaccaaaccagaaaaagaaaaatttgaaaaagacaaatcagaaaaggaagacaaagggaaagatatcagggaaagtaaaaagtaaaaagaaaccaagaattttccattttgtgatcaattgtttacagcccgtgatggtgatgtgtttgtgtcaacaggactcccacaggaggtctatgccaaactgactgtttttacaaatgtatacttaaggtcagtgcctataacaagacagagacctctggtcagcaaactggaactcagtaaactggagaaatagtatcgatgggaggtttctattttcacaaccagctgacactgctgaagagcagaggcgtaatggctactcagacgataggaaccaatttaaaggggggagggaagttaaatttttatgtaaattcaacatgtgacacttgataatagtaattgtcaccagtgtttatgttttaactgccacacctgccatatttttacaaaacgtgtgctgtgaatttatcacttttctttttaattcacaggttgtttactattatatgtgactatttttgtaaatgggtttgtgtttggggagagggattaaagggagggaattctacatttctctattgtattgtataactggatatattttaaatggaggcatgctgcaattctcattcacacataaaaaaatcacatcacaaaagggaagagtttacttcttgtttcaggatgtttttagatttttgaggtgcttaaatagctattcgtatttttaaggtgtctcatccagaaaaaatttaatgtgcctgtaaatgttccatagaatcacaagcattaaagagttgttttatttttacataacccattaaatgtacatgtatatatgtatatatgtatatgtgcgtgtatatacatatatatgtatacacacatgcacacacagagatatacacataccattacattgtcattcacagtcccagcagcatgactatcacatttttgataagtgtcctttggcataaaataaaaatatcctatcagtcctttctaagaagcctgaattgaccaaaaaacatccccaccaccactttataaagttgattctgctttatcctgcagtattgtttagccatcttctgctcttggtaaggttgacatagtatatgtcaatttaaaaaataaaagtctgctttgtaaatagtaattttacccagtggtgcatgtttgagcaaacaaaaatgatgatttaagcacactacttattgcatcaaatatgtaccacagtaagtatagtttgcaagctttcaacaggtaatatgatgtaattggttccattatagtttgaagctgtcactgctgcatgtttatcttgcctatgctgctgtatcttattccttccactgttcagaagtctaatatgggaagccatatatcagtggtaaagtgaagcaaattgttctaccaagacctcattcttcatgtcattaagcaataggttgcagcaaacaaggaagagcttcttgctttttattcttccaaccttaattgaacactcaatgatgaaaagcccgactgtacaaacatgttgcaagctgcttaaatctgtttaaaatatatggttagagttttctaagaaaatataaatactgtaaaaagttcattttattttatttttcagccttttgtacgtaaaatgagaaattaaaagtatcttcaggtggatgtcacagtcactattgttagtttctgttcctagcacttttaaattgaagcacttcacaaaataagaagcaaggactaggatgcagtgtaggtttctgcttttttattagtactgtaaacttgcacacatttcaatgtgaaacaaatctcaaactgagttcaatgtttatttgctttcaatagtaatgccttatcattgaaagaggcttaaagaaaaaaaaaatcagctgatactcttggcattgcttgaatccaatgtttccacctagtctttttattcagtaatcatcagtcttttccaatgtttgtttacacagatagatcttattgacccatatggcactagaactgtatcagatataatatgggatcccagctttttttcctctcccacaaaaccaggtagtgaagttatattaccagttacagcaaaatactttgtgtttcacaagcaacaataaatgtagattctttatactgaagctattgacttgtagtgtgttggtgaaatgcatgcaggaaaatgctgttaccataaagaacggtaaaccacattacaatcaagccaaaagaataaaggtttcgcttttgtttttgtatttaattgttgtctttgtttctatctttgaaatgccatttaaaggtagatttctatcatgtaaaaataatctatctgaaaaacaaatgtaaagaacacacattaattactataattcatctttcaattttttcatggaatggaagttaattaagaagagtgtattggataactactttaatattggccaaaaagctagatatggcatcaggtagactagtggaaagttacaaaaattaataaaaaattgactaaca